(19) United States
(12) Patent Application Publication
(10) Pub. No.: US 2026/0308685 A1
(43) Pub. Date: Oct. 8, 2026

(54) HIGHLY SENSITIVE METHOD FOR DETECTING CANCER DNA IN A SAMPLE

(71) Applicant: Inivata Ltd., Cambridge (GB)

(72) Inventors: Malcolm PERRY, Cambridge (GB); Giovanni MARSICO, Cambridge (GB); Robert OSBORNE, Cambridge (GB); Nitzan ROSENFELD, Cambridge (GB); Tim FORSHEW, Hitchin (GB)

(21) Appl. No.: 19/442,820

(22) Filed: Jan. 7, 2026

Related U.S. Application Data
(63) Continuation-in-part of application No. 18/105,215, filed on Feb. 2, 2023.
Application No. 18/105,215 is a continuation-in-part of application No. PCT/IB2021/057217, filed on Aug. 5, 2021.
Application No. 18/105,215 is a continuation-in-part of application No. PCT/IB2022/051195, filed on Feb. 10, 2022.
Application No. PCT/IB2022/051195 is a continuation-in-part of application No. PCT/IB2021/057217, filed on Aug. 5, 2021.
(60) Provisional application No. 63/061,568, filed on Aug. 5, 2020.

(51) Int. Cl. C12Q 1/6886

(52) U.S. Cl. CPC C12Q 1/6886; C12Q 1/6806; C12Q 2600/154; C12Q 2600/156

(57) ABSTRACT

Methods and systems disclosed herein relate to detecting a presence or an absence of a second primary disease in a subject. The methods may comprise performing a workflow comprising a minimal residual disease (MRD) assay on a biological sample and using a result of the workflow to detect a presence or absence of a second primary disease in the subject.

### CROSS-REFERENCE

This application is a continuation-in-part of U.S. application Ser. No. 18/105,215, filed Feb. 2, 2023, which is a continuation-in-part of International Application No. PCT/IB2021/057217, filed on Aug. 5, 2021, which claims the benefit of U.S. Provisional Application No. 63/061,568, filed on Aug. 5, 2020, and a continuation-in-part of International Application No. PCT/IB2022/051195, filed on Feb. 10, 2022, which is a continuation-in-part of International Application No. PCT/IB2021/057217, filed on Aug. 5, 2021, each of which are incorporated by reference herein in their entirety.

### BACKGROUND

The present disclosure relates generally to detection of a primary disease in a subject. More particularly, the present disclosure relates to detection of a second primary disease in a subject that has been previously diagnosed with a first primary disease and performing a minimal residual disease (MRD) assay of a biological sample of the subject.

### SUMMARY

Aspects disclosed herein provide methods for detecting a presence or an absence of a second primary disease in a subject, the method comprising: (a) performing a workflow comprising a first assay and a second assay on a biological sample obtained or derived from the subject, wherein the first assay comprises a minimum residual disease (MRD) assay, wherein the subject has been previously diagnosed with a first primary disease; and (b) using a result of the workflow to detect the presence or the absence of the second primary disease in the subject.

In some embodiments, the first assay comprises a genetic assay, and wherein the second assay comprises an epigenetic assay. In some embodiments, the first assay comprises a tumor-informed genetic assay. In some embodiments, the second assay comprises a tumor-naïve epigenetic assay. In some embodiments, the first assay or the second assay comprises a sequencing assay. In some embodiments, the first assay comprises a personalized sequencing assay, a digital signature assay, or a combination thereof. In some embodiments, the second assay comprises methylation sequencing. In some embodiments, the methylation sequencing comprises enzymatic methyl sequencing (EM-Seq), nanopore sequencing, bisulfite sequencing, or a methylation enrichment assay. In some embodiments, the second assay comprises a fragmentation assay. In some embodiments, the first assay and the second assay are performed in parallel.

In some embodiments, the workflow comprises performing a first analysis using a first signal of the first assay and performing a second analysis using a second signal of the second assay, wherein the first signal and the second signal are derived from different data, and wherein the different data are obtained from the first assay and the second assay. In some embodiments, the first assay comprises a personalized sequencing assay. In some embodiments, the first signal comprises a genetic variant signal. In some embodiments, the second assay comprises a methylation sequencing assay or a fragmentation assay.

In some embodiments, the workflow comprises performing a first analysis using a first signal of the first assay and performing a second analysis using a second signal of the second assay, and wherein the first signal and the second signal originate from different features or different measurements of the same data. In some embodiments, the same data comprises whole genome sequencing data. In some embodiments, the first assay and the second assay each comprises a digital signature assay. In some embodiments, the first signal comprises a genetic variant signal. In some embodiments, the genetic variant signal comprises a single nucleotide variant (SNV) signal. In some embodiments, the second signal comprises an epigenetic change signal. In some embodiments, the epigenetic change signal comprises a DNA methylation signal or a fragmentation signal. In some embodiments, the subject has not been diagnosed with the second primary disease. In some embodiments, the subject is asymptomatic of the second primary disease. In some embodiments, the subject is symptomatic of the second primary disease. In some embodiments, the first primary disease, or the second primary disease, or both, comprises cancer. In some embodiments, the presence of the second primary disease of the subject is detected with a sensitivity of at least 50%. In some embodiments, the absence of the second primary disease of the subject is detected with a specificity of at least 50%. In some embodiments, the biological sample comprises a blood sample, a plasma sample, or a serum sample. In some embodiments, the biological sample comprises cell-free deoxyribonucleic acid (cfDNA), cell-free ribonucleic acid (cfRNA), circulating tumor cells (CTCs), or a combination thereof.

In some embodiments, a time between a suspicion of the second primary disease and a diagnosis of the second primary disease is reduced by at least 10% relative to a method comprising additional testing, additional imaging, or a combination thereof prior to the diagnosis of the second primary disease.

In an aspect, described herein is a method comprising: (a) obtaining a biological sample of a subject; (b) extracting one or more nucleic acids from the biological sample of the subject, wherein the one or more nucleic acids comprise deoxyribonucleic acid (DNA); (c) dividing the extracted one or more nucleic acids into at least two sample aliquots; and (d) sequencing at least a subset of the at least two sample aliquots, using next generation sequencing (NGS) or exome sequencing.

In some embodiments, the biological sample is obtained or derived from a tumor tissue of the subject.

In some embodiments, the biological sample comprises a blood sample.

In some embodiments, the blood sample is whole blood.

In some embodiments, the blood sample comprises a blood plasma sample or a blood serum sample.

In some embodiments, the biological sample comprises a buccal swab sample.

In some embodiments, the biological sample comprises a bone marrow sample.

In some embodiments, the biological sample comprises a plurality of biological samples.

In some embodiments, a first biological sample of the plurality of biological samples comprises a tumor tissue sample of the subject.

In some embodiments, a second biological sample of the plurality of biological samples comprises a normal tissue sample of the subject.

In some embodiments, the method further comprises obtaining the second biological sample from an area adjacent to an area of the first biological sample.

In some embodiments, the DNA is buffy coat DNA.

In some embodiments, the DNA comprises cell-free DNA (cfDNA).

In some embodiments, the method further comprises amplifying the one or more nucleic acids before the sequencing in (d).

In some embodiments, the sequencing comprises generating one or more sequence reads for the at least subset of the at least two sample aliquots.

In some embodiments, the one or more sequence reads comprise a plurality of sequence reads.

In some embodiments, a sequence read of the plurality of sequence reads corresponds to an aliquot of the at least subset of the at least two sample aliquots.

In some embodiments, the subject is a human subject.

In some embodiments, the human subject is a human subject having cancer.

In some embodiments, the at least two sample aliquots comprise about between two and ten sample aliquots.

In some embodiments, the at least two sample aliquots comprise more than ten sample aliquots.

In many cases, cancer treatment may require at least two steps: a first treatment intended to remove the tumor cells then a second treatment aiming to eradicate any remaining cancer cells in the patient's body if the initial treatment is not completely successful. The treatment used to eradicate the remaining cancer cells often differs from the first treatment.

The small number of cancer cells that remain in the person after initial treatment when a patient may apparently be in remission is often called “minimal residual disease” (MRD) or residual disease. These residual cells will ultimately be the cause of relapse in many cancers. It is critical to determine the likelihood of a patient having disease recurrence and relapsing following initial treatment so that those most likely to need additional treatment can receive additional treatment, while those that don't need additional treatment are spared, thereby reducing harm to the patient and decreasing the cost of treatment. As such, effective methods for the detecting minimal residual disease are highly desirable. It is also critical to have sensitive methods that detect risks of cancer recurrence earlier than current methods (e.g., which are usually done by imaging or clinical analysis).

MRD has been successfully detected in some hematological malignancies because relatively large amounts of DNA can be analyzed and the frequency of common tumor specific fusions which can be measured in a straightforward way. There is now strong evidence that MRD can be detected for many solid tumors by assessing cell free DNA (cfDNA) for circulating tumor DNA (ctDNA). The problem with detecting minimal residual disease in cfDNA, however, is that many of the tests used to detect sequence variations in a sample are not sensitive enough. Many of today's molecular tests are done by sequencing cfDNA for a panel of known genes. The problem with detecting minimal residual disease by sequencing cfDNA is that the amount of tumor DNA in cell-free DNA is often well below the limit of detection of such methods. Specifically, the frequency at which an individual tumor sequence variation is expected to occur in the cfDNA of patients that have minimal residual disease is typically well below the frequency at which sequencing artefacts are generated by PCR errors, base mis-calls and/or DNA damage. This problem is compounded by the fact that, in some cases, the level of mutant DNA may be so low that, on average, there is less than a single copy of each mutation being assessed in the cfDNA sample being analyzed. In addition, relatively small amounts of mutant DNA derived from white blood cells that have lysed in the bloodstream can lead to erroneous results. Thus, detection of minimal residual disease by sequencing-based approaches has remained challenging.

This disclosure provides a highly sensitive method for detecting cancer DNA. The method may be used to diagnose minimal residual disease, among other things.

Described below is a method for detecting cancer DNA in a test sample of DNA from a patient. In some embodiments, the method may comprise: (a) sequencing multiple aliquots of the test sample to produce, for each aliquot, sequence reads corresponding to two or more target regions that each have one or more sequence variations present within the patient's cancer; (b) for each aliquot, for each target region: i. determining the number of sequence reads that have the sequence variation; ii. determining the total number of sequence reads; and iii. comparing i. and ii. to one or more error probability distribution models for the sequence variation, wherein the one or more models are obtained from DNA that does not contain the sequence variation; and (c) integrating the collective results of step (b) to determine if there is cancer DNA in the test sample. In any embodiment, step (b) may comprise iv. eliminating variants that are above a threshold in a statistically improbable number of aliquots. These variants (i.e., the variants that are in a statistically improbable number of aliquots) can be identified by measuring the amount of test sample DNA added to each aliquot, calculating the fraction of cancer DNA in the test sample and estimating the probability of observing the number of aliquots with the variant above a threshold based on i and ii

Also described below is a method for detecting cancer DNA in a test sample of DNA from a patient. In some embodiments, the method may comprise: (a) sequencing one or more aliquots of the test sample to produce, for each aliquot, sequence reads corresponding to two or more target regions that each have one or more sequence variations present within the patient's cancer; (b) for each aliquot, for each target region: i. determining the number of sequence reads that have the sequence variation; ii. determining the total number of sequence reads; and iii. comparing i. and ii. to one or more error probability distribution models for the sequence variation, wherein the one or more models are obtained from DNA that does not contain the sequence variation; and (c) integrating the collective results of step (b) to determine if there is cancer DNA in the test sample. In any embodiment, step (b) may comprise iv. eliminating variants that are above a threshold in a statistically improbable number of aliquots. These variants (i.e., the variants that are in a statistically improbable number of aliquots) can be identified by measuring the amount of test sample DNA added to each aliquot, calculating the fraction of cancer DNA in the test sample and estimating the probability of observing the number of aliquots with the variant above a threshold based on i and ii.

Also described below is a method for detecting cancer DNA in a test sample of DNA from a patient. In some embodiments, the method may comprise: (a) sequencing one or more aliquots of the test sample to produce, for each aliquot, sequence reads corresponding to two or more target regions that each have one or more sequence variations present within the patient's cancer; (b) for each aliquot, for each target region: i. determining the number of sequence reads that have the sequence variation; ii. determining the total number of sequence reads; and iii. comparing i. and ii. to one or more error probability distribution models for the sequence variation, wherein the one or more models are obtained from DNA that does not contain the sequence variation; and (c) integrating the collective results of step (b) to determine if there is cancer DNA in the test sample. In any embodiment, step (b) may comprise iv. eliminating variants that are above a threshold in a statistically improbable number of aliquots. These variants (i.e., the variants that are in a statistically improbable number of aliquots) can be identified by measuring the amount of test sample DNA added to each aliquot, calculating the fraction of cancer DNA in the test sample and estimating the probability of observing the number of aliquots with the variant above a threshold based on i and ii.

The present method relies on two features: (i) aliquot-based sequencing (i.e., sequencing the same target regions in multiple aliquots of the same sample, i.e., a sample that has been divided or partitioned) and (ii) analysis of multiple variants assessing for a signal in any of the aliquots (as opposed to identifying variant DNA in one aliquot and then determining that the sample definitely contains cancer DNA because the same variant can be found in another aliquot), and analyzing all of the data, after statistically improbable data points have been removed. The combination of (i) and (ii) allows for a more sensitive test for cancer DNA whilst at the same time reducing the chance of a false positives (both sensitivity and specificity of the test is increased).

One problem solved by this method is that for some samples (i.e., samples that contain a small fraction of cancer DNA, e.g., less than 0.01% tDNA) the number of sequence reads that contain a particular sequence variation is virtually indistinguishable from the variations that are caused by noise (i.e., the combination of base-miscalls, PCR errors, damaged DNA, etc.). As such, in many cases it is simply impossible to reliably determine that a sample contains cancer DNA by conventional sequencing approaches.

As noted above, the present invention is aliquot-based. For example, in some embodiments, the method may involve sequencing at least 10 target regions in at least 3 aliquots of the test sample and, in practice, the method may involve sequencing at least 24 target regions in at least 4 aliquots of the test sample. While aliquot-based sequencing may initially seem like a waste of effort because the same number of wild type and variant molecules are still being sequenced (but split across multiple aliquots, i.e. there is no change in the total amount of DNA being sequenced across the aliquots), the signal-to-noise ratio actually increases in the aliquot-based method. Specifically, in situations in which there are very few variant molecules in the sample (e.g., one or two variant molecules), the ratio of variant molecules to wild type molecules will be much higher in the aliquots that contains the variant molecule (because of the smaller amount of total DNA in each aliquot). This, in turn, eliminates mis-calls and makes the data more reliable. In addition to increasing the signal-to-noise ratio, the method produces more data than conventional approaches, which, in turn, allows the data to be analyzed by more sophisticated statistical and/or threshold-based methods. For example: (i) so called “noisy bases” (i.e., positions that have a high intrinsic background that are frequently miscalled), can be identified and eliminated because the signal will be consistently high (relatively to background) in most or all aliquots and (ii) variants that are associated with improbably high signals (e.g., a variant that has three times the number of sequence reads than would be expected for a single variant molecule in one aliquot and a background number of sequence reads in the other aliquots, or a variant that appears to be in three of four aliquots when the other variants are only in one or zero of the aliquots) can be identified and eliminated. Various other advantages are described below.

Depending on how the method is implemented, the method may have certain advantages over conventional methods. For example, the method may be used to consistently and reliably determine whether a DNA sample has cancer DNA, even if the fraction of cancer DNA in the sample is less than 0.01%. This is well below the level of sensitivity of conventional methods, and well below the frequencies at which sequencing artefacts can be generated by errors. By assessing several sequence variations, the method is also able to detect cancer DNA in a sample of DNA in which there is on average less than a single copy of each individual sequence variation.

The method can be implemented in a way that results in reaching the level of sensitivity without sacrificing specificity (i.e. generating many false positive results). The presence of ctDNA can be estimated at the level of variant molecules added to each aliquot, not variant reads following DNA sequencing. This can reduce false positives in some situations (for example, a low initial input of DNA molecules with high sequencing depth), and provides a more accurate estimate of the global fraction of cancer DNA.

Additionally, in some embodiments, the present method optionally determines whether the sample contains cancer DNA by scoring all variations in all aliquots in a probabilistic continuum (i.e. a probability distribution over the number of molecules observed), rather than calculating the number of positives (the number of aliquots with clear evidence of ctDNA), and determining a positive or negative result through the application of simple rules. This allows exploration of borderline signals which are not significant when taken individually, but can be combined into strong evidence of ctDNA across multiple variants, increasing sensitivity. It also allows for flexible reporting based on degree of confidence, and the potential to combine other data e.g. prior probability of disease recurrence based on cancer type or stage.

In addition, rare errors, such as DNA-damage prior to amplification or early-cycle PCR errors, can be directly modelled by this approach. This would appear to be real signal based on the estimation process described in the previous paragraph. These effects are not captured in most models of DNA sequencing errors and could therefore lead to false positives if left unaccounted for. Alternatively, these can be dealt with by requiring signal detected in aliquots (since 2 such events in a single sample would be very unlikely), however this reduces sensitivity. The method can model this effect by considering whether molecules detected in each aliquot are more likely to come from ctDNA or from a rare error, by considering factors such as the estimated cancer DNA fraction or type of DNA base change.

The method can use a further error-reduction strategy, by excluding variants which show an unusually high level of signal in multiple aliquots, based on the estimated cancer DNA fraction. Intuitively, if only a handful of variant molecules are detected in the sample as a whole, it is unlikely that these would all be present at a single location (barring amplification or copy number changes). This could result from Clonal Hematopoiesis of Indeterminate Potential (CHIP) mutations, contamination, or similar errors. It could also be due to a single DNA base producing many more sequencing errors than accounted for in the background model, which makes this method suitable for “one-shot” use without first sequencing against a panel of non-cancerous samples.

These and other advantages may become apparent in view of the following discussion.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The features of the invention are set forth with particularity in the appended claims. A better understanding of the features and advantages of the present methods and systems will be obtained by reference to the following detailed description that sets forth illustrative examples, in which the principles of the methods and systems are utilized, and the accompanying drawings (also “figure” and “FIG.” herein), of which:

FIG. 1 is a flow chart showing how aliquot-based sequencing can be implemented. As would be apparent, the different aliquots of the test sample can be barcoded with different aliquot identifier sequences and then combined prior to sequencing.

FIG. 2 is a flow chart that follows from the flow chart of FIG. 1. FIG. 2 shows how the sequence reads can be processed to determine, (b) for each aliquot, for each target region, the number of sequence reads that have the sequence variation and the total number of sequence reads.

FIG. 3 is a flow chart that shows an example of how the workflow shown in the flow chart FIG. 2 can be implemented. The steps illustrated in FIG. 3 can be done in any convenient order.

FIG. 4 is a flow chart that follows from the flow chart of FIG. 2. FIG. 4 shows how the variant and total read counts for each sequence variation and aliquot can be analyzed along with probability distributions for each sequence variations and then integrated to determine if there is cancer DNA in the sample.

FIG. 5 is a flow chart illustrating how probability distribution models for each sequence variation can be produced. Probability distributions include binomial, over-dispersed binomial, beta, normal, exponential or gamma probability distribution models. Such models may not be needed in embodiments that use molecular indexes.

FIG. 6 is a flow chart illustrating a threshold-based approach for analyzing data for each sequence variation in each aliquot.

FIG. 7 is a flow chart that illustrates a way to integrate the results of the threshold-based method illustrated in FIG. 6.

FIG. 8 is a flow chart illustrating a statistical approach for analyzing data for each sequence variation in each aliquot.

FIG. 9 is a flow chart illustrating how the statistical results shown in FIG. 8 can be integrated.

FIG. 10 is a flow chart illustrating the last step in FIG. 1, showing two approaches by which the results of one test sample can be compared to one or more additional samples.

FIG. 11 schematically illustrates some of the principles of an embodiment of the present method.

FIG. 12 illustrates the principles of a probability distribution for estimating the number of variant molecules.

FIGS. 13A and 13B illustrate examples of error probability distributions. In the model shown in FIG. 13A, the data corresponding to low frequency high signal events are hatched. The model shown in FIG. 13B is a mixture model. “VAF” refers to variant allele fraction. Such models are obtained from DNA that does not contain the sequence variation and they indicate the probability of different variant allele fractions in this non-cancerous DNA (or the no of variant reads over the total wt reads). Such distributions may differ from variant class to variant class and sequence depth to sequencing depth. In some cases, 2 or more distributions are required to account for the different types of error. In some cases, a threshold may be established in which one can be reasonably certain that a sequence variation identified in sequence reads is not an error.

FIG. 14 illustrates how data from “noisy” bases can be identified and eliminated using an aliquot approach.

FIG. 15 illustrates some of the difficulties in detecting cancer DNA by methods in which the individual aliquots are scored for whether they contain a particular variant or not.

FIG. 16 shows how the fraction of cancer DNA can be calculated.

FIGS. 17A and 17B show the results of an experiment in which over 40 sequence variations in four aliquots of each of three different samples containing varying levels of circulating tumor (ctDNA) were assessed.

FIG. 18 are histograms showing the observed frequency of certain substitution classes in control samples.

FIGS. 19A-19C are histograms showing how one or more beta-binomial error probability distributions can describe substitution classes having one or more maxima.

### DETAILED DESCRIPTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As may be apparent, each assay assessing multiple aliquots for two or more target regions may have a different lower limit at which it can reliably detect cancer DNA, sometimes referred to as Limit of Detection or LOD. The LOD may be calculated empirically, for example, using standard cell line dilutions, or it may be calculated on a patient-by-patient basis. It may also have a different limit at which amounts of cancer DNA can be accurately quantified, sometimes referred to as Limit of Quantification or LOQ. For such an assay to be most useful, in some cases it may be valuable to obtain an accurate estimate of either or both of the LOD or LOQ. Such an estimate can be obtained by combining factors which may include clonality, mappability, estimated error rate, estimated rate of high signal background events, presence within a region of copy number gain or amplification for each sequence variation associated with the patient's cancer that is targeted, and the number of target regions. It may also include library preparation and sequencing run specific factors which may include: the number of aliquots, the total number of sequencing reads for the targeted regions and the number of molecules input into each aliquot.

### Definitions

Unless otherwise defined, all technical and scientific terms used herein have the same meaning as commonly understood by one of ordinary skill in the art to which this invention belongs. Still, certain elements are defined for the sake of clarity and ease of reference.

Terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); (the contents of which are incorporated by reference in their entireties) and the like.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, greater than 10,000 bases, greater than 100,000 bases, greater than about 1,000,000, up to about 1010 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylenecarbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid,” or “UNA,” is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is incorporated by reference herein for disclosure of UNA.

The term “nucleic acid sample,” as used herein, denotes a sample containing nucleic acids. Nucleic acid samples used herein may be complex in that they contain multiple different molecules that contain sequences. Genomic DNA samples from a mammal (e.g., mouse or human) are types of complex samples. Complex samples may have more than about 104, 105, 106 or 107, 108, 109 or 1010 different nucleic acid molecules. Any sample containing nucleic acid, e.g., genomic DNA from tissue culture cells or a sample of tissue, may be employed herein.

The term “oligonucleotide” as used herein denotes a single-stranded multimer of nucleotide of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) or deoxyribonucleotide monomers, or both ribonucleotide monomers and deoxyribonucleotide monomers. An oligonucleotide may be 10 to 20, 21 to 30, 31 to 40, 41 to 50, 51 to 60, 61 to 70, 71 to 80, 80 to 100, 100 to 150 or 150 to 200 nucleotides in length, for example.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers are extended by a DNA polymerase. Primers are generally of a length compatible with their use in synthesis of primer extension products, and are usually in the range of 8 to 200 nucleotides in length, such as 10 to 100 or 15 to 80 nucleotides in length. A primer may contain a 5′ tail that does not hybridize to the template.

Primers are usually single-stranded for maximum efficiency in amplification, but may alternatively be double-stranded or partially double-stranded. Also included in this definition are toehold exchange primers, as described in Zhang et al (Nature Chemistry 2012 4:208-214), which is incorporated by reference herein.

Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

The term “hybridization” or “hybridizes” refers to a process in which a region of nucleic acid strand anneals to and forms a stable duplex, either a homoduplex or a heteroduplex, under normal hybridization conditions with a second complementary nucleic acid strand, and does not form a stable duplex with unrelated nucleic acid molecules under the same normal hybridization conditions. The formation of a duplex is accomplished by annealing two complementary nucleic acid strand regions in a hybridization reaction. The hybridization reaction can be made to be highly specific by adjustment of the hybridization conditions under which the hybridization reaction takes place, such that two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press, the contents of which are hereby incorporated by reference in their entireties. As used herein, the term “hybridizing” or “hybridization” refers to any process by which a strand of nucleic acid binds with a complementary strand through base pairing.

A nucleic acid is considered to be “selectively hybridizable” to a reference nucleic acid sequence if the two sequences specifically hybridize to one another under moderate to high stringency hybridization conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y., the contents of which are hereby incorporated by reference in its entirety).

The term “duplex,” or “duplexed,” as used herein, describes two complementary polynucleotide region that are base-paired, i.e., hybridized together.

“Genetic locus,” “locus,”, “locus of interest”, “region” or “segment” in reference to a genome or target polynucleotide, means a contiguous sub-region or segment of the genome or target polynucleotide. As used herein, genetic locus, locus, or locus of interest may refer to the position of a nucleotide, a gene or a portion of a gene in a genome or it may refer to any contiguous portion of genomic sequence whether or not it is within, or associated with, a gene, e.g., a coding sequence. A genetic locus, locus, or locus of interest can be from a single nucleotide to a segment of a few hundred or a few thousand nucleotides in length or more. In general, a locus of interest will have a reference sequence associated with it (see description of “reference sequence” below).

The terms “plurality”, “population” and “collection” are used interchangeably to refer to something that contains at least 2 members. In certain cases, a plurality, population or collection may have at least 5, at least 10, at least 100, at least 1,000, at least 10,000, at least 100,000, at least 106, at least 107, at least 108 or at least 109 or more members.

The term “sample identifier sequence”, “sample index”, “multiplex identifier” or “MID” is a sequence of nucleotides that is appended to a target polynucleotide, where the sequence identifies the source of the target polynucleotide (i.e., the sample from which sample the target polynucleotide is derived). In use, each sample is tagged with a different sample identifier sequence (e.g., one sequence is appended to each sample, where the different samples are appended to different sequences), and the tagged samples are pooled. After the pooled sample is sequenced, the sample identifier sequence can be used to identify the source of the sequences. A sample identifier sequence may be added to the 5′ end of a polynucleotide or the 3′ end of a polynucleotide. In certain cases, some of the sample identifier sequence may be at the 5′ end of a polynucleotide and the remainder of the sample identifier sequence may be at the 3′ end of the polynucleotide. When elements of the sample identifier have sequence at each end, together, the 3′ and 5′ sample identifier sequences identify the sample. In many examples, the sample identifier sequence is only a subset of the bases which are appended to a target oligonucleotide. An identifier sequence can be appended to a polynucleotide by ligation or by primer extension. In the latter embodiments, the identifier sequence may be in the 5′ tail or the primer used for primer extension. In such embodiments the target polynucleotide is a copy of the original target polynucleotide.

The term “aliquot identifier sequence” refers to an appended sequence that allows sequence reads from different aliquots to be distinguished from one another. Aliquot identifier sequences work in the same way as sample identifier sequences described above, except that they are used on aliquots of a sample, rather than different samples. A single sequence may serve as a sample identifier and an aliquot identifier.

The term “variable”, in the context of two or more nucleic acid sequences that are variable, refers to two or more nucleic acids that have different sequences of nucleotides relative to one another. In other words, if the polynucleotides of a population have a variable sequence, then the nucleotide sequence of the polynucleotide molecules of the population may vary from molecule to molecule. The term “variable” is not to be read to require that every molecule in a population has a different sequence to the other molecules in a population.

The term “substantially” refers to sequences that are near-duplicates as measured by a similarity function, including but not limited to a Hamming distance, Levenshtein distance, Jaccard distance, cosine distance etc. (see, generally, Kemena et al, Bioinformatics 2009 25:2455-65, the contents of which are hereby incorporated by reference in its entirety). The exact threshold depends on the error rate of the sample preparation and sequencing used to perform the analysis, with higher error rates requiring lower thresholds of similarity. In certain cases, substantially identical sequences have at least 98% or at least 99% sequence identity.

The term “sequence variation”, as used herein, is a variant that is different to a reference sequence, such as a reference genome or sequence from a sample of a patient not anticipated to contain somatic variants such as a buccal swab. In many instances a “sequence variation” is a variant that is present at a frequency of less than 50%, relative to other molecules in the sample. Many sequence variations, e.g., indels and nucleotide substitutions, are substantially identical to the molecules that do not contain the sequence variation. In some cases, a particular sequence variation may be present in a sample at a frequency of less than 20%, less than 10%, less than 5%, less than 1%, less than 0.5%, less than 0.1%, less than 0.05% or less than 0.01%.

The term “reference sequence”, as used herein, is a reference sequence from a reference genome or sequence from a sample of a patient not anticipated to contain somatic variants such as a buccal swab. A reference sequence corresponds to a sequence (e.g. a target sequence) that contains or may be suspected of containing a “sequence variation”, hence enabling the existence (or not) of a sequence variation to be determined by comparing the sequence (e.g. the target sequence) that contains or may be suspected of containing a sequence variation to the reference sequence. A reference sequence differs from the sequence (e.g. a target sequence) that contains or may be suspected of containing the sequence variation only in the sequence variation itself, since the reference sequence and the sequence (e.g. a target sequence) that contains or may be suspected of containing a sequence variation originates from the same genomic location.

The term “reference genome”, as used herein, may refer to a single genome, a collection of genomes, or a consensus genome. The reference genome may be from one or more publicly available databases. Reference genomes are used to determine the location of a sequence that is being analysed in the organism's genome. As the skilled person would be aware, a consensus genome is a genome that is constructed from multiple genomes from the same species.

The term “nucleic acid template” is intended to refer to the initial nucleic acid molecule that is copied during amplification. Copying in this context can include the formation of the complement of a particular single-stranded nucleic acid. The “initial” nucleic acid can comprise nucleic acids that have already been processed, e.g., amplified, extended, labeled with adaptors, etc.

The term “tailed”, in the context of a tailed primer or a primer that has a 5′ tail, refers to a primer that has a region (e.g., a region of at least 12-50 nucleotides) at its 5′ end that does not hybridize or partially hybridizes to the same target as the 3′ end of the primer.

The term “initial template” refers to a sample that contains a target sequence to be amplified. The term “amplifying” as used herein refers to generating one or more copies of a target nucleic acid, using the target nucleic acid as a template.

The term “amplicon” as used herein refers to the product (or “band”) amplified by a particular pair of primers in a PCR reaction.

The “replicate amplicon” as used herein refers to the same amplicon amplified using different portions or aliquots of a sample. Replicate amplicons typical have near identical sequences, except for sequence variations in the template, PCR errors, and differences in the sequences of the primers used for each aliquot (e.g., differences in the 5′ ends of the primers such as in the aliquot identifier sequence, etc.).

A “polymerase chain reaction” or “PCR” is an enzymatic reaction in which a specific template DNA is amplified using one or more pairs of sequence specific primers.

“PCR conditions” are the conditions in which PCR is performed, and include the presence of reagents (e.g., nucleotides, buffer, polymerase, etc.) as well as temperature cycling (e.g., through cycles of temperatures suitable for denaturation, renaturation and extension), as is known in the art.

A “multiplex polymerase chain reaction” or “multiplex PCR” is an enzymatic reaction that employs two or more primer pairs for different targets templates. If the target templates are present in the reaction, a multiplex polymerase chain reaction results in two or more amplified DNA products that are co-amplified in a single reaction using a corresponding number of sequence-specific primer pairs.

The term “next generation sequencing” refers to the so-called highly parallelized methods of performing nucleic acid sequencing and comprises the sequencing-by-synthesis or sequencing-by-ligation platforms currently employed by Illumina®, Life Technologies, Pacific Biosciences and Roche, etc. Next generation sequencing methods may also include, but not be limited to, nanopore sequencing methods such as offered by Oxford Nanopore or electronic detection-based methods such as the Ion Torrent technology commercialized by Life Technologies.

The term “sequence read” refers to the output of a sequencer. A sequence read typically contains a string of Gs, As, Ts and Cs, of 50-1000 or more bases in length and, in many cases, each base of a sequence read may be associated with a score indicating the quality of the base call.

The terms “assessing the presence of” and “evaluating the presence of” include any form of measurement, including determining if an element is present and estimating the amount of the element. The terms “determining”, “measuring”, “evaluating”, “assessing” and “assaying” are used interchangeably and include quantitative and qualitative determinations. Assessing may be relative or absolute. “Assessing the presence of” includes determining the amount of something present, and/or determining whether it is present or absent.

If two nucleic acids are “complementary,” they hybridize with one another under high stringency conditions. The term “perfectly complementary” is used to describe a duplex in which each base of one of the nucleic acids base pairs with a complementary nucleotide in the other nucleic acid. In many cases, two sequences that are complementary have at least 10, e.g., at least 12 or 15 nucleotides of complementarity.

An “oligonucleotide binding site” refers to a site to which an oligonucleotide hybridizes in a target polynucleotide. If an oligonucleotide “provides” a binding site for a primer, then the primer may hybridize to that oligonucleotide or its complement.

The term “strand” as used herein refers to a nucleic acid made up of nucleotides covalently linked together by covalent bonds, e.g., phosphodiester bonds. In a cell, DNA usually exists in a double-stranded form, and as such, has two complementary strands of nucleic acid referred to herein as the “top” and “bottom” strands. In certain cases, complementary strands of a chromosomal region may be referred to as “plus” and “minus” strands, the “first” and “second” strands, the “coding” and “noncoding” strands, the “Watson” and “Crick” strands or the “sense” and “antisense” strands. The assignment of a strand as being a top or bottom strand is arbitrary and does not imply any particular orientation, function or structure. The nucleotide sequences of the first strand of several exemplary mammalian chromosomal regions (e.g., BACs, assemblies, chromosomes, etc.) is known, and may be found in NCBI's Genbank database, for example.

The term “extending”, as used herein, refers to the extension of a primer by the addition of nucleotides using a polymerase. If a primer that is annealed to a nucleic acid is extended, the nucleic acid acts as a template for extension reaction.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide is obtained.

The term “pooling”, as used herein, refers to the combining, e.g., mixing, of two or more samples or aliquots of a sample such that the molecules within those samples or aliquots become interspersed with one another in solution.

The term “pooled sample”, as used herein, refers to the product of pooling.

The term “portion”, as used herein in the context of different portions of the same sample, refers to an aliquot or part of a sample. For example, if one microliter of 100 μl sample is added to each of 10 different PCR reactions, then those reactions each contain different portions of the same sample.

As used herein, the term “cell-free DNA” (“cfDNA”) refers to DNA that is free in a bodily fluid, not contained in cells. cfDNA can be isolated from, for example, whole blood, blood plasma, blood serum, cerebrospinal fluid, urine, saliva, stool, amniotic fluid, aqueous humour, bile, breast milk, cerumen, chyle, exudates, gastric juice, lymph, mucus, pericardial fluid, peritoneal fluid, pleural fluid, pus, sebum, serous fluid, semen, sputum, synovial fluid, sweat, tears, or vomit for example. “Cell-free DNA from the bloodstream” and “circulating cell-free DNA” refers to DNA that is circulating in the peripheral blood of a patient. The DNA molecules in cell-free DNA may have a median size that is below 1 kb (e.g., in the range of 50 bp to 500 bp, 80 bp to 400 bp, or 100-1,000 bp), although fragments having a median size outside of this range may be present. Cell-free DNA may contain tumor DNA (tDNA), e.g., tumor DNA circulating freely in the blood of a cancer patient. cfDNA can be obtained by centrifuging the sample to remove all cells, and then isolating the DNA from the remaining liquid (e.g., plasma or serum). Such methods are well known (see, e.g., Lo et al, Am J Hum Genet 1998; 62:768-75). Circulating cell-free DNA can be double-stranded or single-stranded. This term is intended to encompass free DNA molecules that are circulating in the bloodstream as well as DNA molecules that are present in extra-cellular vesicles (such as exosomes) that are circulating in the bloodstream. As used herein, the term “bodily fluid” includes any fluid produced by the living body. For example, bodily fluid includes, but is not limited to, amniotic fluid, aqueous humour, bile, blood plasma, blood serum, breast milk, cerebrospinal fluid, cerumen, chyle, exudates, gastric juice, lymph, mucus, pericardial fluid, peritoneal fluid, pleural fluid, pus, saliva, sebum, serous fluid, semen, stool, sputum, synovial fluid, sweat, tears, urine, vomit and whole blood.

As used herein, the term “tumor DNA” (or “tDNA”) is tumor-derived DNA. tDNA can be identified because it contains mutations. tDNA can be isolated directly from a tissue biopsy, from circulating tumor cells (CTCs), from other cells that are no longer part of the tumor tissue but are not circulating such as those in the urine or stool samples, or it may be part of (a “fraction of”) the cfDNA of a patient (in which case it may be referred to as circulating tumour DNA, ctDNA). tDNA includes both clonal and sub-clonal mutations. In the evolution of a tumor, there is a transition between clonal and sub-clonal mutations. Sub-clonal mutations are only present in a subset of cells in the tumor: these occur after the most recent common ancestor of all cancer cells in the tumor sample. In contrast, clonal mutations occurred before the most recent common ancestor of all cancer cells. Clonal mutations are therefore present in all cells in the tumor unless there is some mechanism that has removed the mutation e.g. a structural variation in which case the entire locus will be lost in a subset of cells. ctDNA is of tumor origin and originates directly from the tumor or from circulating tumor cells (CTCs), which are viable, intact tumor cells that shed from primary tumors and can enter the bloodstream or lymphatic system. The precise mechanism of how ctDNA is released is unclear, although it is postulated to involve apoptosis and necrosis from dying cells, or active release from viable tumor cells. Circulating tDNA (ctDNA) can be highly fragmented and in some cases can have a mean fragment size about 100-250 bp, e.g., 150 to 200 bp long. The amount of ctDNA in a sample of circulating cell-free DNA isolated from a cancer patient varies greatly: typical samples contain less than 10% ctDNA, although many samples from patients being assessed for MRD may have less than 0.01% ctDNA and some samples have over 10% ctDNA. Molecules of ctDNA can be often identified because they contain tumorigenic mutations.

As used herein, the term “sequence variation” refers to the combination of a position and type of a sequence alteration. For example, a sequence variation can be referred to by the position of the variation and which type of substitution (e.g., G to A, G to T, G to C, A to G, etc. or insertion/deletion of a G, A, T or C, etc.) is present at the position. A sequence variation may be a substitution, deletion, insertion rearrangement of one or more nucleotides. In the context of the present method, a sequence variation can be generated by, e.g., a PCR error, an error in sequencing or a genetic variation.

As used herein, the term “genetic variation” refers to a variation (e.g., a nucleotide substitution, an indel or a rearrangement) that is present or deemed as being likely to be present in a nucleic acid sample. A genetic variation can be from any source. For example, a genetic variation can be generated by a mutation (e.g., a somatic mutation), or it can be germ line such as in an organ transplant or pregnancy. If sequence variation is called as a genetic variation, the call indicates that the sample likely contains the variation; in some cases a “call” can be incorrect. In many cases, the term “genetic variation” can be replaced by the term “mutation”. For example, if the method is being used to detect sequence variations that are associated with cancer or other diseases that are caused by mutations, then “genetic variation” can be replaced by the term “mutation”.

As used herein, depending on the context the term “calling” can mean indicating whether a particular genetic variation is present in a sequence, whether a sample contains a genetic variation or whether sample contains cancer DNA.

As used herein, the term “threshold” refers to a level of evidence (e.g., a ratio) that is required to make a call.

As used herein, the term “value” refers to a number, letter, word (e.g., “high”, “medium” or “low”) or descriptor (e.g., “+++” or “++”) that can indicate the strength of evidence. A value can contain one component (e.g., a single number) or more than one component, depending on how a value is analyzed.

As used herein, the term “Limit of Detection” or “LOD” refers to the lower limit at which each assay can reliably detect cancer DNA at a stated probability. The probability may be 99%, 95%, 90% or any other stated probability. The LOD may be calculated empirically using standard cell line dilutions, or it may be calculated on a patient-by-patient basis.

As used herein, the term “Limit of Quantification” or “LOQ” of an assay refers to the lower limit at which amounts of cancer DNA can be accurately quantified. The LOQ could be the same as the LOD, or it may be higher.

The LOD and the LOQ may be used separately for each assay, or they may be used together. For example, in some cases it may be valuable to obtain an accurate estimate of either or both of the LOD or LOQ. Such an estimate can be obtained by combining factors which may include clonality, mappability, estimated error rate, estimated rate of high signal background events, presence within a region of copy number gain or amplification for each sequence variation associated with the patient's cancer that is targeted. It may also include library preparation and sequencing run specific factors which may include: the number of aliquots, the total number of sequencing reads for the targeted regions, the number of molecules input into each aliquot, and the total number of targeted regions. Generally, increasing the number of targeted regions will improve the LOD or LOQ.

As used herein, the term “aliquot” refers to a portion of a sample. For example, if three volumes are independently removed from the same sample, each of the volumes can be referred to as an aliquot. Aliquots do not need to be the same volume.

As used herein, the term “cancer-associated cells” means cells that are part of or genetically related to the cells of a patient's cancer. Cancer-associated cells can be part of a solid tumor a blood/haematological cancer or a solid tumor. The presence of cancer-associated cells in a patient may be a sign that all cancer cells were not removed or killed during treatment. The cancer-associated cells have substantially the same somatic mutations as the cells of the patient's cancer and, in some cases, may be progeny of one or more cells of a cancer. Cancer-associated cells may result from minimal residual disease or they could be generated by incomplete removal of a tumor, incomplete treatment, cancer recurrence or relapse at a primary or distal site and/or tumor metastasis (including micrometastasis).

As used herein, the term “sequence variation associated with (or present within) the patient's cancer” is intended to mean a somatic mutation that is in the genome of cells of the patient's cancer or was in the genome of cells of the patient's cancer prior to any cancer treatment. It can also mean epigenetic changes present within a cancer sample.

As used herein, the term “minimal residual disease” (MRD), refers to the presence of cancer cells following a treatment with curative intent. MRD may also be referred to as “molecular residual disease” or residual disease” in some publications.

As used herein, the term “detecting recurrence” refers to detecting the recurrence of a tumor through the identification of mutant DNA. In this context, the term “early detection” refers to the detection of mutant DNA before tumor recurrence can be reliably detected through conventional standard-of-care/surveillance monitoring methods such as radiological imaging etc. This may be achieved for example by monitoring serially collected blood samples at a plurality of time points for the presence of ctDNA in cfDNA, as described below.

The term “cancer” is used herein to refer to any disease characterized by uncontrolled cell division. A cancer can be a cancer of the blood (i.e., haematological cancer), e.g., leukemia, lymphoma, or multiple myeloma, or a cancer can be neoplastic, e.g., associated with an abnormal mass of tissue in which cells grow and divide more than they should or do not die when they should. Neoplastic cancers, e.g., lung, breast or liver cancer, are associated with a solid tumor.

The term “cancer DNA” refers to DNA that is from cancerous cells. Cancer DNA may be present in DNA isolated from a population of cells that are isolated from lymph, bone marrow or the circulating blood of a patient, if the patient has a blood cancer. Cancer DNA from a solid tumor can be found in cfDNA, in which case it is referred to tDNA or ctDNA.

The term “probability” refers to the chance of a particular outcome occurring, or how likely that outcome is to occur. Probability may be based on the values of parameters in a model. Probability refers to unknown events, and attaches to possible results. Since possible results are mutually exclusive and exhaustive, a probability can be expressed on a linear scale. For example, a probability may be expressed as a value between 0 (impossible) and 1 (certain), or may equally be expressed as a percentage or fraction. For example, in the context of the present invention, a probability may be used as a measure to determine whether cancer DNA is present in a sample.

The term “likelihood” refers to the hypothetical probability of a specific outcome being yielded by an event that has already occurred. Likelihood is used to assess how well a sample provides support for particular values of a parameter in a model. Likelihood therefore refers to past events with known outcomes, and attaches to hypotheses. Since different hypotheses are neither mutually exclusive nor exhaustive, likelihoods attached to hypotheses have meaning as a relative likelihood, e.g. a ratio of two likelihoods (Bayes factor).

The term “likelihood ratio” (LRi) refers to a ratio of at least two likelihoods, each attached to a different hypothesis, which can be used to determine which hypothesis is more likely given an experimental result. Likelihood ratios can be used as a measure of diagnostic accuracy since they can be used to determine the potential utility of a particular diagnostic test, and how likely it is that a patient has a disease or condition. The LRi of any clinical finding is the probability of that finding in patients with disease, divided by the probability of the same finding in patients without disease. For example, a likelihood ratio may be calculated between the likelihood of observing the estimates in (b) in samples: (i) if cancer DNA is present (ii) if cancer DNA is not present. Individual likelihood ratios LRi may be combined into a cumulative LR score (product of LRi equivalent to sum of log-likelihoods) across all regions and aliquots of a sample. For example, in the context of the present invention, a likelihood ratio may be used as a measure to determine whether cancer DNA is present in a sample.

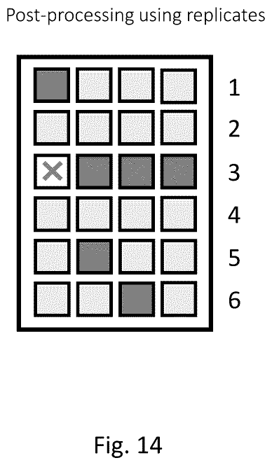

The terms “error probability distribution” and “error probability distribution model” refer to a distribution that estimates or models the probability that an observation (typically a variant allele fraction) is due to error. These terms capture both “high signal background events” (which may be due to DNA damage or very early cycle PCR errors) and “estimated background error rate” (which includes sequencer and PCR polymerase “errors”). Examples of such distributions are shown in FIGS. 13A and B.

The term “collective” in the context of analyzing “collective results” means the results for all of the variants and aliquots (excluding any statistical outliers or other variants excluded for example as they are not present in the cancer DNA or are present in buffy coat DNA), not just a positive result.

The term “target region” refers to a region of DNA that contains or is suspected of containing one or more sequence variations, but excluding “control regions”. The methods of the invention are designed to sequence one or more target regions for each aliquot.

The term “control region” refers to a region of DNA that does not contain or is not expected to contain a somatic sequence variation. Methods of the invention may sequence one or more control regions as a control to, for example, ensure the sequencing reaction has taken place correctly, check for contamination, check for sample mix-ups and/or sampling labeling errors. For example, control regions may be used to estimate an error rate for a test sample; if the error rate is higher than expected (perhaps due to a poor sequencing reaction and/or reagents), a higher threshold may be used for calling target regions. A collection of control regions can be used as a genomic identifier or fingerprint for different patients, since the sequences of the control regions should be the same between different assays analyzing samples from the same patient. Control regions generally contain one or more germline polymorphism(s) to allow this patient-specific genomic profile to be generated. In some embodiments, control regions may include copy number polymorphisms and/or small polymorphic insertions and deletions. Control regions generally are sequenced in the same sequencing reaction as target regions. Accordingly, in any embodiment, the method can comprise sequencing one or more aliquots of a test sample to produce, for each aliquot, sequence reads corresponding to two or more target regions that each have one or more sequence variations present within the patient's cancer and at least one control region.

Other definitions of terms may appear throughout the specification. It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

As noted above, a method for detecting cancer DNA in a test sample of DNA from a patient (e.g., a cancer patient) is provided. In some embodiments, the method may comprise measuring a level or intensity of signal in one or more aliquots. In some embodiments, measuring a level of signal in one or more aliquots may comprise sequencing one or more aliquots. In some embodiments, the method may comprise sequencing multiple aliquots of the test sample (e.g., at least 2, at least 3, at least 4, at least 5 or at least 6 aliquots of the sample) to produce, for each aliquot, sequence reads corresponding to two or more target regions (e.g., at least three, at least 5, at least 10, at least 20, at least 50, at least 100, at least 1000 or at least 5000 target regions) that each have one or more sequence variations present within the patient's cancer. For example, the method may involve sequencing 3-10 aliquots of the test DNA sample to produce, for each aliquot, sequence reads corresponding to 6-100 target regions. In very general terms, sensitivity can be increased by increasing the number of aliquots, by increasing the number of variants, or by increasing the number of aliquots and variants. For example, in some embodiments the method may comprise sequencing at least two (e.g., three or four) aliquots of the test sample to produce, for each aliquot, sequence reads corresponding to ten or more target regions that each have one or more sequence variations. In other embodiments the method may comprise sequencing at least ten aliquots of the test sample to produce, for each aliquot, sequence reads corresponding to two (e.g., three or four) or more target regions that each have one or more sequence variations. Indeed, the method can be performed using a single aliquot if a sufficient number of sequence variations are analyzed.

In some embodiments, the method may additionally comprise sequencing 3-10 aliquots of the test DNA sample to produce, for each aliquot, sequence reads corresponding to 6 to 100 target regions. In some embodiments, method may comprise sequencing from about 3 to about 10 aliquots of the test DNA sample to produce, for each aliquot, sequence reads corresponding to about 6 to about 100 target regions and 8 to 50 control regions.

In some embodiments, the method may additionally comprise sequencing 3-10 aliquots of the test DNA sample to produce, for each aliquot, sequence reads corresponding to 2 to 100, 4 to 100, or 6 to 100 target regions. In some embodiments, method may comprise sequencing from about 3 to about 10 aliquots of the test DNA sample to produce, for each aliquot, sequence reads corresponding to about 2 to about 100, 4 to about 100, or 6 to about 100 target regions, and 8 to 50 control regions.

This method may comprise: (a) sequencing multiple aliquots of the test sample to produce, for each aliquot, sequence reads corresponding to two or more target regions that each have one or more sequence variations present within the patient's cancer; (b) for each aliquot, for each target region: i. determining the number of sequence reads that have the sequence variation; ii. determining the total number of sequence reads; and iii. comparing i. and ii. to one or more error probability distribution models for the sequence variation, wherein the one or more models are obtained from DNA that does not contain the sequence variation; and (c) integrating the collective results of step (b) to determine if there is cancer DNA in the test sample.

In some embodiments the method may comprise:
sequencing from about 3 to about 10 aliquots of the test DNA sample to produce, for each aliquot, sequence reads corresponding to from about 8 to about 100 target regions that each have one or more sequence variations present within the patient's cancer, wherein the cancer may be a solid tumor or a haematological cancer;
for each aliquot, for each target region: i. determining the number of sequence reads that have the sequence variation; ii. determining the total number of sequence reads; and iii. comparing i. and ii. to one or more error probability distribution models for the sequence variation, wherein the one or more probability distribution models are obtained in advance using a database of control DNA that does not contain the sequence variation; and (c) integrating the collective results of step (b) to determine if there is cancer DNA in the test sample.

In some embodiments the method may comprise:
sequencing from about 3 to about 10 aliquots of the test DNA sample to produce, for each aliquot, sequence reads corresponding to from about 6 to about 100 target regions that each have one or more sequence variations present within the patient's cancer and sequence reads corresponding to from about 8 to about 50 control regions, wherein the cancer may be a solid tumor or a haematological cancer;
for each aliquot, for each target region: i. determining the number of sequence reads that have the sequence variation; ii. determining the total number of sequence reads; and iii. comparing i. and ii. to one or more error probability distribution models for the sequence variation, wherein the one or more probability distribution models are obtained in advance using a database of control DNA that does not contain the sequence variation; and (c) integrating the collective results of step (b) to determine if there is cancer DNA in the test sample.

In some embodiments, the method can comprise: (a) measuring one or more aliquots of the test sample to produce, for each aliquot, a signal corresponding to two or more target regions that each have one or more sequence variations present within the patient's cancer and at least one control region; (b) for each aliquot, for each target region: i. determining the amount of signal from the sequence variation; ii. comparing the amount of signal to one or more error probability distribution models for the sequence variation, wherein the one or more models are obtained from DNA that does not contain the sequence variation; and iii). optionally, eliminating variants that are above a threshold in a statistically improbable number of aliquots; and (c) integrating the collective results of step (b) to determine if there is cancer DNA in the test sample.

In some embodiments, the measuring comprises sequencing one or more aliquots of the test sample, and the signal corresponding to two or more target regions comprises sequence reads. In some embodiments, determining the amount of signal from the sequence reads comprises determining the number of sequence reads that have the sequence variation. In some embodiments, the method further comprises determining the total number of sequence reads, wherein comparing the amount of signal comprises comparing the number of sequence reads that have the sequence variation and the total number of sequence reads to the one or more error probability distributions.

In some embodiments, the measuring can comprise quantitative PCR (qPCR) and the signal can be an intensity of fluorescence. qPCR is a technique used to amplify and quantify specific DNA sequences. It can measure the amount of a particular genetic sequence in a sample, and is often used in applications such as gene expression analysis, genetic testing, and pathogen detection. The basic principle of qPCR is to amplify a specific DNA sequence using polymerase chain reaction (PCR) and measure the amount of amplified product using fluorescent dyes. The fluorescence is directly proportional to the amount of amplified DNA, which allows for precise quantification of the target sequence. Thus, in some embodiments, measuring the one or more aliquots comprises measuring a level of fluorescence. In some embodiments, measuring a level of fluorescence comprises amplifying and quantifying a target region using (e.g.) qPCR. In some embodiments, comparing the amount of signal to one or more error probability distribution models comprises comparing the level of fluorescence with an error probability distribution model.

In some embodiments, the measuring can comprise digital PCR (dPCR) and the signal can be a count of the number of positive reactions in a plurality of wells, droplets, or other compartments, dPCR allows for the quantification of target regions with high precision and sensitivity. Unlike traditional PCR, which amplifies a sample and then uses gel electrophoresis or fluorescent dyes to measure the amount of amplified product, dPCR physically partitions a sample into thousands of tiny droplets, each of which acts as an individual PCR reaction. dPCR can accurately determine whether a target region is present by amplifying the target region and then counting the number of droplets that contain the amplified target region. Thus, in some embodiments, measuring the one or more aliquots comprises measuring a level of fluorescence in a plurality of compartments, such as wells or droplets. In some embodiments, measuring a level of fluorescence comprises amplifying and quantifying a target region using (e.g.) dPCR. In some embodiments, the number of positive compartments containing amplified target region is counted and compared to one or more error probability distributions. In some embodiments, the level of fluorescence in each compartment is compared with an error probability distribution to determine whether a compartment contains amplified target region.

In these embodiments, the different aliquots contain different aliquots (i.e., portions) of the same sample. As would be appreciated, different barcode sequences can be added to the different samples and the different samples can be pooled prior to sequencing.

### Flow Charts

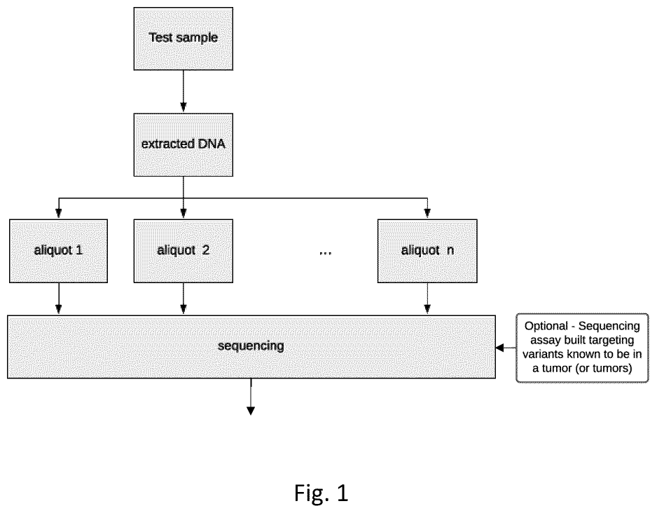

Some of the workflow for the present method is illustrated in the accompanying flow charts (FIGS. 1-10). These flow charts are believed to be largely self-explanatory. As shown in FIG. 1, an embodiment of the present method can begin by procuring a test sample, such as a sample of blood collected from a cancer patient. DNA may then be extracted from the sample and separated into one or more aliquots, which is then sequenced to generate a plurality of sequence reads for each aliquot. Optionally, a sequencing assay may be built targeting variants known to be in a tumor (or tumors).

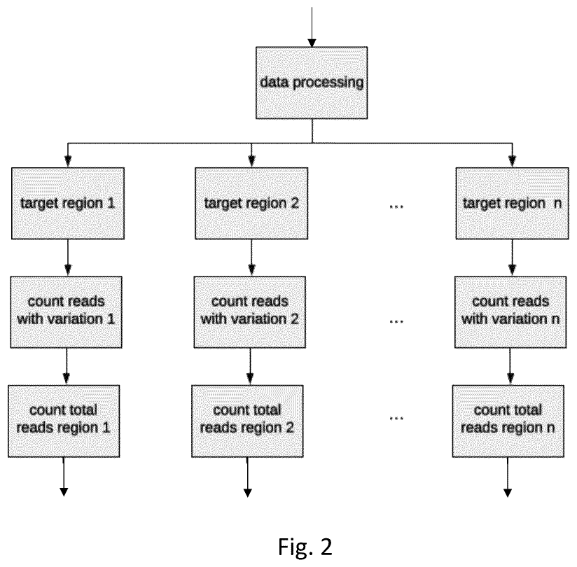

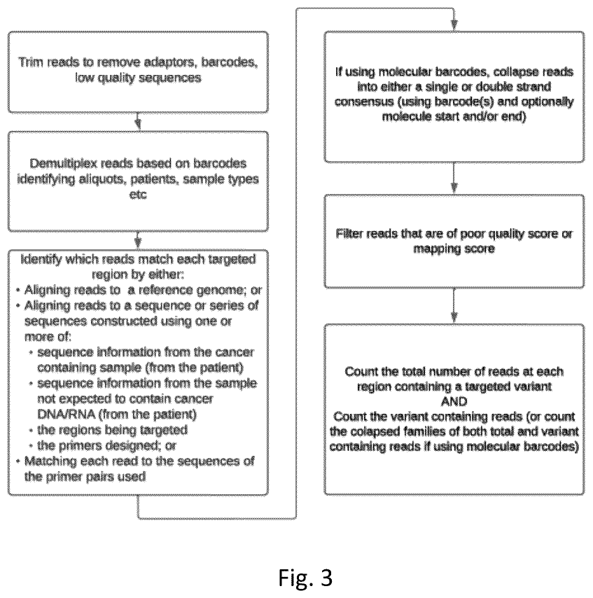

The embodiment of the present method continues in FIG. 2. The sequence reads for each aliquot may be processed computationally, e.g., by trimming, demultiplexing, aligning, matching, collapsing, filtering, or collapsing, as further described in FIG. 3. Typically, the processing will assign each of the sequence reads to one or more target regions that contains or is suspected of containing one or more sequence variations associated with the patient's cancer. The number of sequence reads containing the sequence variation (n) and total number of sequence reads (N) are then determined.

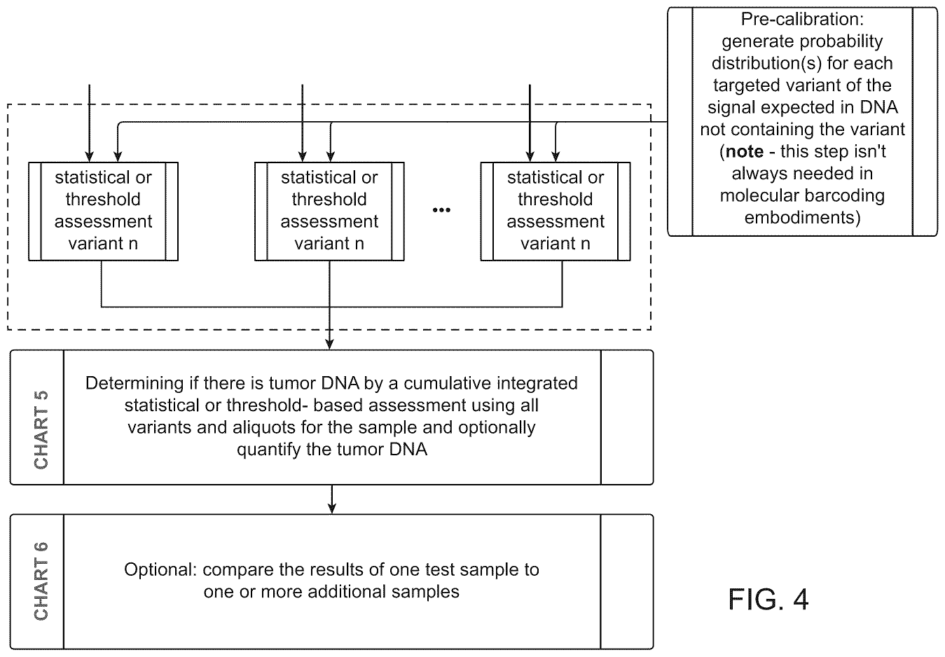

The embodiment of the present method continues in FIG. 4, in which an assessment is made for each variant in a target region, and in each aliquot, to determine whether the one or more sequence variations within a target region are present in the test sample.

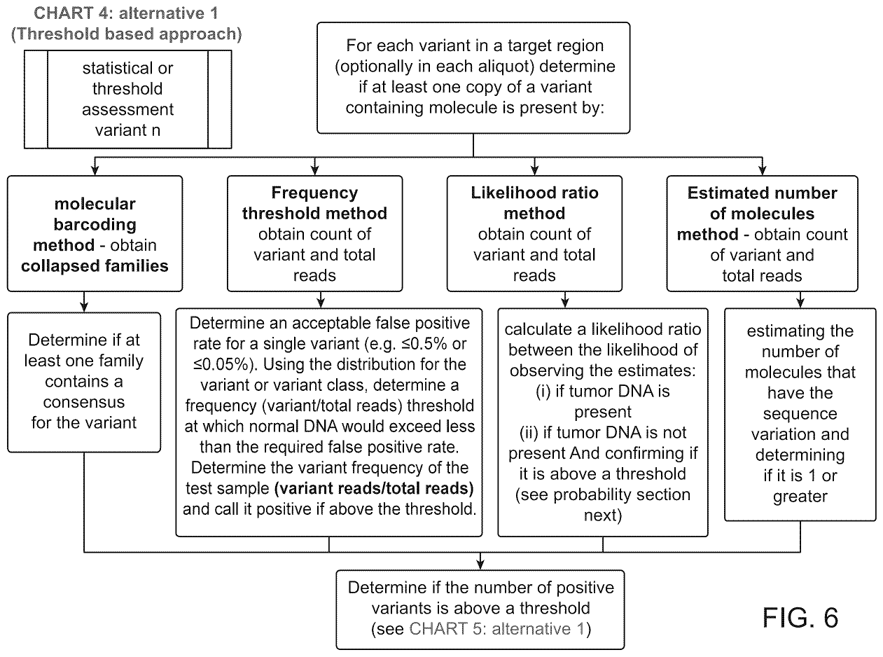

In some embodiments, the assessment is a threshold assessment in which each target region and each aliquot are scored and compared to a threshold to determine whether the one or more sequence variations are present in the sample. As shown in more detail in FIG. 6, a threshold assessment can include a molecular barcoding method in which aligned sequence reads in each target region and in each aliquot are collapsed into a consensus sequence. If at least one consensus sequence includes the one or more sequence variations, the one or more sequence variations are considered present in the sample. A threshold assessment may also include a frequency method in which an acceptable false positive rate (e.g., <0.5%, <0.05%) is selected. The variant frequency (n/N) of the target region and aliquot is then determined and compared to a threshold. A threshold assessment may also include a likelihood ratio method that calculates the likelihood of observing the variant frequency (i) if cancer DNA is present in the sample and (ii) if cancer DNA is not present and comparing to a threshold. Additionally, a threshold assessment may also include an estimated number of molecules method, wherein an estimate of the number of molecules that have the sequence variation is made and if this value is 1 or greater.

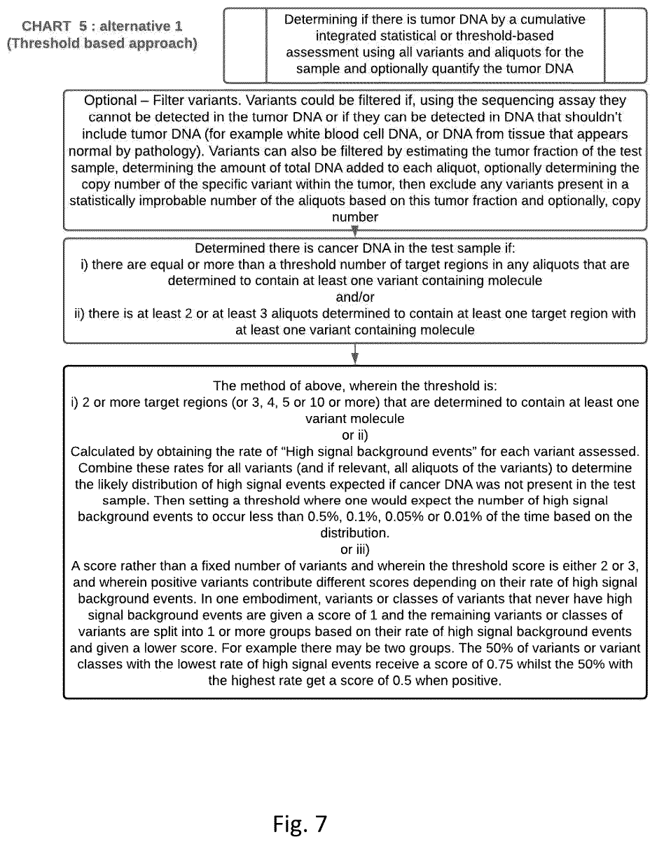

In any of these embodiments, cancer DNA may be considered present in the sample based on the plurality of assessments. For example, cancer DNA may be considered present in the sample if the number of target regions and aliquots having the one or more sequence variations exceeds a threshold number. As further described in FIG. 7, this determination can be made if there are equal or more than a threshold number of target regions in any aliquots that are determined to contain at least one sequence variation. In some embodiments, the threshold is 2 or more target regions, 3 or more target regions, 4 or more target regions, five or more target regions, or 10 or more target regions. In some embodiments, the threshold may be at least from 1 in 5, 1 in 6, 1 in 7, 1 in 8, 1 in 9, or 1 positive call in every 10 target regions tested. Preferably, there is at least 1 positive call in every 8 target regions tested. The threshold may also be determined by obtaining a rate of high signal background events for each sequence variation and determining the likely distribution of events expected if cancer DNA was not present in the test sample. In such cases, one could set a threshold where one would expect the number of high signal background events to occur less than 0.5%, 0.1%, 0.05%, or 0.01% of the time based on the distribution. The threshold assessment may also be made using a score rather than a fixed number of variants, wherein positive variants contribute scores depending on their rate of high signal background events, and wherein the score may be (e.g.) 2 or 3.

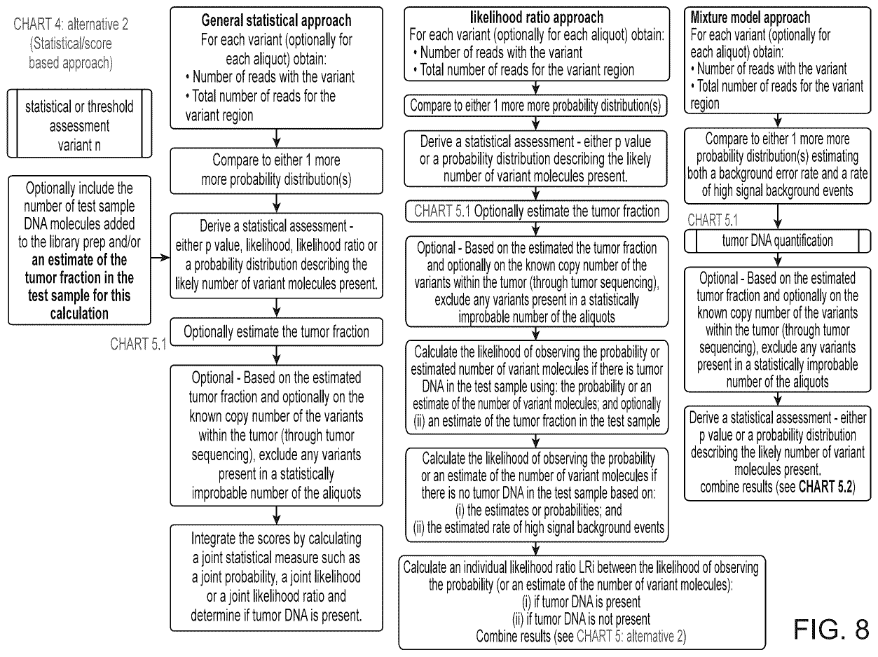

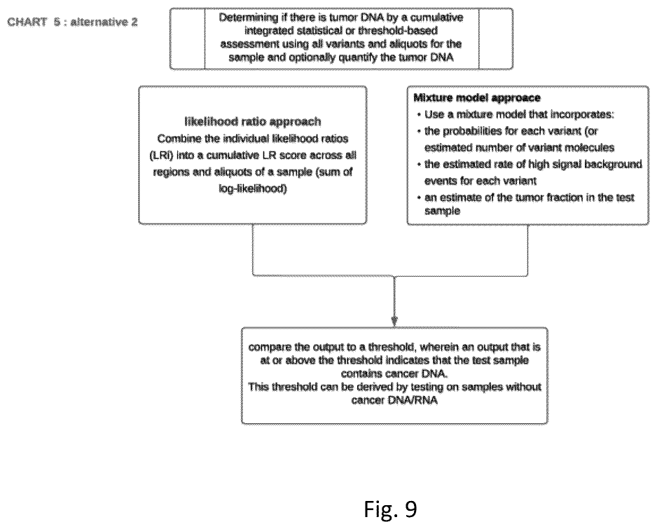

In some embodiments, the assessment is a statistical assessment. As shown in more detail in FIG. 8, a statistical assessment can include a general statistical approach in which n and N are compared to one or more probability distributions. A statistical assessment, e.g., a p-value, likelihood, likelihood ratio, or a probability distribution describing the likely number of variant molecules present, is generated to determine whether the one or more sequence variations are present in a target region. A statistical assessment may also include a likelihood ratio approach in which the likelihood of observing n sequence reads containing the one or more sequence variations in the test sample is determined if i) there is cancer DNA in the sample, and ii) there is not cancer DNA in the sample. These values may then be used to calculate a likelihood ratio to determine whether the one or more sequence variations in a target region are present in the sample, and the cumulative likelihood ratios may be combined to determine whether cancer DNA is present in the sample. A statistical assessment may also include a mixture model approach in which the n sequence reads are compared to a one or more probability distributions including both a background error rate and a rate of high signal background events. As further described in FIG. 9, in any of these embodiments, the method can further comprise determining whether there is cancer DNA within the sample based on the plurality of assessments. For example, this can include a joint statistical measure (such as a joint probability, joint likelihood, or joint likelihood ratio) integrating (e.g., summing, averaging) the results for each of the target regions and for each aliquot may then be calculated to determine whether cancer DNA is present in the sample.

Generally, embodiments of the disclosure can compare measurements of target regions (e.g., counts of sequence reads, signal intensity, numbers of PCR cycles before a signal intensity is reached, counts of positive reactions, etc.) with an error probability distribution model (i.e., a background error distribution model) to determine if the measurements are statistically significant. The background error distribution represents the variability of the assay when a sequence variation is not present in a target region. By comparing measurements to a background error distribution, it is possible to determine whether the measurements are within the range of normal variability or if they are significantly different and thus represent the presence of a sequence variation. For example, measurements can be compared by using a statistical test, such as a t-test or analysis of variance (ANOVA). These tests calculate a p-value, which represents a probability that the measurements are due to random chance rather than a true presence of a sequence variation. In some embodiments, a p-value of less than 0.05 is considered statistically significant, indicating that the results are unlikely to have occurred by chance. In some embodiments, error probability distribution models are determined for each assay, as the variability may differ depending on specific conditions. In some embodiments, the measurements may be compared with a binomial distribution or beta-binomial distribution, as described in further detail herein.

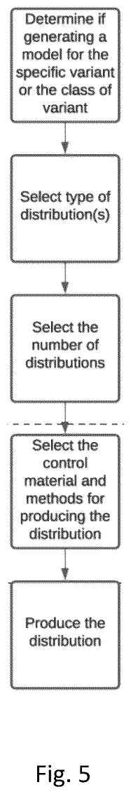

In some embodiments, a probability distribution for each targeted variant of the signal expected in DNA not containing the variant is generated (FIG. 5). The result is a plurality of assessments indicating whether a cancer-associated variant is present for each aliquot and target region.

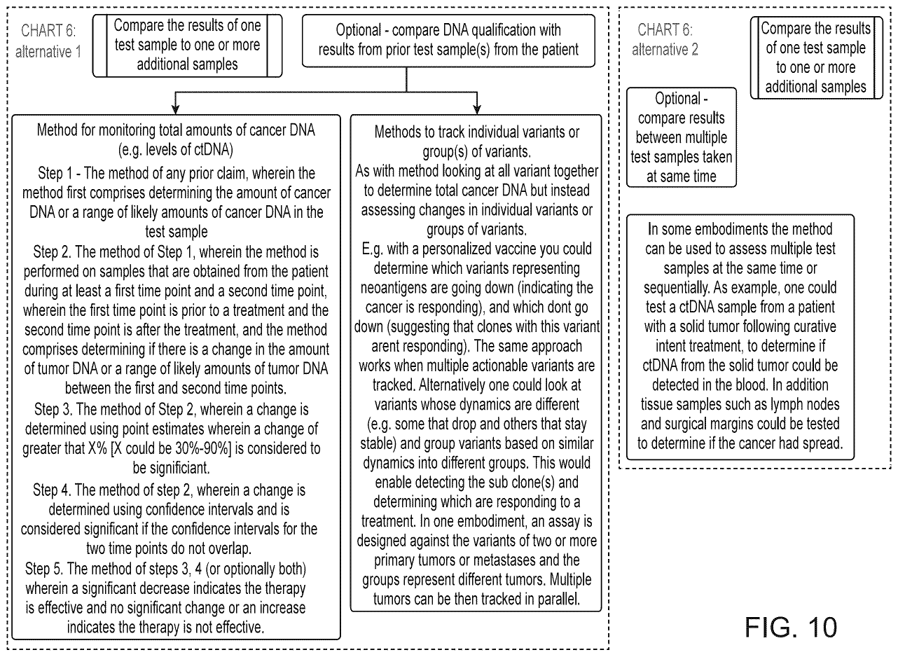

In some embodiments, the amount of cancer DNA within the sample may be quantified based on the determination of whether cancer DNA is present in the sample. Quantification may include an estimated variant allele fraction. In some embodiments, the estimated allele fraction can comprise a mean of the variant allele fraction for each variant and each aliquot in which it was determined that the one or more sequence variations was present. In some embodiments, the estimated variant allele fraction can comprise a mean of the variant allele fraction for each variant and each aliquot. This can be preferable in situations where variant levels are low and the results are stochastic, and therefore including evidence from all variants may result in a more realistic measure. As further described in FIG. 10, quantified cancer DNA may be compared to one or more additional samples, such as samples obtained from a patient during at least a first time point and a second time point, wherein the first time point is prior to a treatment and the second time point is after a treatment. Similarly, one could track individual variants or groups of variants across samples and time points. Each of these embodiments of the disclosure are described in more detail herein. Before describing the method in more detail, is noted that the present method can be used to detect cancer DNA from both solid tumors and haematological cancers. Therefore, when this claim uses the term “cancer”, the term refers to blood cancers and solid tumors. For solid tumor embodiments, the method may identify cancer DNA (or, more accurately, tumor DNA) in cfDNA (e.g., circulating cfDNA). For blood cancer embodiments, the method may identify cancer DNA in DNA extracted from cells taken from bone marrow, lymph node, or circulating white blood cells, or in cfDNA. For example, in blood cancer embodiments, one could take a bone marrow aspirate from an AML patient (pre treatment), find out the variants in their AML, then, following treatment, one could look at further bone marrow aspirates, cell free DNA or urine to determine if the patient still has cancer DNA.

In addition, the nucleic acid analyzed in the method may be DNA or RNA. The present disclosure is written describing embodiments that make use of DNA (specifically ctDNA). However the method should also work when one uses RNA (or cDNA) made from the same. In a preferred embodiment, the nucleic acid analyzed in the method is DNA.

In addition, while the present method is described in detail using examples that make use of “amplicon” sequencing, the present method may be readily applied to methods that make use of molecular barcodes or indexes, e.g., random sequences that are appended to the nucleic acid, pre-amplification. Molecular barcode sequences may vary widely in size and composition; the following references provide guidance for selecting sets of barcode sequences appropriate for particular embodiments: Casbon (Nuc. Acids Res. 2011, 22 e81), Brenner, U.S. Pat. No. 5,635,400; Brenner et al, Proc. Natl. Acad. Sci., 97:1665-1670 (2000); Shoemaker et al, Nature Genetics, 14:450-456 (1996); Morris et al, European patent publication 0799897A1; Wallace, U.S. Pat. No. 5,981,179 (the contents of which are each hereby incorporated by reference in their entireties); and the like. In particular embodiments, a barcode sequence may have a length in range of from 2 to 36 nucleotides, or from 6 to 30 nucleotides, or from 8 to 20 nucleotides. For example, the aliquot-based sequencing may be done on DNA that has been indexed, the number of molecules/the probability of a molecule being present can be estimated using index sequences in each aliquot.

It is noted that in the pre-calibration method shown in FIG. 5 the types and classes of variants may vary for which the error probability distributions are generated. For example, the specific variant may be analyzed within the context of its surrounding sequence. This can be achieved by sequencing the target region using DNA not expected to contain the variant (e.g. DNA from a healthy donor who is assumed to not have cancer) or by spiking in synthetic DNA/RNA for the target region that contains the wild type sequence and a barcode (outside of the variant region) enabling the separation of barcode and spike to the test reaction. In another example, the specific variant may be analyzed within the context of a class of variant. Classes of variants include: The same type of variant (e.g. An SNV such as A>T, an indel such as insertion of a TTTT, a doublet-base substitution such as CT>AA etc.); a transition or transversion; the single nucleotide variant and 1 to 5 bases either 3′, 5′ or both (e.g. A>T where the A has a 5′TTCA (TTCAA>TTCAT), or A>T where the A has a 5′ T and a 3′ G (TAG>TTG). Alternatively variants may be grouped into classes as above but where some or all of the bases 3′ and/or 5′ of the variant may be one of multiple bases as described by the IUPAC degenerate nucleotide codes. (e.g. A>T where the A has a 5′ K and a 3'S (KAS>KTS) (where K=G/T and S=C/G). In an alternative embodiment the local sequence context is explored by selecting a window of N 3′ and or 5′ bases around the variant of interest, where N is between 1 and 100, and extracting different sequence descriptors such as the base change at each location, the type of base change at each position (e.g., transition or transversion), the distance from a primer end, the distance from a repeat sequence and these are then combined together to predict a categorical error rate class (e.g. high, medium, low) or a numeric error rate value by using a heuristic combination score or a machine learning method (unsupervised or supervised). The method as one of the above, but where a penalty score is assigned in the form of a multiplicative factor to the estimated error rate of a variant in proximity of pre-defined sequence features, such as mono-nucleotide repeats, repeat regions, or similar. This analysis can be done by sequencing DNA not expected to contain the classes of variants (e.g. DNA from a healthy donor who is assumed to not have cancer). In this embodiment, enough regions must be targeted and sequenced so that each variant class is represented at least once (and ideally more e.g. 10 times or 50 times or 100 times).

Accordingly, with respect to the steps relating to determining the error probability distribution, in some embodiments the method comprises:
identifying at least one sequence variation (in each target region);
(ii) determining a class (i.e. type) of the at least one sequence variation; and
(iii) selecting, from one or more databases, an error probability distribution model corresponding to the class.

Different classes (or types) of sequence variation include SNPs, SNVs, indels, etc., as well as sequences immediately adjacent to the variant sequence itself. In some embodiments, the at least one sequence variation is a single nucleotide variation (SNV). In some embodiments, the class can comprise a sequence containing the variation, including but not limited to one or more nucleotide bases (for example from one to 3 nucleotide bases) immediately adjacent to the 5′ end of the sequence variation and/or one or more nucleotide bases (for example from one to 3 nucleotide bases) immediately adjacent to the 3′ end of the variation. In some embodiments, the class can comprise a sequence containing the variation, including but not limited to one nucleotide base immediately adjacent to the 5′ end of the sequence variation and one nucleotide base immediately adjacent to the 3′ end of the variation. In some embodiments, the class can comprise one or more ambiguous bases (e.g., IUPAC degenerate codes) indicating possible nucleotides for a position in the sequence. In some embodiments, an error probability distribution model is determined for each class. In these embodiments, the error probability distribution model may be determined by sequencing one or more control samples including a sequence containing the class. In some embodiments, the method further comprises determining whether the at least one sequence variation identified in step (i) is present in the test sample using the selected error probability distribution model. For further examples of different classes that may be used in this method and additional examples of motif-based methods for selecting error probability distributions, see also WO2019/241349 (the contents of which are hereby incorporated by reference).

In addition, the number and type of error probability distributions may vary. In some versions for each variant (or class) there is a single distribution for all errors. In other embodiments, there are multiple distributions separating the different types of error. In some embodiments there are two error distributions for each variant, one of which is for the “estimated background error rate”. These are typically sequencing error and PCR errors that happen later in library preparation (e.g. after the first few cycles of PCR). Then there are events that happen much less frequently but when they do, at much higher levels and typically at a similar level (in terms of variant allele fraction) to real variants in a sample. These “high signal background events” include things such as DNA damage and polymerase errors in the first few cycles of library preparation or pre-amplification. These can be captured by a second distribution (e.g. one binomial distribution for the estimated background error rate and one for the high signal background events). In some embodiments, a different distribution is used for the estimated background error rate and the high signal background events (e.g. a beta distribution for the estimated background error rate and a binomial distribution for the High signal background events). In some embodiments, high signal background events can be minimized by including an allele fraction cutoff (e.g., <0.01) for considering a given sequence variation. In some embodiments, a single distribution may account for one or more types of error. For example, the two shape parameters (α, β) in a beta-binomial distribution may be tuned to accommodate an estimated background error rate and High signal background events.

In some embodiments for each variant, the same variant class (e.g. 2 bp 3′ and 2 bp 5′) are used for both distributions. However as the two different distributions are sometimes the outcome of different error processes (e.g. DNA damage and PCR error) in some embodiments, for each variant, a different variant class is used for the two distributions.

The control material and methods for producing the distribution or distributions may also vary. For example, the probability distribution can be generated in the same library preparation and run as the test sample, in advance using control DNA, or in advance then adjusted using all bases other than the bases expected to contain variants when assessing the test sample(s). Preferably, the probability distribution is generated in advance using a database of control DNA that contains the class of sequence variation. Preferably, the probability distribution is generated in advance using a database of control DNA that contains the class of sequence variation and optionally is derived from subjects who are assumed to not have cancer.

In all cases the same sequencing process (including library prep, sequencer) and optimally the same sample type and extraction method (e.g. cfDNA extracted from blood drawn into a cfDNA blood collection tube) should be used to generate the model(s). The assay may be run multiple times, preferably wherein the preparation and sequencing steps are the same.

In some cases a different model is produced for a range of different DNA inputs and the test sample is analysed using the model with the best matched DNA input. For example, a maximum, minimum and median DNA input for each aliquot can be defined then a distribution or distributions obtained for all three for all the classes of variants tested for. In some embodiments, each aliquot comprises from about 100 to about 10000 amplifiable copies of the genome (prior to any amplification). The about 100 to about 10000 amplifiable copies of the genome in each aliquot are in the form of fragments, such as cfDNA fragments. That is, for each section of the genome, there may be at least 100 to 10000 amplifiable copies in the form of amplifiable fragments, such as cfDNA fragments.

Whether or not DNA fragments (such as cfDNA fragments) are amplifiable or not may be determined by the length of the fragments, based on the design (i.e. length) of the primers used for amplification, and the length of the intended amplicon (i.e. how far apart (e.g., distance, in number of nucleotides) the pair of primers are when aligned to the patient genome). For example, amplifiable cfDNA fragments may be at least 100 base pairs in length. As used herein, the number of amplifiable copies is equivalent to the number of input molecules. When a test sample is assessed it is compared to the distribution whose DNA input is the closest match.

In some embodiments, each aliquot comprises from about 10 ng to about 100 ng of DNA fragments (e.g. cfDNA fragments) (or in the case of embodiments using only one aliquot, the amount of DNA fragments (such as cfDNA fragments) may be from about 10 ng to about 100 ng). In some embodiments, the aliquot (or test sample, as the case may be) comprises at least 10 ng, at least 20 ng, at least 30 ng, at least 40n, at least 50 ng, at least 60 ng, at least 70 ng, at least 80 ng, at least 90 ng, or at least 100 ng of DNA. In some embodiments, the test sample comprises 66 ng of DNA.

Optimally there would be tens, hundreds or thousands of samples tested to build the error probability distribution model. Preferably, at least about 50 samples are tested to build the model.

The distribution can be stored in a database and/or be downloaded from a public database.

Preferably, the database comprises data from at least about 50 samples taken from healthy donors (e.g. a donor who is assumed to not have cancer).

In some embodiments, (e.g., as shown in FIG. 8) the amount of cancer DNA may be quantified using the method. In these embodiments, one may determine the amount of cancer DNA in the test sample, a range of likely amounts in the test sample, or an estimated tumor fraction using one or a combination of: a mean or median variant allele fraction (across the variants and aliquots), a corrected mean or median variant allele fraction (generated by subtracting a previously pre-determined offset or baseline error rate), maximum likelihood (testing a range of levels and determine the most likely), estimating tumor fraction: a grid based or an expectation maximisation search method to select the tumor fraction giving the maximum likelihood, Bayesian posterior or summing the number of estimated variant molecules for each variant (and optionally each aliquot). In another embodiment the amount of cancer DNA may be determined by counting the number of variant positive target regions (target region above a threshold) in each aliquot and comparing this against the total number of target regions multiplied by aliquots and quantifying the mean number of variant containing target sequences per target region per aliquot by applying a Poisson correction to the fraction of the positive results. In some embodiments, the rate of high signal background events estimated for the entire set of variants may also be used in the Poisson correction in order to give more accurate quantification.

### General Methodology

In some embodiments, the method comprises: (a) sequencing multiple aliquots of the test sample to produce, for each aliquot, sequence reads corresponding to two or more target regions that each have one or more sequence variations present within the patient's cancer; (b) for each aliquot, for each target region: deriving an estimate of the number of molecules that have the sequence variation, calculating the probability that there is at least one molecule that has the sequence variation, or determining if the frequency of sequence reads of (a) that have the sequence variation compared to the total number of sequence reads is above a threshold; and (c) determining if there is cancer DNA in the test sample using estimates, or probabilities or frequencies of step (b). In some embodiment, steps (b) may be done by a thresholding approach, described below and, in alternative embodiments, step (a) can be done without aliquoting as long as there are a sufficient number of target regions.

In some embodiments, for each aliquot and target region, the number of molecules that have the sequence variation in the test sample or the probability that there is at least one molecule that has the sequence variation is estimated (b) using: (i) the number of sequence reads of (a) that have the sequence variation; (ii) the total number of sequence reads of (a); and (iii) the estimated background error rate for the sequence variation. The background error rate of (iii) may be expressed by an error probability distribution. In addition, the probability that there is at least one molecule that has the sequence variation is estimated using the number of molecules inputted into each aliquot of (a). This allows adjustment of the method depending on the number of DNA molecules determined to be present in each aliquot, since this can vary greatly. The estimated background error rate of (iii) is estimated by any convenient method, e.g., from prior sequencing reactions or publicly available information, e.g., from prior sequencing reactions, adjusted using data for control bases obtained in step (a), and/or from the current sequencing reaction, excluding the variant of interest. For example, the estimated background error rate of (iii) may be estimated by analysis of control sequencing reads produced in step (a).

In any embodiment, the background error rate can be estimated using a probability distribution. In some embodiments, there may be two distributions of the same family or type (e.g. 2 binomial distributions) or, if two different families or types of distribution are used, there may be one distribution for the background error rate and another for the estimated rate of high signal background events. As noted above, in any embodiment, the estimate is a probability distribution over the number of variant molecules present.

In any embodiment, (c) may be done by calculating a likelihood ratio between the likelihood of observing the estimates in (b) in samples: (i) if cancer DNA is present (ii) if cancer DNA is not present. Along similar lines, in any embodiment (c) may be done by calculating a likelihood ratio (LRi) between the likelihood of observing the estimates in (b) for each target region and aliquot: (i) if cancer DNA is present (ii) if cancer DNA is not present. In these embodiments, the individual likelihood ratios LRi may be combined into a cumulative LR score (product of LRi equivalent to sum of log-likelihoods) across all regions and aliquots of a sample. In these embodiments, the likelihood of observing the estimates of (b) if there is cancer DNA in the test sample may be calculated based on: (i) the estimates or probabilities of step (b); and optionally (ii) an estimate of the cancer DNA fraction in the test sample. Likewise, the likelihood of observing the estimates of (b) if there is no cancer DNA in the test sample may be calculated based on: (i) the estimates or probabilities of step (b); and (ii) the estimated rate of high signal background events

In any embodiment, step (c) may be calculated by using a mixture model incorporating: (i) the estimates or probabilities of step (b); and (ii) the estimated rate of high signal background events; and optionally (iii) an estimate of the cancer DNA fraction in the test sample. Similarly, the mixture model may be used to calculate a likelihood ratio between the likelihood of observing the estimates in (b) in samples: (i) if cancer DNA is present (ii) if cancer DNA is not present. For example, in some cases, step (c) may further comprise comparing the likelihood ratio generated from a mixture model to a threshold, wherein an output that is at or above the threshold indicates that the test sample contains cancer DNA.

The threshold may be determined by running at least 10 or at least 100 or at least 1000, or at least 10,000 samples comprising non-cancerous DNA (or at least are not known to have cancer DNA) through the assay and selecting a threshold above the signal identified in the control samples or a threshold such that the false positive rate as determined using the control samples is estimated to be 1% or below, 0.1% or below or 0.01% or below. The samples which are run may be from the same patient or they may be from different patients. For example, running 200 samples may involve taking a sample from 20 healthy donors (assumed to not have cancer) and running 10 assays per patient to reach 200 samples. For each control sample the likelihood ratio analysis may be applied to give an overall likelihood ratio for a healthy patient. Calculating the likelihood ratio for all the samples which have been run results in a range of likelihood ratios for a healthy patient and the threshold can be set somewhere above the highest likelihood ratio. This threshold may calculated from a pool of healthy donors in advance and therefore does not change on a patient-by-patient basis. As would be apparent, the method may further comprise identifying the patient as having cancer cells if the result is at or above the threshold and, for example, administering a therapy to the patient. In these embodiments, the patient may have previously undergone a first therapy. In these cases, the method comprises administering to the patient a second therapy that is different to the first therapy.

In any embodiment, the method may further comprise determining the amount of cancer DNA or a range of likely amounts of cancer DNA in the test sample based on the estimates of step (b). This step may be done by, e.g., (i) calculating the mean or median variant allele fraction; (ii) maximum likelihood analysis; (iii) Bayesian posterior analysis; (iv) by counting the number of estimated mutant molecules for each variant and each aliquot or (v) by counting the number of variant positive target regions in each aliquot and comparing this against the total number of target regions multiplied by aliquots and quantifying the mean number of variant containing target sequences per target region per aliquot by applying a Poisson correction to the fraction of the positive results. This type of analysis has been done to calculate the number of starting molecules in digital PCR and can be adapted therefrom. In some embodiments, the variant allele fraction for a test sample may be determined using one or more probability distributions that model (e.g.) the background error rate and the rate of high signal background events. In such embodiments, an initial variant allele fraction for each variant is adjusted by considering the probability of observing a certain number of sequence reads within a target region containing the variant (e.g., 0, 1, 2, 3, 4, 5 or more) given the number of input molecules before amplification, the expected error, and the total number of sequence reads in the target region. The mean or median value for the set of corrected variant allele fractions may then be determined to identify a variant allele fraction for the sample, i.e., the cancer allele fraction. In another embodiment, only a subset of variants may be used to calculate the mean or median variant allele fraction, e.g. those variants which are nearest to a mean variant allele fraction, less than a threshold value based on the number of variants expected, or variants within positive target regions. In another embodiment, all variants are used to calculate the mean or median variant allele fraction.

In any embodiment, the method may be performed on samples that are obtained from the patient during at least a first time point and a second time point, wherein the first time point is prior to a treatment and the second time point is after the treatment, and the method comprises determining if there is a change in the amount of cancer DNA or a range of likely amounts of cancer DNA between the first and second time points. In any embodiment, further samples may be obtained at additional time points, for example wherein additional samples are taken after the second time point on a monthly, bimonthly, quarterly, or annual schedule. This change may be determined using point estimates, confidence intervals or both, and wherein a significant (e.g. a statistically significant) decrease indicates the therapy is effective and no significant (e.g. a statistically significant) change or increase indicates the therapy is not effective. In these cases, a change of at least two-fold, at least four-fold, at least six-fold, at least eight-fold or at least ten-fold may be considered significant (e.g. statistically significant). In these cases, a change of at least 20%, at least 30%, at least 50%, at least 70% or at least 90% may be considered significant (e.g. statistically significant). In some embodiments a change is considered significant (e.g. statistically significant) if the change is greater than a threshold such as 50% and the confidence intervals when quantifying cancer DNA for the first and second time point do not overlap. In these embodiments, a significant (e.g. a statistically significant) decrease indicates the therapy is effective and no significant (e.g. a statistically significant) change or increase indicates the therapy is not effective. In any embodiment, the percentage change may be considered significant (e.g. statistically significant) if it is above the LOD (or above an uncertainty threshold for the LOD) for the assay, patient population, or sample. In any embodiment, the percentage change may be considered significant (e.g. statistically significant) if it is above the LOQ (or above an uncertainty threshold for the LOQ) for the assay, patient population, or sample. In an embodiment where at least two samples are taken from a patient, and at least one of the two samples is above the LOQ for the assay, patient population, or sample, a change in the amount of cancer DNA between the two samples of at least 20% may be considered significant (e.g. statistically significant). In an embodiment where at least two samples are taken from a patient, and at least one of the two samples is above the LOQ for the assay, patient population, or sample, a change in the amount of cancer DNA between the two samples of at least 30% or at least 50% may be considered significant (e.g. statistically significant). In an embodiment where at least two samples are taken from a patient, and at least one of the two samples is above the LOD for the assay, patient population, or sample, a change in the amount of cancer DNA between the two samples of at least 20% may be considered significant (e.g. statistically significant). Statistically significant refers to a claim that a result from data generated by testing or experimentation is not likely to occur randomly or by chance, but is instead likely to be attributable to a specific cause. The degree of statistical significance can be varied (e.g., p<0.05, <0.01, <0.001) depending on an acceptable number of false positives.

In any embodiment, sequence variations that are identified in a statistically improbable number of the aliquots based on the estimated cancer DNA fraction, the number of DNA molecules added to each aliquot and optionally the number of times each variant is represented in an individual cancer cell (which may be determined through copy number analysis) are excluded from the results of step (b) prior to step (c). In any embodiment, step (a) may comprises sequencing at least three aliquots, e.g., 3, 4, 5, 6, 7, 8, 9, 10, 11 or 12 or more aliquots.

In some cases, if a variant is amplified in a cancer cell, then it may be expected to be in all aliquots. As such, this part of the method can be further improved by inputting the copy number of each variant in a cancer cell and using this to estimate the likely number of aliquots the should be above a threshold for each variant.

In some embodiments, step (a) may also comprise sequencing positive and or negative control samples which may include at least one of: cancer DNA from an aspirate, biopsy or surgery sample coming from the same patient, buffy coat DNA, buccal swab DNA, whole blood DNA, adjacent non-cancerous DNA, i.e., tissue that is adjacent to a tumor that appears non-cancerous or as reference DNA. The sequencing of these control samples may be performed at the same time as the test sample or it may be performed before or after sequencing the test sample. In preferred embodiments, the negative control is buffy coat DNA, which is sequenced at the same time as the test sample. In preferred embodiments, the positive control is cancer DNA taken from a biopsy from the same patient which is sequenced before the test sample and may be run as a single sample, as opposed to aliquots. Another preferred embodiment uses a commercially available blood product from a healthy donor (assumed to not have cancer) as a negative control sample, which is sequenced before the test sample and may be run as a single sample, as opposed to aliquots.

In any embodiment, variants that are not detected in the cancer DNA are excluded. In addition or separately, variants that are detected in the buffy coat, buccal swab, adjacent non-cancerous or whole blood, and/or other negative control may be excluded as they are likely to not be tumor specific. In some embodiments, variants that are detected in both cancer DNA and a control sample may be included if the frequency of the variant in a plasma sample is significantly higher (e.g., >10×, >100×, >1000×) than the frequency of the variant in a control sample, such as a buffy coat sample. In such cases, the large quantity of cancer DNA in a plasma sample may “bleed through” into the buffy coat sample and so should not be excluded.

In any embodiment the two or more target regions is at least 2, at least 4, at least 10, at least 20, at least 50, at least 100, at least 500, at least 1000 or at least 5,000 target regions. In many embodiments, 2-200, e.g., 6-100, target regions may be examined. The sequence variations of step (a) may be independently single nucleotide variants, indels, doublet-base substitutions (DBSs), transpositions, rearrangements, variable number tandem repeats, short tandem repeats or a viral genome (such as HPV) integrated into the patients genome.

In some embodiments, the variants may be epigenetic variants rather than sequence variants such as 5-methylcytosine (5mC) or 5-hydrossymethylcytosine. In certain embodiments sequence variants and epigenetic variants (e.g. sequence variants) are selected when 2 or more are present less than 10 bp apart, less than 50 bp apart or less than 100 bp apart.

As noted above the sequence variations analyzed in the method are pre-identified sequence variations. For example, the sequence variations may be identified by sequencing a sample of: (i) DNA or RNA isolated from a tissue biopsy that comprises cancer cells, (ii) DNA or RNA isolated from a cancer tissue obtained at surgery that comprises cancer cells or (iii) sequencing cell-free DNA or RNA or (iv) DNA or RNA isolated from circulating cancer cells, wherein the sample is from the same patient, e.g., prior to any treatment. In preferred embodiments, the entire exome of cancer DNA from a tissue biopsy or other surgical sample is sequenced. For blood cancers, the sequence variations may be identified by sequencing a sample of DNA or RNA from bone marrow, circulating blood cells or lymph node, for example. In some embodiments both DNA and RNA are sequenced and the variants identified in each combined. These sequence variations may be identified by sequencing the whole genome or by sequencing one or more of the whole exome, Genes frequently mutated in cancer (e.g. those in the COSMIC-Cancer Gene Census), the mitochondrial genome, Regions of common structural rearrangements (e.g. common gene fusions or the edges of common amplifications such as MYC), Regions of common amplification, Regions of common rearrangements (e.g. Chromothripsis), Regions of common localized hypermutation (e.g. Kataegis) or a region of the genome identified to typically contain sufficient numbers of mutations in the cancer type of interest that over 80% or 90% or 95% of the target patient population will have sufficient mutations identified to reach the required sensitivity (wherein the required sensitivity is pre-determined, as is the number of variants required to meet this sensitivity and this is compared to the rate of mutations per Megabase (Mb) and the variability between patients in the cancer type of in interest in order to determine the number of Mb of the genome to target).

For example, the sequence variations may be identified by sequencing a test sample of: (i) DNA or RNA isolated from a tissue biopsy that comprises cancer cells, (ii) DNA or RNA isolated from a cancer tissue obtained at surgery that comprises cancer cells, (iii) sequencing cell-free DNA or RNA or (iv) DNA or RNA isolated from circulating cancer cells, wherein the sample is from the same patient, e.g., prior to any treatment. A control sample of non-cancerous DNA or RNA is sequenced, for example buccal swab DNA, whole blood DNA, adjacent non-cancerous DNA, i.e. from tissue that is adjacent to a tumor that appears normal, and compared to the test sample. The sequencing of these control samples may be performed at the same time as the test sample or it may be performed before or after sequencing the test sample. Sequence variants that are detected in the test samples (cancer DNA) and not the control samples (non-cancerous DNA) may be selected to progress to primer design as they are likely to be tumor specific. Variants that are detected in the control samples (non-cancerous DNA) may be excluded as they are likely to not be cancer specific.

In some embodiments, copy number gain or amplification for a sequence variation is determined from (i) DNA or RNA isolated from a tissue biopsy that comprises cancer cells, (ii) DNA or RNA isolated from a cancer tissue obtained at surgery that comprises cancer cells, (iii) sequencing cell-free DNA or RNA or (iv) DNA or RNA isolated from circulating cancer cells, wherein the sample is from the same patient, e.g., prior to any treatment. Copy number gain or amplification can be determined using a read depth approach in which a non-overlapping sliding window is used to count the number of sequence reads that are mapped to a genomic region overlapping the window. Regions with a significant increase in read depth (more than expected according to typical background error associated with sequences) may be further analyzed to identify copy number. Alternately, a paired-end approach may be used in which copy number variations are detected based on distances between mapped paired sequence reads. Sequence reads may also be assembled de novo and the resulting assembled contiguous sequences may be aligned to the reference genome to identify copy number variation.

In some embodiments, viral sequences are targeted in order to identify those that have integrated into the human genome and where they have integrated. In some embodiments either the whole genome or specific regions of the genome are assessed for epigenetic changes for example by Whole-Genome Bisulfite Sequencing, TET-assisted pyridine borane sequencing, Enzymatic methyl-sequencing, Reduced representation of bisulfite sequencing, Methylated DNA immunoprecipitation sequencing or Target bisulfite sequencing. Both epigenetic and genetic changes can also be identified by array. In some embodiments, an assay utilising either methylation changes and/or sequence variants is performed as an assay for early detection of cancer through the identification of these changes in ctDNA. In such an embodiment, when a patient is identified as likely to have ctDNA and therefore cancer, the epigenetic and/or sequence variants that are present in the patients ctDNA sample are identified and selected for targeting.

Hotspots could also be sequenced. Alternatively, the sequence variations may be identified by RNA-seq and optionally wherein RNA selection/depletion such as PolyA selection or Ribosomal RNA depletion is used to target specific types of RNA.

In some embodiments, a plurality of candidate sequence variations are first identified and then certain sequence variations may be selected. In some embodiments, the variations may be ranked and then the “best” variations may be selected, variants may be filtered removing any that are not optimal for tracking or variants may be first filtered then ranked. In some embodiments, the sequence variations are filtered, scored or ranked based on one or more of: clonality, or allele fraction within the cancer sample, wherein variants present throughout the tumor are preferred. In some embodiments, clonality may be determined as a function of allele fraction. For example, clonality may comprise the allele fraction multiplied by the probability of the variant being a somatic variation. Optionally, this determination may be corrected for based on a detected copy number of the variant. However, in most cases, this determination will be equivalent to the allele fraction; mappability, wherein variants whose reads are hard to map based on attempted alignment of any predicted PCR amplicons designed to amplify the region or presence within pre-annotated black-lister regions, overlapping repeat and homopolymer region annotations should be avoided; estimated background error rate, wherein variants that have high error rate should are penalized or filtered; estimated rate of high signal background events wherein bases with low rates are preferential; distance from another selected variant. In some embodiments, the variants should be spaced evenly throughout the genome and not clustered together for example, there no more than 10% of all variants on any chromosome, or any chromosome arm, or any 1 Mb region. This is to prevent loss of a region of the genome (e.g. through loss of a chromosome arm during evolution) causing many variants no longer to be present for tracking. In another embodiment, if two variants are close enough to be targeted in a single sequencing read and present on the same chromosome, such variants are preferred; avoidance of certain target regions known to not be optimal for tracking purposes (e.g., by empirical evidence); predictive ability to sequence; presence within a region of copy number gain or amplification wherein variants present in multiple copies in a single cancer cell are preferred; proximity of any germ line variants which may be used for enriching the mutant allele; likelihood of being somatic, or likelihood of being somatic but not being from the cancer sample, such as being clonal hematopoiesis of indeterminate potential (CHIP). For example, cancer signatures (as described in more detail below) may be used to determine whether a variant is a somatic change specific to the cancer rather than either artefact, germline, or CHIP; presence on a region frequently lost in the cancer type being tested wherein avoiding such regions is preferred; likelihood of variant being a common SNP/polymorphism; likelihood of variant being artefactual occurring from specific protocol/sequencing method/capture kit. This includes through prevalence of variant in current and/or previous reaction/sequencing batch and variant profile matching that of known FFPE/other errors.

Whether a variant has a functional effect in cancer. For example, non-synonymous mutations predicted to be deleterious or probably damaging by variant effect predictor software (e.g., SIFT, PolyPhen2) may be prioritized for inclusion in an assay as these variants are more likely to be real somatic changes. However, in some embodiments, variants with functional effects are instead discounted as these variants are often targeted by cancer therapies. Including these variants may result in a negative ctDNA call, as other subclonal variants that have survived the therapy may lack that change.

In some embodiments, all or a combination of these factors are scored, the variants are ranked by the score, and then selected. For example, a variant that is clonal, mappable, has low error rate and is somatic (rather than germline) would score higher than a variant lacking those characteristics. In another example, a variant that is clonal, is present in multiple copies in a single cancer cell, is not in a region frequently lost in the cancer type being tested and is not likely to be an artefact occurring from specific protocol, would score higher than a variant lacking those characteristics. In another example, a variant that has a predictive ability to sequence, is clonal, has a low estimated rate of high signal background events and is somatic (rather than germline) would score higher than a variant lacking those characteristics.

In some embodiments, the combination comprises (i), (v), (viii) and (x)

In some embodiments, the combination comprises (ii), (v), (viii) and (x)

In some embodiments, the combination comprises (v), (vii) and (xi)

In some embodiments, the combination comprises (i), (iii), (v), (ix), (x), (xi) and (xii). In some embodiments regions of the genome are ranked rather than specific variants. In such an embodiment the genome may be divided into overlapping or non-overlapping windows. The windows can for example be 10 bp or 50 bp or 100 bp in length and these windows can overlap by 5 bp, 25 bp, 50 bp or not at all. As would be apparent to someone skilled in the art, the window should be smaller than the typical length of DNA from the test sample and shorter than the sequencing read length of the intended sequencing platform. Therefore with high molecular weight DNA and long read sequencers, the window could be 100, or 1000 or 10,000 bp as example. With Illumina® sequencers and cfDNA the windows should always be less than 160 bp (the typical length of cfDNA). In a preferred embodiment the window is between 20 and 100 by with an overlap that is half the length of the full window. Following the scoring of each variant, a score for each region is generated by combining the scores of all variants within the region, and optionally combining this with a score or scores for region specific features which may include mappability, predictive ability to sequence and presence within a region of copy number gain or amplification. In such an embodiment, the regions can be ranked and the best regions selected and an assay is designed to target these regions. An advantage of such a method is that it gives weight to regions of the genome where information may be obtained from multiple variants from a single molecule of test DNA (when the variants are is cis on the same chromosome) and simply getting more information from targeting a single region when the variants are in the same genomic region but are in trans i.e. on the other chromosomes. Once the variants are scored and ranked then PCR primers are designed.

In some embodiments, indels are prioritized for inclusion. Such variants are preferable as it is highly unlikely that observing these variants is due to error, resulting in a boost of sensitivity. Accordingly, in some embodiments, a sequence variation can comprise an indel of, e.g., 1-5, 1-10, 1-15, or 1-20 nucleotides in length (either an insertion of that number of nucleotides or a deletion of that number of nucleotides). In such embodiments, indels of 1-5 nucleotides are preferred as these are more likely to be observed in a tumor sample. In other embodiments, a sequence variation can comprise an indel of 1 or more, 2 or more, 3 or more, 4 or more, 5 or more, 6 or more, 7 or more, 8 or more, 9 or more, 10 or more, or 20 or more nucleotides in length. In some embodiments, a maximum indel length is 20 nucleotides as longer changes may affect the accuracy of sequence alignment. Depending on the type of sequencing technology used, the background error rate associated with indels may be very low. Accordingly, observing an indel in a target region may provide a relatively large amount of evidence that the indel (and thus, cancer DNA) is present in the test sample. Additionally, in embodiments that measure a level of fluorescence (such as qPCR, dPCR, and the like), indels are ideal amplification targets because a primer will better anneal to only sequence containing that indel, improving amplification relative to wild-type sequence and resulting in a higher fidelity signal.

In some embodiments, different combinations of PCR primer pairs (forward and reverse) are designed to target the plurality of candidate sequence variations or regions identified and these are selected, scored, filtered out or ranked in order to identify one single best primer pair for each of the variations or regions based on features which may include:
i. presence of repetitive region within the primer sequence (e.g., avoid homopolymer regions of >=6 nucleotides);
ii. presence of known Single Nucleotide Polymorphisms within primer sequence (wherein this is either avoided or the tumor sequencing is used to confirm is the SNP is present);
iii. predicted formation of unintended PCR products that are likely to be sequenceable as they are produced using 1 forward and one reverse primer based on in silico PCR and/or local alignment and/or 3′-based alignment of primers to primers and/or or primers to amplicon regions (wherein there is a high penalty for such primer combinations);
iv. as in iii), but the predicted formation of unintended PCR products that are likely to be unsequenceable (because they are either made with 2 forward primers or 2 reverse primers and such products would not allow sequencing as they would not contain both required sequencer adaptors) (wherein there is a low penalty for such primer combinations compared to iii));
v. total amplicon size in nucleotides;
vi. position of the variant within the resulting amplicon (e.g., prefer primers where the variant will be positioned approximately near the center of the amplicon as opposed to the edges);
vii. primer length, favoring relatively short primer sequences of ~20 nucleotides targeting the target region, as a shorter primer length will result in additional target region sequence in the resulting amplicon;
viii. melting temperature;
ix. number of times the predicted PCR product aligns to regions of the genome beyond the expected target (ranking score may be based on multiple mapping);
x. number of times the primer sequences align to regions of the genome other than the intended target;
xi. number of times there is alignment of a primer pair constituted by a forward and a reverse primer other than the intended one in close proximity (i.e. less than 50, less than 100 or less than 150 nucleotides, based on a pre-defined threshold);
xii. combined score of all variants present within the target amplicon;
xiii. avoidance of primer sequences within certain target regions known to not be optimal for amplifiability (e.g., with previously collected empirical evidence).

In some embodiments, the primers are filtered based on some or all of these features when a score is above a threshold. In some embodiments a composite scoring based on a linear or polynomial combination of some or all of the features is used to select the optimum multiplex. In some embodiments, a large number of variants are selected from a cancer DNA containing sample or cell line and a plurality of multiplex PCR panels are designed against these variants. A dilution series of the cancer DNA into non-cancerous DNA is generated then the plurality of multiplex PCR assays are used to generate sequencing libraries from the DNA. The process is optimally repeated with at least 10 or at least 100 samples. Some or all of the primer features along with the sequencing signal are inputted into a machine learning system or a neural network in order to determine the optimal combination of primers for detecting cancer DNA in a test sample. For example, such a machine learning system could be trained based on features derived from a set of primers with corresponding empirical evidence of amplifiability, efficiency, etc. Previously unseen primer sequences could then be provided to the machine learning system which would score and rank these sequences (e.g., on a scale of 0 to 1). Similarly, an unsupervised machine learning method could be used to classify primers into one or more clusters having different properties. The primers are all checked together (in case of primer/dimer formation and other unintended interactions between primers of different primer pairs) to then design the best multiplex PCR reaction (with the variants selected based on the score and rank).

In some embodiments, the library preparation reaction may produce a sequencing library that includes both amplified copies of the target and control regions of interest and other unintended sequences such as primer dimer and unintended PCR products (sometimes referred to as non-specific PCR products). This is increasingly likely the more regions are targeted in parallel. In some embodiments the primers are designed and selected specifically to reduce the amount of primer dimer and unintended PCR products produced. In alternative embodiments primer dimer and/or select unintended PCR products are removed based on their size. This is achieved by first identifying the size of the intended PCR products (e.g. 160 bp) and then removing products that are either smaller or larger than the intended sequences (or both). In some embodiments, all DNA products 10, 15 or 20 or more bases shorter than the smallest intended product are removed as example. In such embodiments magnetic beads may be used to selectively enrich molecules above or below a certain size following PCR amplification. In alternative embodiments an automated gel electrophoresis system such as the Pippin Prep (Sage Science) or LightBench (Yourgene Health) may be used. In alternative embodiments, the PCR primers may contain cleavable bases. Following PCR the primers may be removed through cutting the cleavable bases (effectively eliminating primer dimer. Barcodes then may be added through either ligation or through end repair followed by a further round of PCR. In some embodiments more than one of these steps may be used.

In some embodiments, reagents to target the variants (e.g. capture baits or multiplex PCR primers) may be designed for all variants, then rather than selecting variants or regions, the best combination of primers or baits is selected. The primers or baits may be ranked and selected based on a combination of the score of all variants or regions targeted by each primer, pair of primers or baits and the predicted ability to amplify and/or enrich and/or sequence the targeted variants or regions within a multiplex of the other primers or baits. As would be apparent, it may be advantageous to select and rank the primers or baits in this way rather than the variants or regions. This is because the output of the assays is the integrated analysis of the collective results of multiple variants and it may therefore be preferable in some embodiments to assess larger numbers of variants at the expense of a few variants which may score highly but be challenging to multiplex with others.

In one embodiment, the best multiplex assay is designed after the top variants are selected.

In any embodiment, the patient has or had cancer or has a clonal growth that is not yet cancer but has the potential to transform. In some embodiments, the patient has undergone or is undergoing treatment for the cancer.

In any embodiment, the DNA is cell-free DNA, e.g., cell-free DNA is isolated from a fluid, such as blood plasma, blood serum, cerebrospinal fluid, urine, saliva, stool, amniotic fluid, aqueous humour, bile, breast milk, cerumen, chyle, exudates, gastric juice, lymph, mucus, pericardial fluid, peritoneal fluid, pleural fluid, pus, sebum, serous fluid, semen, sputum, synovial fluid, sweat, tears, vomit or whole blood. In a preferred embodiment the cfDNA is isolated from blood plasma. In other embodiments, the DNA may be isolated from cells, e.g., bone marrow cells, cells from a lymph node or circulating white blood cells, in the case of a blood cancer or cells from a lymph node, cells from a tumors margin or other sample types such as CSF and whole blood that are currently screened for the presence of cancer cells from solids tumors presently by other means. The cells may be obtained from a tissue sample (e.g. cancer tissue sample or suspected cancer tissue sample or tissue sample containing or suspected of containing a cancer cell) or fluid sample (e.g. any of the fluids listed above) from a patient.

The fraction of cancer DNA in the test sample of DNA may be equal or less than 0.0005%, equal or less than 0.01%, equal or less than 0.005%, equal or less than 0.002%, or equal or less than 0.001%. In some embodiments, a detectable fraction of cancer DNA in the test sample of DNA may be from about 0.0001%, however the actual LOD and LOQ may vary. In some embodiments, the whole test sample (i.e. before aliquoting) comprises less than 25,000 genome equivalents of DNA (e.g. cfDNA), e.g., less than 20,000, less than 10,000, less than 5,000, or less than 1,000 genome equivalents of DNA. In some embodiments the test sample (before aliquoting) comprises from about 100 to about 25,000 genome equivalents of DNA. In some embodiments, the test sample comprises from about 10 ng to about 100 ng of DNA. In some embodiments, the test sample comprises at least 10 ng, at least 20 ng, at least 30 ng, at least 40n, at least 50 ng, at least 60 ng, at least 70 ng, at least 80 ng, at least 90 ng, or at least 100 ng of DNA. IN some embodiments, the test sample comprises 66 ng of DNA. Genome equivalents refers to amplifiable copies.

In some embodiments, the number of aliquots and the maximum number of molecules per aliquot is adjusted based on the total number of input molecules and the estimated background error rate such that the number of input molecules in a single aliquot is low enough that if a single variant molecule were present it would produce a signal significantly different to background.

In any embodiment, for each aliquot of each sequence variation, the read depth of step (a) may be at least 10,000, at least 25,000, at least 50,000 or at least 100,000, at least 200,000 or at least 500,000. In any embodiment, for each aliquot of each sequence variation, the read depth of step (a) may be from about 10,000 to about 500,000. In any embodiment, for each aliquot of each sequence variation, the read depth of step (a) may be from about 10,000 to about 200,000. In any embodiment, the method may comprise measuring the amount of DNA in the test sample prior to step (a).

In any embodiment, the sequences of the target regions may be enriched from the test sample prior to step (a) by PCR or by hybridization to a nucleic acid probe or using a one sided PCR approach wherein there is a universal sequence on one side of the target DNA molecule and at least one and optionally a further nested primer are used to target the other side of the molecule. Other methods known to those skilled in the art such as Linked Target Capture, Molecular inversion probes and ATOM Seq may also be used.

As noted above, the present method may be done using a threshold-based approach. In these embodiments, any target region in any aliquot may be determined to contain at least one mutant molecule: i) if the estimate of the number of molecules that have the sequence variation in step b is 1 or greater, ii) if the probability calculated in step b is above a specificity threshold (e.g. 95%, 99%, 99.9%), iii) if the frequency is above the threshold, or iv) by calculating a likelihood ratio for each variant in each aliquot between the likelihood of observing the estimates in (b) in samples: (i) if cancer DNA is present and (ii) if cancer DNA is not present, then confirming whether the result is at or above a threshold. In some embodiments where a target region contains 2 variants the region may be determined to contain at least one mutant molecule if signal for both variants is present within the same sequence.

In some embodiments, cancer DNA may be determined in step (c) of the method: i) if there are equal or more than a threshold number of target regions in any aliquots that are determined to contain at least one mutant molecule, and/or ii) if there is at least 2 or at least 3 aliquots determined to contain at least one target region with at least one mutant molecule. In these embodiments, the threshold number of target regions may be: i) 2 or more (e.g., 3, 4, 5 or 10 or more) target regions in any aliquots that are determined to contain at least one mutant molecule, or ii) determined by combining the estimated rate of high signal background events for all target regions and aliquots to determine a threshold where one would expect the number of high signal background events to occur less than 5%, 0.5%, 0.1% or 0.01% or 0.001% of the time (for example, if there were 4 aliquots and 48 target regions, and for the specific combination of target regions and variants within these regions, it was estimated that you would get 4 of more high signal events across all aliquots less than 0.01% of the time, then a threshold of 4 would be set) or iii) A score rather than a fixed number of target regions or variants and wherein the threshold score is either 2 or 3, and wherein a positive target region or variant contributes a different score depending on its rate of high signal background events. In one embodiment, variants or classes of variants that never have high signal background events are given a score of 1 and the remaining variants or classes of variants are split into 1 or more groups based on their rate of high signal background events and given a lower score. For example there may be two groups. The 50% of variants or variant classes with the lowest rate of high signal events receive a score of 0.75 whilst the 50% with the highest rate get a score of 0.5 whenever positive.

In any embodiment, the threshold frequency of step (b) may be determined using a binomial, over-dispersed binomial, Beta, Normal, Exponential or Gamma probability distribution model of the background error rate for the sequence variation and wherein the frequency is selected such that a signal would be observed above this less than 5%, 2%, 1%, 0.1%, 0.01% or 0.001% of the time, depending on the desired pre-defined per variant specificity, when no mutant molecules are present.

Further details, alternative steps and embodiments of the present are described below.

Sequence Variations that are Associated with the Patient's Cancer

The present method involves analyzing multiple sequence variations that are associated with the patient's cancer in a sample, where such sequence variations are believed to be present in the cells of a patient's cancer. Any individual sequence variations may be a driver mutation or a passenger mutation and, a sequence variation may be clonal or non-clonal. The sequence variations used in the present method are cancer-associated in the sense that they are believed to be only in the cancer cells and not the non-cancerous cells in the patient. The set of mutations that define a patient's cancer are patient-specific in the sense that they vary from patient to patient, although some mutations (e.g., in KRAS, etc.). may occur in several patients and/or in several different types of cancer. Because the positions of passenger mutations in the genome are difficult to predict beforehand (although there may be some hotspots) and the positions of the sequence variations differ from patient to patient, the sequence variations that are analyzed in the present method may be identified on a patient-to-patient basis. In some embodiments, the sequence variations can be identified from samples where the cancer fraction is higher—for example, a bone marrow aspirate, a tissue biopsy sample or isolated circulating cancer cell(s). For example, the sequence variations may have been identified by sequencing DNA isolated from a bone marrow aspirate, tumor tissue biopsy or surgical resection, from circulating tumor cells (CTCs), from other cells that are no longer part of the tumor tissue but are not circulating such as those in the urine or stool samples, or cell-free DNA from the patient, where the sample from which the DNA is extracted was obtained from the patient prior to treatment for cancer when ctDNA levels are more likely to be high. In some embodiments, multiple sample types or multiple samples from different sites on the same sample, or multiple samples from the same patient originating from different sites in the patient, may be sequenced in order to determine clonality. A variant may be considered clonal when it is present in multiple such different samples, or if clonality can be inferred from sequence reads generated from bulk tumor tissue. Clonality can be difficult to determine as tumors are often heterogeneous and quantifying heterogeneity from bulk sequencing data is challenging. Various approaches have been proposed to determine clonality, including Bayesian mixture models, clustering probability distributions of cancer cell fractions, and phylogenetic methods. Software tools for determining clonality include PyClone-VI, EXPANDS, QuantumClone, and Phylo WGS. See also Gillis, S., Roth, A. PyClone-VI: scalable inference of clonal population structures using whole genome data. BMC Bioinformatics 21, 571 (2020), the contents of which are incorporated by reference in their entireties. Sequencing of multiple different or bulk samples may be done by whole genome sequencing, exome sequencing or targeted sequencing (e.g., by sequencing a panel of cancer genes or by sequencing a panel of sequences that are hotspots for mutations), etc. as described above. As would be apparent, the patient may be a cancer patient, where the patient has undergone, may be undergoing treatment for the cancer or may be about to undergo treatment. In other words, the sequence variations may be identified in a sample in which they are present at a relatively high level, e.g., in a sample that was collected before any cancer treatment has been initiated.

Depending on how the method is performed, the sequence variations may be identified before the test sample has been analyzed or at the same time as the test sample is being analyzed. As such, some embodiments of the present method may use “pre-identified” sequence variation, where “pre-identified” sequence variations are sequence variations that have previously been identified as being associated with a patient's cancer, e.g., before or during treatment. In other embodiments, the sequence variation is not pre-identified and, instead, the sequence variations may be identified by comparing sequence reads from the test sample to sequence reads obtained from control samples (e.g., positive and negative control samples, as described below). In some embodiments, sequence variations may be identified in parallel to the analysis of the test sample (i.e. without the need for “pre-identification”).

The sequence variations analyzed in the method may be independently single nucleotide variations, indels, transpositions or rearrangements. In general, the sequence variations can be identified by sequencing DNA isolated from a tissue sample (e.g., a biopsy, surgical resection or fine needle/large needle aspiration) that comprises cancer cells or sequencing cell-free DNA from the patient (e.g., whole genome sequencing, exome sequencing or a targeted sequencing approach), where multiple regions are sequenced. For example, in some embodiments a list of sequence variants may be obtained through sequencing at least 50 kb of cancer DNA, through targeted sequencing of a large region of the genome or whole genome sequencing, where the cancer DNA is obtained from either tumor tissue (e.g., a biopsy) or a sample expected to have high levels of cancer DNA in it (such as a pre-treatment plasma DNA sample). In some embodiments just cancer DNA is sequenced. In an alternative embodiment, both cancer DNA and DNA expected to be non-cancerous, such as whole blood, buffy coat, apparently non-cancerous tissue adjacent to the tumor or buccal swab may be sequenced. Variants may be classified as somatic or germ line either by assessing the cancer and non-cancerous DNA or by assessing just the cancer DNA and using the variant allele fractions in addition to optionally using other features as is known in the art.

In some cases, analysis of the initial cancer DNA sample may result in a list of candidate sequence variations, where some of the candidate sequence variations are eliminated to produce a list of pre-identified sequence variations. In some embodiments, this method may comprise obtaining a list of candidate variants that are believed to be somatic from the patient whose sample is being assessed (e.g., by sequencing a biopsy) and then prioritizing the variations, as previously described. For example, in these embodiments, the prioritization may be based on, e.g., the probability of being a real variant as opposed to a sequencing artefact, probability of being a somatic genetic abnormality, the probability of being a clonal mutation, an estimate of the error rate, an estimate of the compatibility to multiplex with other variants and/or the map-ability of the variant and surrounding regions, the estimated number of copies of the variant in each cancer such as presence in a region of copy number gain or an amplification, in episomes or double minute chromosomes or regions of chromoplexy etc. In addition to prioritizing the candidate variations, one or more of the candidate sequence variations may be eliminated and only a subset of the candidate sequence variations may be selected for future analysis. For example, after the candidate sequence variations are identified, the target regions that contain those sequence variations may be sequenced in DNA from non-cancerous cells (buffy coat, white blood cells, buccal swab, or adjacent tissue). This sequencing may be performed using that same approach as used for sequencing the cancer DNA or the sequencing may be performed using an assay designed to detect variants identified in the cancer DNA. Any variants identified in these non-cancerous cells may be eliminated from the candidates as being likely to be germline polymorphisms or clonal hematopoiesis and the remainder of the sequence variations can be prioritized. For example, in some embodiments, the method may further comprise sequencing at least some of the target regions in the DNA of white blood cells from the patient. In these embodiments, the method may involve comparing the candidate genetic variations to the genetic variations called using the white blood cell DNA. If a variation is identified in both samples, then it may be eliminated from being a pre-identified sequence variation. This embodiment provides a way to identify variations that may be potentially due to clonal hematopoiesis of indeterminate potential (CHIP) (see, generally, Funari et al, Blood 2016 128:3176 and Heuser et al, Dtsch. Arztebl. Int. 2016 113:317-322, the contents of which are hereby incorporated by reference in their entirety) and germ line variants so that they can be eliminated from future analysis. In an alternative embodiment, the method may involve comparing the candidate genetic variations to the genetic variations called using the apparently normal tissue adjacent to the tumor. If a variation is identified in both samples, then it may be eliminated from being a pre-identified sequence variation. This embodiment provides a way to identify variations that may be potentially due to cancer field effect and germ line variants so that they can be eliminated from future analysis

As such, in any embodiment, the method may comprise sequencing one or more positive and/or negative controls samples (which may be run prior to or at the same time as the test sample). As would be apparent, this assay is “personalized” in that the initial cancer DNA sample, the control samples and the test sample are obtained from the same individual. Positive and negative controls samples include but are not limited to: cancer DNA from biopsy or surgery sample either from the primary tumor or a metastasis, buffy coat DNA, buccal swab DNA, whole blood DNA, DNA isolated from non-cancerous tissue (e.g., adjacent tissue) or reference DNA. In these embodiments, sequence variations that are not detected in the cancer DNA may be excluded and wherein sequence variations that are detected in the buffy coat, buccal swab, adjacent non-cancerous or whole blood are excluded. In any embodiment, a sequence variation may be prioritized based on one or more factors which may include: clonality, mappability, estimated error rate, distance from another selected variant, compatibility with other variants when designing a multiplex PCR or hybrid capture panel, predicted ability to sequence, presence within a region of copy number gain or amplification, and proximity of any germ line variants either in cis or trans which may be used for enriching the mutant allele. Methods that would enable enrichment of sequence variations in close proximity to a germ line variant include performing allele specific PCR wherein at least one of the primers is specific to the strand with the germline change and the variant is on the same stand (in cis), or targeting the germ line change for example with restriction enzyme, cas9 or similar method when the variant is on the opposite strand (or in trans) in order to remove wild type strands. In other embodiments a sequence variation may be prioritized based on its suitability for variant enrichment methods such as allele specific PCR, COLD-PCR or other methods know to those skilled in the art.

As may be apparent, the sequence variations analyzed in the method may vary from patient to patient such that the sequence variations analyzed in the method are “customized” to each patient. As such, in many embodiments, the method may comprise identifying a first set of sequence variations from a DNA sample from a first patient, a second set of sequence variations from a DNA sample from a second patient, a third set of sequence variations from a DNA sample from a third patient, and so on.

### Aliquot-Based Measuring

The aliquot based-measuring method may be practiced in a variety of different ways. In some embodiments, target regions that have the sequence variations may be sequenced using an “amplicon-based” approach in which the target fragments that have pre-identified sequence variations are directly amplified by PCR from the sample. In some embodiments the test sample may first be pre-amplified, for example by whole genome amplification. Pre-amplification may be achieved, for example, by the ligation of adaptors and performing PCR targeting the ligated adaptors. In these embodiments, the sequencing adapters may be added during amplification or may be ligated on after the amplification. In other embodiments, target regions that have pre-identified sequence variations may be sequenced using an “target enrichment-based” approach in which adapters are ligated to the sample, and fragments containing the target regions are enriched by hybridization to a nucleic acid probe prior to amplification using primers that hybridize to the adapters. In such embodiments, either aliquot ligation reactions may be performed, or adaptors with a plurality of barcodes may be ligated onto the DNA enabling the effective separation of groups of molecules into separate barcode groups or “aliquots”. As such, sequences of the target regions can be enriched from the sample by PCR or by hybridization to a nucleic acid probe. Other enrichments methods may be used. In other embodiments any other method with either physical replication or use of molecular barcodes may be utilized such as Molecule Inversion Probes (MIP) or Anchored Multiplex PCR (AMP). Some of the principles of the amplicon-based method are described below. Similar concepts can be applied to the target enrichment approach. In some embodiments the variant sequences may be enriched during the targeting step using methods including COLD-PCR, allele specific PCR targeting the variant, allele specific PCR targeting an adjacent germline change, digestion of wild type sequence through the utilization of adjacent germline changes or other methods known to those skilled in the art.

In embodiments that employ pre-identified sequence variations, multiple primer pairs are obtained after the pre-identified sequence variations have been identified, where each primer pair amplifies a target region that has one or more of the pre-identified sequence variations. In some embodiments, the length of each amplicon, independently, may be in the range of 50 bp to 500 bp, e.g., 70-150 bp, although longer or shorter amplicons may be used in some implementations. In some embodiments some of the variants are rearrangements. In these embodiments, primers are designed with one primer 3′ of the rearrangement and one primer 5′ wherein the rearranged sequence is used to design the primer pairs and the primers are specifically deigned to amplify the rearranged sequence. After the primer pairs have been obtained, the method may comprise setting up at least two multiplex PCR reactions (e.g., up to 10 multiplex PCR reactions, such as 2, 3, 4, 5, 6, 7, 8, 9 or 10 multiplex PCR reactions) each containing a portion of the same sample (i.e., different aliquots of the same sample). In this step, the multiplex PCR reactions can be identical to one another in that all the reactions have the same primers and different portions of the same sample. In this method, the number of aliquots and the maximum number of molecules per aliquot may be adjusted based on the total number of input molecules and the estimated background error rate such that the number of input molecules in a single aliquot is low enough that if a single variant molecule were present it would produce a signal significantly different to background. As would be apparent, each multiplex PCR reaction should contain compatible primers, where compatible primers are designed to specifically amplify regions of interest producing amplicons that correspond to the PCR primer pairs while minimizing the production of primer dimers and unintended or non-specific PCR products, when the reaction is subjected to appropriate thermocycling conditions with an appropriate template for the primers. Typically, although not always, each primer pair amplifies a single region of interest in a multiplex PCR reaction. Conditions for performing multiplex PCR and programs for designing compatible primers are well known (see, e.g., Sint et al, Methods Ecol Evol. 2012 3:898-90 and Shen et al BMC Bioinformatics 2010 11:143, the contents of which are each hereby incorporated by reference in their entireties). Compatible primer pairs may be designed using any one of a number of different programs specifically designed to design primer pairs for multiplex PCR methods. For example, the primer pairs may be designed using the methods of Yamada et al. (Nucleic Acids Res. 2006 34: W665-9), Lee et al. (Appl. Bioinformatics 2006 5:99-109), Vallone et al. (Biotechniques. 2004 37:226-31), Rachlin et al. BMC Genomics. 2005 6:102 or Gorelenkov et al. (Biotechniques. 2001 31:1326-30), the contents of which are each hereby incorporated by reference in their entireties. In some embodiments, the method may employ at least 5 pairs of compatible primers, e.g., at least 10, at least 50, at least 100, at least 1000 or at least 5000 pairs of compatible primers. The amplicons amplified can be of any suitable length and may vary in length. In some embodiments, sequence variations may be prioritized based on the likely compatibility of primer designs in a multiplex PCR.

Next, the amplicons produced by thermocycling the reaction, or amplification products thereof (if the amplicons are re-amplified by universal primers that hybridize to 5′ tails in the primers, for example) are sequenced to produce sequence reads. The various aliquot PCR reactions should produce replicate amplicons, where “replicate” amplicons are amplicons that are amplified by the same primers in the aliquots. Replicate amplicons generally have the same sequence (except for PCR errors, variations corresponding to genetic variations in the sample, any variations in the PCR primers, etc.).

In sequencing the amplicons, the amplicons derived from each different multiplex PCR reaction may be sequenced separately to one another or the amplicons may be barcoded with an aliquot identifier and then pooled prior to sequencing. In some embodiments, the primers in the multiplex PCR reactions may have a 5′ tail that contains the aliquot identifier such that, after the PCR reactions have been completed, the sequence of the 5′ tail of the primers is present in the amplicons. In other embodiments, the multiplex PCR reactions can be done without using primers that have a 5′ tail that contains an aliquot identifier. In these embodiments, the PCR products may be barcoded with an aliquot identifier in a second round of amplification that uses PCR primers that have a 5′ tail containing an aliquot identifier. Adapter sequences could also be ligated onto the products. Either way, the amplicons may be amplified prior to sequencing, using primers that have a 5′ tail that provides compatibility with a particular sequencing platform. In certain embodiments, in addition to an aliquot identifier, one or more of the primers used in this step may additionally contain a sample identifier. In some embodiments, one or both of the primers may contain a barcode, which either independently or in combination may be used to identify both the sample and aliquot. If the primers have a sample identifier, then products derived from different samples can be pooled prior to sequencing. In some embodiments, the target specific primers contain from 5′ to 3′ a universal “tagging” sequence, an optional aliquot barcode sequence followed by a sequence designed to the target of interest. The primers used to further amplify the initial products may additionally or alternatively contain a 5′ tail (e.g. a sequencing adaptor) that provides compatibility with a particular sequencing platform, a sample barcode and optionally a aliquot barcode or a barcode that identifies both the sample and aliquot, and a sequence that can bind to either part or all of the reverse complement of the tagging sequence present on the target specific primers. Typically, the forward and reverse primers will have different tagging sequences. As would be apparent, the primers used for the amplification step may be compatible with use in any next generation sequencing platform in which primer extension is used, e.g., Illumina®'s reversible terminator method, Roche's pyrosequencing method (454), Life Technologies' sequencing by ligation (the SOLID platform), Life Technologies' Ion Torrent platform or Pacific Biosciences' fluorescent base-cleavage method and any other platforms e.g. Oxford Nanopore. Examples of such methods are described in the following references: Margulies et al (Nature 2005 437:376-80); Ronaghi et al (Analytical Biochemistry 1996 242:84-9); Shendure (Science 2005 309:1728); Imelfort et al (Brief Bioinform. 2009 10:609-18); Fox et al (Methods Mol Biol. 2009; 553:79-108); Appleby et al (Methods Mol Biol. 2009; 513:19-39) English (PLOS One. 2012 7: e47768) and Morozova (Genomics. 2008 92:255-64), which are all hereby incorporated by reference in their entirety for the general descriptions of the methods and the particular steps of the methods, including all starting products, reagents, and final products for each of the steps.

In alternative embodiments, the aliquot-based sequencing could target a panel of mutation hotspots, a panel of cancer genes. Alternatively, the sequencing step could be performed by exome or whole genome sequencing, or by sequencing at least 1, at least 5 or at least 10 MB of the genome to a suitable depth. In these embodiments, the sequence variations do not need to be “pre-identified”. Rather, the sequence variations can be identified in the same assay in which the test sample is sequenced, i.e., by comparison of the data to controls that are also run in the same assay (e.g., the same sequencing run). Once the sequence variations have been identified using the control samples, those sequence variations can be analyzed in the test sample.

The sequencing step may be done using any convenient next generation sequencing method and may result in at least 100,000, at least 500,000, at least 1M at least 10M at least 100M, at least 1B or at least 10B sequence reads per reaction. In some cases, the reads may be paired-end reads.

In embodiments that measure a level of fluorescence, a two-step PCR amplification process may be used. In the first step, the target region containing the sequence variation is first amplified, and then the amplified product is detected using a synthetic oligonucleotide (e.g., a TaqMan probe or SYBR Green) that is complementary to a specific target region. In some embodiments the primers are designed to only amplify the target region if the variant is present. This may be achieved by designing one of the primers to overlap the variant of interest. The probe consists of a “reporter” dye, which fluoresces when excited at a specific wavelength, and a “quencher” dye, which absorbs the fluorescence emitted by the reporter dye. The probe may be designed so that the reporter and quencher dyes are in close proximity to each other when the probe is not bound to the target region. When the probe is bound to the target region, the reporter and quencher dyes are separated through exonuclease activity of a polymerase, allowing the reporter dye to fluoresce. Alternatively the probe may not be required to bind to the target region but instead becomes separated when a mediator sequence is released during the initial PCR and binds to the probe, as described in US Patent Publication 2014/0315747, the contents of which are hereby incorporated by reference. The fluorescence emitted by the probe is measured and used to quantify the amount of the target region containing the sequence variation present in the sample.

### Processing Measurements, Estimating Variant Molecules, and Determining Presence of Cancer DNA

The measurements (e.g., sequence reads) are then processed computationally. The initial processing steps may include identification of barcodes (including sample identifiers or aliquot identifier sequences) and trimming reads to remove low quality or adaptor sequences. Trimming of reads can be achieved, for example, by inputting the sequence file into one of the available automated trimming scripts, for example Trim Galore! (developed by The Babraham Institute). In addition, quality assessment metrics can be run to ensure that the dataset is of an acceptable quality. For example, per-base quality scores may be used to determine whether certain positions within a sequence read (such as that of a variant) are trustworthy.

After the sequence reads have undergone initial processing, they may be analyzed to identify which reads correspond to the target regions. These sequences can be identified because they are identical or near identical to the sequence of a target regions. As would be recognized, the sequence reads that are identical or near identical to the target region can be analyzed to determine if there is a potential variation in the target sequence. Sequences may be aligned with a reference sequence, e.g., a genomic sequence, in this method or matched to a database of expected sequences.

After the sequence reads have been processed, the method may comprise, for each aliquot and each sequence variation, counting the number of sequence reads that have the sequence variation and counting the total number of sequence reads. Methods for counting reads may be adapted from those described by e.g., Forshew et al (Sci. Transl. Med. 2012 4: 136ra68), Gale et al (PLOS One 2018 13: e0194630), and Weaver et al (Nat. Genet. 2014 46:837-843), all hereby incorporated by reference in their entirety. Similar results can be obtained using an approach that employs molecular indexes. In these methods the total number of molecules sequenced and the number of variant molecules can be estimated using the indexes. Such molecule identifier sequences may be used in conjunction with other features of the fragments (e.g., the end sequences of the fragments, which define the breakpoints) to distinguish between the fragments. Molecule identifier sequences are described in (Casbon Nucl. Acids Res. 2011, 22 e81), hereby incorporated by reference in its entirety.

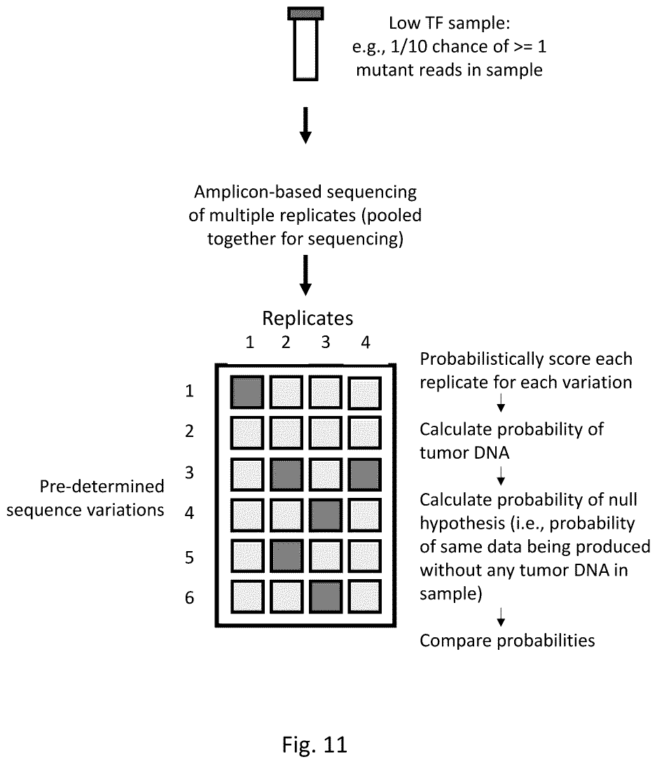

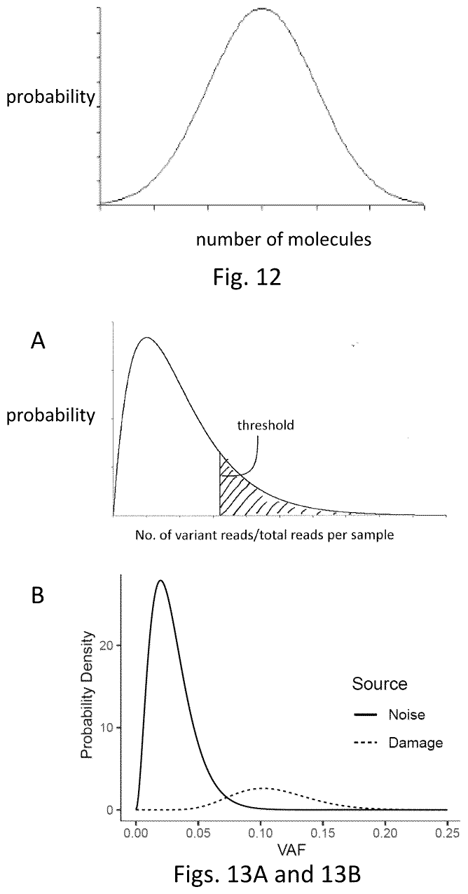

As illustrated in FIG. 11, after counting the number of sequence reads that have the variation and counting the total number of sequence reads (or otherwise determining an amount of signal), an estimate of the number of molecules in the original sample before amplification, that had the sequence variation can be determined for each aliquot of each target region. Alternatively, one can calculate the probability that there is at least one molecule that has the sequence variation, for each aliquot of each target region. The latter can be derived by, for example, summing the individual probabilities for all non-zero numbers (i.e., all positive integers) of counts of possible variant molecules up to the total number of input molecules. In these embodiments, the estimate can be a probabilistic estimate, meaning that the estimate is not a point estimate but is a probability distribution. This step may be done by assigning each possible number of variant molecules in the aliquot with a probability, which may be done via a probability density function, an example of which is illustrated in FIG. 12. In these embodiments, for each aliquot and target region the estimate of the number of molecules that have sequence variation or the probability that there is at least one molecule that has the sequence variation may be calculated using: (i) the number of sequence reads that have the sequence variation, (ii) the total number of sequence reads, (iii) the number of molecules input into each aliquot, and (iv) the estimated background error rate for the sequence variation. In these embodiments, the sequence of the target region will be represented by a number of sequence reads (e.g., at least 10,000 reads, although this number can vary depending on the number of aliquots that are sequenced) and some of those reads may contain the sequence variations. These reads can be counted in order to provide input values (i) and (ii). Input value (iii) can be calculated by measuring the amount of DNA in the DNA sample prior to initiating the method. This can be done, for example, by measuring the total amount of DNA, the total amount of double stranded DNA, the total amount of double and single stranded DNA, the total amount of DNA within a specific size range or the total amount of DNA that can be amplified using primers with specific parameters such as amplicon size. This step can be done by digital PCR, qPCR, fluorometrically, through electrophoresis or using any of a variety of kits or other strategies. The estimated background error rate for each sequence variation, i.e., input value (iv), can be determined from prior sequencing reactions, e.g., sequencing reactions done on samples that are known to not have the sequence variation or on samples from individuals not known to have cancer and therefore not anticipated to have large numbers of somatic variants. Specifically, background error rate for each variation can be estimated through the sequencing of similar variants in DNA not expected to contain somatic mutations in the similar variants being assessed either in the same run, in historical runs or using historical runs then adjusting using select control bases (or bases not known to contain variants), and wherein variants are considered to be similar based on features which may include; the base change, the type of base change (transition/transversion) and the trinucleotide context, the pentanucleotide context, the position in the amplicon in reference to a primer, size of insertion, type and number of inserted bases, size of deletion, type and number of deleted bases or class of rearrangement, for example tandem duplication. A hypothetical error model is shown as a frequency distribution in FIG. 13A or a mixture model shown in FIG. 13B. In these examples, multiple samples (e.g., several hundred samples) that are not known to contain somatic variants are sequenced, and the fraction of sequence reads that have a particular type of sequence variation can be calculated for each sample. The variant sequence reads are largely caused by errors that occur during PCR, base mis-calls and pre-PCR events such as DNA damage (e.g., the oxidation of guanine to 8-oxoguanine, which base pairs with A, resulting in what appears to be a G to T variation in a sequence read). These fractions can be plotted as a frequency distribution which, in turn, can be used to calculate the probability of whether a sequence variation observed in a sequence read is really a genetic variation.

The presence or absence of cancer DNA in the sample can then be determined using the estimates (or probabilities) of variant molecules in each target region from each aliquot of the original sample. In some cases, the data can also be used to estimate the overall cancer DNA fraction in the sample. This estimate may be the most likely amount of cancer DNA or a range of likely amounts of cancer DNA in the test sample, and may be estimated based on the fraction of variant reads or estimates of variant molecules in the original sample, such as by mean or median variant allele fraction, maximum likelihood or Bayesian posterior.

In one embodiment, the presence or absence of cancer DNA in the sample can be determined via a likelihood ratio, by comparing the likelihood of observing the results given that cancer DNA is present with the likelihood that the same results could have been generated by a sample that does not contain any cancer DNA. The value of this threshold may be determined by experiment and selected based on a desired level of specificity, e.g., the threshold is selected such that a likelihood ratio would be observed above the threshold value less than 5%, 2%, 1%, 0.1%, 0.01%, or 0.001% of the time when no cancer DNA is present. If there is a higher likelihood that the same data could be produced by a sample that does not contain any cancer DNA, then the sample may not contain any cancer DNA. The first likelihood (the likelihood with cancer DNA present) may be calculated using (i) the estimated numbers of molecules with the sequence variation or probabilities, as calculated above for each aliquot of each target region; and, optionally, (ii) the cancer DNA fraction estimated in the sample. The second likelihood (the likelihood for the null hypothesis) may be calculated using (i) the probabilistic estimates or probabilities, as calculated above; and (ii) the estimated rate of high signal background events, where a “high signal background event” is an event which is not accounted for by the simple model of the background error rate per read. After the likelihood of there being cancer DNA in the sample and the likelihood of the null hypothesis have been calculated, they can be compared to obtain a likelihood ratio and, in turn, the likelihood ratio can be compared to a threshold. In some embodiments a likelihood ratio is determined for each aliquot of each target region. The individual likelihood ratios are then combined into a cumulative likelihood ratio score across all the regions and aliquots of the sample. A likelihood ratio that is at or above the threshold indicates that the DNA sample contains cancer DNA. Alternatively, the likelihood ratio can be interpreted as a probability that the sample contains cancer DNA, either directly or by comparison to a reference distribution calculated on control samples.

Specifically, as noted above there are at least three types of errors in the model in FIGS. 13A and B: errors that occur during PCR, base mis-calls during sequencing and pre-PCR events such as DNA damage. The pre-PCR errors are “high signal” in the sense that they are rare (they are not associated with every sample) but when they do occur, they result in a much higher fraction of variant reads than the other errors consistent with variant molecules being present in the original sample, i.e. they mimic the appearance of a true positive ctDNA variant. In some instances, errors that occur in the first one, two or three cycles of PCR may also produce high signal events. The rate of such errors can be determined using a variety of different methods. In some cases, an error distribution or distribution of error probability may be used. In these embodiments, the errors skew the distribution as illustrated in FIGS. 13A and B. Analysis of such an error distribution allows the high signal events to be identified as separate events. For example, in some cases, the events can be identified using a threshold (e.g., an event that is one, two or three standard deviations from the mean or median) as illustrated in FIG. 13A. Such a threshold can change from variation-to-variation but, in general, they can be identified as having a frequency that is above a defined threshold as illustrated in FIG. 13A. These high signal events can be separately modeled and used to determine the rate of high signal background events for each sequence variation.

In another embodiment, a determination of whether the test sample contains cancer DNA is calculated by using a mixture model (FIG. 13B) incorporating: (i) the estimates or probabilities of variant molecules in each aliquot of each target region, the estimated rate of high signal background events and optionally a prior estimate of the cancer DNA fraction in the test sample. The mixture model can be used to calculate a likelihood ratio between the likelihood of observing the estimated rates if (i) cancer DNA is present (ii) if cancer DNA is not present. The likelihood ratio can be compared to a threshold, wherein an output that is at or above a threshold indicates that the test sample contains cancer DNA. Such a threshold for either method may be determined by analyzing a plurality of samples not known to contain cancer DNA and determining a distribution of results then setting a thresholds such that a false positive would be expected less than 0.01% of the time, less than 0.1% of the time, less than 0.5% of the time, less than 1% of the time or less than 5% of the time.

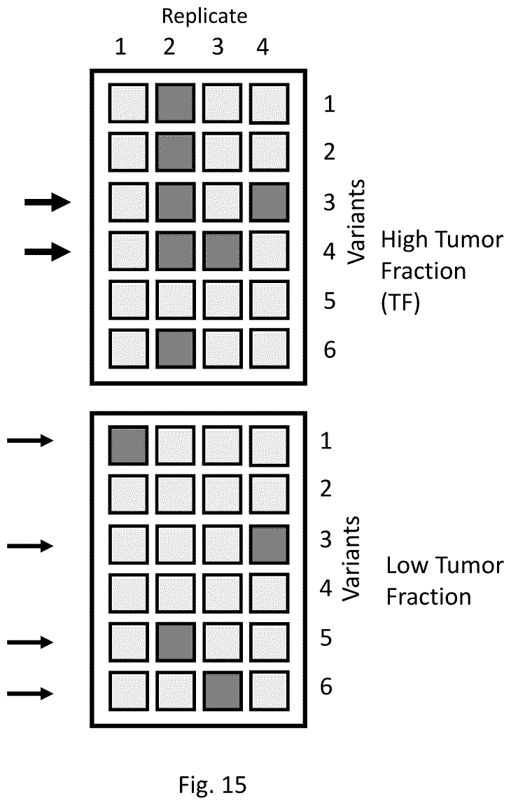

In some embodiments, the probabilistic estimates or probabilities for sequence variations that are identified in a statistically improbable number of the aliquots based on the estimated cancer DNA fraction are excluded, prior to calculating likelihood of there being cancer DNA in the sample, or prior to determining if sufficient target regions, variants and or aliquots are above a threshold to indicate cancer DNA is present. For example, if the estimates or probabilities for most aliquots of most variations are relatively low indicating that they are unlikely to contain variant DNA, except for occasional aliquots that are relatively high, it would be statistically improbable that one sequence variation would be present in all or almost all aliquots with a relatively high probability. As a further example, in an embodiment with 4 aliquots, if the evidence for most variants supports either 0 or 1 aliquots containing variant DNA, any variants where the evidence for all 4 aliquots supports the presence of variant DNA is likely to be an outlier. These outliers (which may be caused by “noisy bases”, or non-cancer specific changes that are derived from CHIP, for example) can be identified and eliminated from the calculation. In another example, using the number of test DNA molecules added to each aliquot and an estimate of the tumor fraction calculated using all variants (or a subset), the chance of each individual variant in each aliquot containing at least one cancer molecule can be calculated. The number of aliquots above a threshold can then be compared with the total number of aliquots to determine if the variant is giving an improbable result. In some embodiments the copy number of each variant is corrected for during this calculation. This concept is illustrated in FIG. 14.

In the present method, variant-containing regions that result in more aliquots than would be expected with a high signal (given the cfDNA concentration and the estimated ctDNA fraction) can be identified and eliminated. This may be calculated using the probability of sampling at least one ctDNA molecule per partition given a known cfDNA concentration and an estimated ctDNA fraction. Variants for which this is statistically improbable (e.g., p<0.05) may be excluded. For example, if each of 4 partitions had a 0.2 chance of containing a variant (based on the estimated ctDNA fraction and number of input molecules), the likelihood of seeing 2 partitions with a high score can be calculated.

For clarity, some embodiments of this method does not involve identifying (“or calling”) variations in the different aliquots. Specifically, some embodiments of the method does not involve determining whether the frequency of a potential sequence variation is above or below the threshold in each aliquot. Rather, these embodiments of rely on analysis of the data as a whole.

While the method can be practiced on any type of sample that has cancer DNA in it, the method finds most use for the analysis of limited samples in which the fraction of cancer DNA is less than 0.01% (i.e., is less than 100 ppm), since this is when samples that contain cancer DNA become indistinguishable from samples that do not contain cancer DNA in other assays. For example, in some embodiments, the method may be used to detect cancer DNA in samples that contain from about 0.0001% (1 ppm) (for example from about 0.0001% (1 ppm) to about 1% (10000 ppm)) cancer DNA, optionally where the sample (prior to aliquoting) comprises less than 25,000 genome equivalents of DNA (e.g., 100 to 10,000, 500 to 5000 or 2000 to 20,000 genome equivalents of DNA), although these numbers may vary. Moreover, in order to obtain statistically significant results, each aliquot of each target region can be sequenced to a read depth of at least 5,000, at least 10,000, at least 20,000 or at least 100,000, as desired.

### Estimating the Amount of Cancer DNA

In some embodiments, the amount of cancer DNA may be measured as a total number of variant containing molecules. In another embodiment, the amount of cancer DNA may be measured as an estimated variant allele fraction (VAF). In some embodiments, a mean or median VAF may be generated (i.e. a mean or median of all the variants analyzed), in other embodiments a corrected mean or median VAF may be determined (i.e. the mean or median level across the variants after subtracting a previously pre-determined offset or baseline error rate for each variant). In some embodiments, the VAF and the total number of cfDNA molecules added to the sequencing reaction may be multiplied together as a method for estimating the total number of variant tumor molecules that were added to the sequencing reaction.

In other embodiments, information obtained through sequencing the tumor tissue may be used to estimate the number of copies of each variant within a single cancer cell and this information may be used in combination with the variants detected in the sample and their frequencies to determine the number of tumor cells it represents, i.e., the “cancer cells represented”.

In some embodiments, the measure of variant containing molecules, or estimated numbers of cancer cells may be combined with the number of millilitres of fluid such as blood plasma from which the DNA was extracted in order to estimate the number of molecules per ml of sample. In examples of such an analysis one may calculate a range of outputs such as, mean variant molecules per ml of plasma, median variant molecules per ml of plasma, median tumor cells per ml of plasma or Median variant molecules per ml of CSF.

In some embodiments, this calculation may contain steps to correct for DNA lost between blood collection and sequencing analysis. This could include correcting for cfDNA extraction efficiency or correcting for library preparation efficiency. As an example, when working out the mean or median variant molecules per ml of blood plasma, one would first determine the number of mutant (i.e. variant) molecules that could be detected in the sample, and from what volume of plasma, the cfDNA sample used was extracted from. This number would then be corrected for the known number of molecules typically recovered by the extraction chemistry used and/or the rate of converting then sequencing such molecules during sequencing library preparation and analysis. Similarly, one could quantify the amount of cfDNA in the sample (ng/mL), and multiply it by the number of haploid genomes (303 per ng) and the mean variant allele fraction for any positive or all target regions. See also Example 8 herein for an example. In some embodiments, at least one synthetic spike DNA sequence with a known sequence is added to the sample prior to extraction and this sequence is analysed during sequencing to determine the efficiency of extraction and library preparation and then applied to correct previously described mutant molecules estimates (as described in WO2020174406, which is hereby incorporated by reference in its entirety). In certain embodiments, the spike sequence could contain a molecular barcode to enable counting the number of molecules successfully read.

### Estimating Limit of Detection and Limit of Quantification

As would be apparent to someone skilled in the art, a number of factors impact the sensitivity of a method such as this. Depending on the approach these factors could include the amount of DNA from the test sample added to the library preparation reaction and sequenced, the number of aliquots, the number of target regions and variants, the background error rate and the rate of high signal background events for each variant.

In some embodiments, a limit of detection and/or a limit of quantification may be determined each time a sample is analysed. In some embodiments, the amount of DNA from the sample added to the sequencing reaction is multiplied by the number of target regions in order to determine the number of DNA molecules assessed for variants. During analytical validation studies a range of samples with different numbers of molecules assessed for variants are tested in order to determine their limit of detection and/or quantification empirically. In some settings additionally the variants are separated into classes and the impact of each class is determined. When a sample is tested, its limit of detection and/or quantification is then estimated based on at least one of the number of variants, the amount of DNA added to each aliquot, the number of molecules assessed for variants and/or the class of variants assessed.

### Utilizing Cancer Signatures

It is known in the art that a range of mutational processes drive somatic mutation formation in cancer genomes and that each of these generates a characteristic mutational signature (Alexandrov, Nature 2020 578:94-101, which is hereby incorporated by reference in its entirety). Whilst some of these processes and therefore their signatures are common to many cancers others are specific to certain cancers. By sequencing a sufficiently large region of the genome such as the exome or whole genome, it is possible to detect these signatures in cancer DNA. In one embodiment of the present method, when cancer DNA from the patient is sequenced it may be analyzed in order to determine the signature(s) present. When the tumor is of unknown primary, the signature(s) may be used to infer the origin of the cancer. As example, an SBS7a signature (Alexandrov, supra) present within the tumor would be consistent with the primary tumor being a melanoma.

In another embodiment, the signature may be used to determine the likelihood that a variant identified in the tumor is a somatic change specific to the cancer rather than either artefact, germline, or CHIP. In such an embodiment a plurality of potential tumor specific somatic variants are identified by sequencing cancer DNA. The type of tumor (e.g. melanoma) is identified as are the common signatures present in that tumor type (e.g. SBS7a which are mainly C>T at TCN). Variants that are consistent with the common signatures of the cancer type are included, prioritized or given a score indicating they are more likely to be real somatic changes when selecting, ranking or scoring variants for targeted sequencing, whilst variants that are not consistent with the main signatures are either filtered out or given lower priority or score.

Method for Assessing cfDNA Quality

The method wherein the test sample is cell free DNA and prior to sequencing the cell free DNA from blood plasma, the cell free DNA is assessed to determine the quantity or proportion that is high molecular weight. Cell free DNA is typically short (~160 bp). This is because much of it is released by apoptosis of cells in the body (including cancer cells). During this process DNA is typically cut on either side of the nucleosome leaving fragments of DNA that are ~160 bp in length (and some additional fragments that are multiples of ~160 bp). When blood samples drawn into blood collection tubes are poorly handled or shipped, white blood cells may lyse and, when they do, they can release high molecular weight DNA (long DNA molecules often 1,000s of bases long) which can mask the cfDNA. As an example, if a collected blood sample is allowed to get too warm or cold (e.g., deviates outside of a range of ~4-37 C) or is kept for too long before processing to plasma (e.g., more than 10-14 days at room temperature), white blood cells can become damaged and release high molecular weight DNA. This high molecular weight DNA can result in false negatives (e.g. failure to detect actionable changes or MRD) or it can result in apparent reductions in ctDNA levels (indicating a patient is responding to therapy for example) when in reality the ctDNA is either stable or increasing. Therefore a high proportion of long DNA molecules can signify a poor sample with risk of false negative. The method wherein a ratio between the number of short DNA molecules and the number of long DNA molecules is determined and wherein short may be less than 50 bp, 60 bp, 70 bp, 80 bp, 90 bp, 100 bp, 110 bp, 120 bp, 130 bp, 140 bp, 150 bp or 160 bp and long is more than 320 bp, 480 bp, 1000 bp or 2000 bp. The method wherein if more than 1:10, 1:5, 1:4, 1:3 or 1:2 of the DNA is long the sample is flagged for potentially containing high levels of long DNA molecules that may be a sign of white blood cell DNA released after blood collection. In some embodiments, the number of short DNA molecules and number of long DNA molecules are measured using electrophoresis such as agarose gel analysis or commercial systems such as the fragment analyser or tapestation.

In one embodiment, the method of assessing cfDNA quality in a test sample is performed using PCR based approaches. Examples include using digital PCR or qPCR with primers and probes targeting both long and short regions of the genome. Either one long and one short region could be targeted or the assay could be multiplexed with a range of different sizes or multiple markers of one size and multiple markers of another size. Advantages of such a method include the ability to compensate when some regions of the genome are impacted by copy number changes. Alternatively the assays could target repetitive sequences wherein a short region of a repetitive sequence is targeted and a long region of a repetitive sequence is targeted. An advantage of such an embodiment is that less of the test DNA is required in order to measure the ratio. In another embodiment, two or more pairs of primers which target short regions of the genome are used wherein the two regions are on the same chromosome but separated by greater than 320 bp, greater than 480 bp, greater than 1000 bp or greater than 2000 bp. Replicate PCR reactions are performed on test DNA diluted such that there is typically less than a single copy of the genome per reaction in order to determine the number of times both regions amplify in the same reaction, the number of times just one or neither region amplifies in a reaction and the number of times neither region amplifies. The frequency of these three events can be used to estimate the number of long and short molecules.

In another embodiment, a method of assessing cfDNA quality in a test sample, comprises selecting at least two regions of the genome, wherein the at least two regions are separated by a distance. In some embodiments, the distance is greater than 320 bp, greater than 480 bp, greater than 1000 bp, or greater than 2000 bp. The method further comprises determining whether the at least two regions of the genome are present within the test sample. In some embodiments, this can be performed using a digital PCR or qPCR assay with primers and probes targeting the at least two regions. If signal is observed for only one or neither of the short regions, then the test sample cannot contain both of the two regions and therefore the length of the cfDNA is predominantly less than the length of any long DNA molecules, indicating the sample has been properly handled. However, if signal is observed for both of the short regions, then either there is at least one long DNA molecule containing each of the at least two regions is present in the sample, or there are two separate DNA molecules each containing one of the at least two regions. The likelihood of the latter can be determined by estimating the probability of there being two separate DNA molecules each containing one of the at least two regions, e.g. by calculating the number of expected events using (e.g.) the Poisson distribution and the degree of signal seen for each region (which may be combined over multiple assays). This can be used to estimate the quantity of DNA molecules that are short and the number that are long. If the quantity of DNA molecules in the test sample that are long exceeds a threshold (e.g., >5%, >10%, >20%) then the test sample may be flagged as potentially contaminated.

In another embodiment, next generation sequencing may be used. In one embodiment, a standard library is generated from the cfDNA by ligating on sequencer adaptor's and optionally amplifying the DNA. In an alternative example, one or more primers that target one or more repetitive regions is used to amplify the cfDNA before sequencing. Sequencing reads are then aligned to the genome and the size of the molecules determined by identifying the start and end of each sequencing read. The ratio between short and long molecules can then be obtained by grouping the sequencing reads into groups based on the length of the sequencing read then determining a ratio. In such settings it may be important to use a correction factor as PCR and next generation sequencing methods both typically have a bias for shorter DNA molecules. Alternative methods that ligate adaptors on at least one side of the cfDNA molecules and PCR using one or more targeted primers and also primers targeting the adaptors followed by NGS can be used to obtain a measure of the cfDNA lengths. In some embodiments the test sample is cell free DNA and prior to generating a sequencing library, size selection is used to enrich for shorter cfDNA molecules and increase the fraction of ctDNA wherein this enrichment may be performed using beads or size selection on a gel and wherein short molecules are those that are less than 160 bp or 150 bp or 140 bp in length.

### Utility

If the DNA sample from the patient contains cancer DNA then the patient may have cancer associated cells resulting from minimal residual disease, early relapse or metastasis, for example. ctDNA is an especially powerful biomarker in this setting because it has a half-life of approximately 1 hour so if a tumor has been fully removed any remaining ctDNA should have been cleared rapidly.

In some cases, when testing for minimal residual disease using cell-free DNA taken from a patient after treatment, it may be valuable to first confirm if the tumor releases ctDNA at a sufficiently high level for accurate minimal residual disease detection. In one embodiment a cell free DNA sample is taken prior to treatment with curative intent and tested and any patient without detectable ctDNA prior to treatment or where the probability of the sample containing cancer DNA prior to treatment is below a certain threshold may be excluded from further analysis as they release too little ctDNA for accurate minimal residual disease detection. In an alternative embodiment patients may be excluded from further analysis if the pre treatment ctDNA is estimated to be below a threshold such as 0.01% VAF, 0.005% VAF or 0.001% VAF. In another embodiment, the level of ctDNA prior to treatment is correlated with tumor volume prior to treatment as assessed by imaging in order to give an estimate of the amount of ctDNA released by a set volume of tumor and thus a standardised measure of tumor ctDNA release. Patients may be excluded for whom this standardised measure is below a set threshold for example wherein a tumor of 1 cm³ would be predicted to release a level of ctDNA below the pre determined limit of detection of the assay. Alternatively changes in ctDNA level following treatment may be combined with this estimate to predict the tumor volume change and to determine if it is consistent with complete removal of the tumor or if it is equally constant with residual disease remaining.

The patient that provides the test sample may have cancer, may have been treated for cancer in the past (e.g., at least 2 weeks before, at least 3 months before, at least 6 months before, at least a year before), may be in complete remission and/or may have a clonal growth (e.g., a tumorous growth such as a nodule, polyp and cyst or lump) that has the potential to transform.

Likewise, the source of the cancer DNA in the sample may vary. For example, the cancer DNA may be the result of MRD, as a result of a clonal growth becoming malignant, tumor metastasis, incomplete tumor removal, or an ineffective treatment.

In some embodiments, the method may comprise providing a report indicating whether there is cancer DNA in the sample. In some embodiments, the report may contain the likelihood ratio, Bayesian posterior, score, or threshold number of variants and aliquot output described above or another number representing the same as well as a threshold to which the likelihood ratio can be compared to determine if the sample contains cancer DNA. If the report indicates there is not cancer DNA in the sample, but the likelihood ratio, Bayesian posterior, score, or threshold number of variants and aliquot output described above or another number representing the same was close to the threshold, the report may advise scheduling a follow up test in the near future to reassess if the value is now over the threshold for determining if the sample contains cancer DNA. In some embodiments, a report may additionally list approved (e.g., FDA approved) therapies for treatment of residual disease, e.g., chemotherapies or immunotherapies, etc. This information can help in diagnosing a disease (e.g., whether the patient has MRD) and/or the treatment decisions made by a physician.

In some embodiments, the report may be in an electronic form, and the method comprises forwarding the report to a remote location, e.g., to a doctor or other medical professional to help identify a suitable course of action, e.g., to diagnose a subject or to identify a suitable therapy for the subject. The report may be used along with other patients's metrics to determine whether the subject is susceptible to a therapy, for example.

In any embodiment, a report can be forwarded to a “remote location”, where “remote location,” means a location other than the location at which the sequences are analyzed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet, including email transmissions and information recorded on websites and the like. In certain embodiments, the report may be analyzed by an MD or other qualified medical professional, and a report based on the results of the analysis of the sequences may be forwarded to the patient from which the sample was obtained.

In some embodiments, a sample may be collected from a patient at a first location, e.g., in a clinical setting such as in a hospital or at a doctor's office, and the sample may be forwarded to a second location, e.g., a laboratory where it is processed and the above-described method is performed to generate a report. A “report” as described herein, is an electronic or tangible document which includes report elements that provide test results that may indicate the presence and/or quantity of cancer DNA in the sample. Once generated, the report may be forwarded to another location (which may be the same location as the first location), where it may be interpreted by a health professional (e.g., a clinician, a laboratory technician, or a physician such as an oncologist, surgeon, pathologist or virologist), as part of a clinical decision.

The patient analyzed in this method may have any type of cancer or may have previously undergone treatment for any type of cancer. For example, the patient may have or may have had melanoma, carcinoma, lymphoma, sarcoma or glioma. For example, the cancer may be melanoma, lung cancer (e.g., non-small cell lung cancer), breast cancer, head and neck cancer, bladder cancer, Merkel cell cancer, cervical cancer, hepatocellular cancer, gastric cancer, cutaneous squamous cell cancer, classic Hodgkin lymphoma, B-cell lymphoma, colorectal carcinoma, pancreatic carcinoma, gastric or breast carcinoma, among many others, including other solid tumors and blood cancers. In some embodiments the cancer is a cancer type which, on average, displays an average mutation rate of at least 0.1 mutations per megabase, or at least 0.2 mutations per megabase, or at least 0.5 mutations per megabase, or at least 1 mutation per megabase, or at least 10 mutations per megabase. Preferably, the cancer is a cancer that displays an average mutation rate of at least 0.5 mutations per megabase. Methods for calculating mutation rate are known in the art (for example Schumacher T N, Schreiber R D. Neoantigens in cancer immunotherapy. Science. 2015; 348 (6230): 69-74), hereby incorporated by reference in its entirety.

In some embodiments, the method may be used to guide treatment decisions. In some embodiments, the method may be used to determine if a patient should be treated again, e.g., with the same therapy or a second therapy. For example, if the patient has been previously been treated with a first cancer therapy and the patient has been identified as having MRD using the present method, then the patient may be treated with a second cancer therapy that is the same as or different to the first cancer therapy. For example, if the patient has previously been treated with surgery or an immune checkpoint inhibitor and the patient is identified as having MRD, then the patient may be treated with further surgery, the same or a different immune checkpoint inhibitor or another type of therapy, where immune checkpoint therapy includes administration of CTLA-4, PD1, PD-L1, TIM-3, VISTA, LAG-3, IDO or KIR checkpoint inhibitors, and the other types of therapy include, for example, (a) anthracycline therapy (e.g., by administering daunomycin, doxorubicin, or mitoxantrone), (b) alkylating agent therapy (e.g., by administering mechlorethane, cyclophosphamide, ifosfamide, melphalan, cisplatin, carboplatin, nitrosourea, dacarbazine and procarbazine or busulfan), (c) topoisomerase II inhibitor therapy (e.g., by administering etoposide or teniposide), (d) bleomycin therapy, (e) anti-metabolite therapy (e.g., by administering methotrexate, 5-fluorocil, cytarabine, 6-mercaptopurine or 6-thioguanine), (f) vinca alkyloid therapy (e.g., by administering vincrisene or vinblastine), (g) steroid therapy (e.g., by administering prednisone or dexamethasone and (h) radiation treatment, etc. Alternative therapies include targeted therapies and non-targeted chemotherapies, where targeted therapy includes treatment with erlotinib (Tarceva), afatinib (Gilotrif), gefitinib (Iressa) or osimertinib (Tagrisso) which may be administered to patients having an activating mutation in EGFR, crizotinib (Xalkori), ceritinib (Zykadia), alectinib (Alecensa) or brigatinib (Alunbrig) which may be administered to patients having an ALK fusion, crizotinib (Xalkori), entrectinib (RXDX-101), lorlatinib (PF-06463922), crizotinib (Xalkori), entrectinib (RXDX-101), lorlatinib (PF-06463922), ropotrectinib (TPX-0005), DS-6051b, ceritinib, ensartinib or cabozantinib which may be administered to patients having an ROSI fusion, or dabrafenib (Tafinlar) or trametinib (Mekinist) which may be administered to patients having an activating mutation in BRAF. Many other actionable mutations are known. If the patient is going to be switched to a non-targeted chemotherapy, the therapy may be, for example, a platinum-based doublet chemotherapy (in which the platinum-based doublet chemotherapy may comprise a platinum-based agent selected from cisplatin (CDDP), carboplatin (CBDCA), and nedaplatin (CDGP)) and one third-generation agent (selected from docetaxel (DTX), paclitaxel (PTX), vinorelbine (VNR), gemcitabine (GEM), irinotecan (CPT-11), pemetrexed (PEM), and tegafur gimeracil oteracil (S1)).

In some embodiments, the method may be used to monitor a treatment. For example, the method may comprise analyzing a sample obtained at a first timepoint using the method, and analyzing a sample obtained at a second time point by the method, and comparing the results, i.e., determining whether there is cancer DNA in the sample or determining if there is a change in the amount of cancer DNA or a range of likely amounts of cancer DNA between the first and second time points. In some embodiments, such a change may be determined using point estimates or confidence intervals and a significant decrease may indicate the therapy is effective whilst no significant decrease or an increase may indicate the therapy is not effective. The first and second timepoints may be before and after a treatment, or two or more timepoints after treatment. For example, by comparing results obtained from one timepoint to another, the method may be used to determine if the previously identified variations are no longer present, have been reduced, or have increased in the subject during the course of a treatment. The time period between the first and second timepoints may be at least one month, at least 6 months or at least one year and in some cases a patient may be tested periodically, e.g., every three months, every six months or every year for several years, e.g., 5 years or more. In another embodiment, the method may be used to evaluate the effectiveness of a treatment by monitoring patient ctDNA levels at several time intervals following treatment administration. For example, if a treatment is effective, ctDNA levels should rise shortly after administration due to cancer cell apoptosis, followed by a significant decrease as the ctDNA degrades. In such embodiments, the time period between the treatment administration and the first time point may be, e.g., at least 15 minutes, at least 30 minutes, at least 45 minutes, and at least one hour. In such embodiments, the time period between the first and second time points may be, e.g., every 15 minutes, every 30 minutes, every 45 minutes, every hour, every two hours, or ever hour for several hours, e.g. 8 hours or more.

This method may also be used to determine if a subject is disease-free, or whether a disease is recurring. As noted above, the method may be used for the analysis of minimal residual disease and recurrence detection. In these embodiments, the primer pairs used in the method may be designed to amplify sequences that contain variations that have been previously identified in a patient's cancer through either sequencing cancer material, cfDNA at an earlier time point or sequencing another suitable sample.

In some embodiments when testing for minimal residual disease or recurrence detection, the test sample of DNA from a patient would be cell-free DNA. This cell-free DNA may be taken from a patient at any point after treatment. In some embodiments this cell free DNA may be taken at a point that any remaining ctDNA from a cancer would have been cleared if the cancer were successfully treated. This time point may depend on factors such as the initial amount of ctDNA and the treatment modalities. For methods where all tumor is removed at once such as surgery time points may be after 1 week, 2 weeks, 3 weeks or 4 weeks following treatment with curative intent. Where a treatment may more gradually remove the cancer these time points may be longer such as 1 month or 2 months. As would be apparent, other DNA extracted from alternative sources could also be assessed for the presence or quantity of cancer DNA. Examples include but are not limited to: the cellular fraction of cerebrospinal fluid, the cellular and cell-free fraction of cerebrospinal fluid, stool samples, cells present within urine, biopsy or fine needle aspirate materials.

In some embodiments, the method may also be used to assess for the presence of remaining cancer cells within biopsy or fine needle aspirate materials such as from lymph nodes. As would be apparent such methods would be particularly powerful when the number of tumor cells in a biopsy sample may be at such a low level that it is not practical for histopathological analysis by a pathologist to review enough cells in the biopsy to identify the remaining cancer.

In some embodiments, the method may also be used to track a plurality of variants in parallel for example tracking predicted neoantigens-coding mutations following immunotherapy or personalized vaccine. Neoantigens are cancer-specific genetic changes, which result in an altered protein sequence, which is specific to the cancer. A personalized cancer vaccine would therefore target this altered protein sequence (or multiple, e.g. up to 20 or 30 different altered protein sequences), and teach the immune system to specifically attack the cancer cells to clear the tumour. Equally, other biological therapeutics may be useful to target noeantigens. For example, therapeutic antibodies and adoptive cell therapies (ACT, e.g. using tumour-infiltrating lymphocytes (TILs) or engineered TILs, T-cells or engineered T-cells (such as chimeric antibody receptor-engineered T-cells (CAR-T cells) or T cells with engineered T-cell receptor (TCR) fragments (TCR-Ts)) can all be generated to specifically target the cancer-specific altered protein sequences. It is important that the personalized vaccine, or other biological therapeutic results in an immune response which is specific to the cancer, and the immune response does not attack the non-cancerous tissue, thus adequate specificity of the vaccine or biological therapeutic over the native protein sequence should be ensured. Because the altered protein sequence is associated with one or more genetic changes (e.g. SNV, INDELs, fusions and other changes as mentioned herein) a personalized ctDNA assay as described herein is useful for i) initially identifying such cancer-specific genetic changes that could result in the altered protein sequence; and ii) monitoring reduction of the cancer-specific genetic change in cfDNA to indicate that the personalized vaccine, or other biological therapeutic is clearing the cancer (which may be earlier than any clinical change being observed); and iii) in the case where a personalized vaccine is designed to target multiple altered protein sequences, using the changes in ctDNA to aid the vaccine design process to confirm which of the altered protein sequences are useful in eliciting the required immune response to clear the cancer. Personalised cancer vaccines may be selected from a peptide vaccine, a DNA vaccine, an mRNA vaccine and a dendritic cell vaccine. For a review of neoantigen-based therapeutics, see for example Zhao, X., Pan, X., Wang, Y. et al. Targeting neoantigens for cancer immunotherapy. Biomark Res 9, 61 (2021) and Ott et al, An Update on Adoptive T-Cell Therapy and Neoantigen Vaccines, American Society of Clinical Oncology Educational Book 39 (May 17, 2019) e70-e78. For an example of a personalised neoantigen vaccine, see Ott P A, et al. A Phase Ib Trial of Personalized Neoantigen Therapy Plus Anti-PD-1 in Patients with Advanced Melanoma, Non-small Cell Lung Cancer, or Bladder Cancer. Cell. 2020; 183 (2): 347-62. e324, all of which are hereby incorporated by reference in their entireties.

In some embodiments, the method may be employed in a clinical trial. For example, the method may be potentially used to identify specific group of patients for clinical enrollment or evaluate the efficacy of a new drug (e.g., a neoadjuvant therapy or adjuvant therapy that may be non-specific or targeted to a patient's cancer, or any combination therapy). In some embodiments, the amount of ctDNA in a patient's bloodstream could be estimated at multiple time points thereby allowing to alter the dose of a drug administered to a patient mid-trial, for example. In some embodiments, the amount of ctDNA in a patient's bloodstream could be estimated at multiple time points during a clinical trial and used to determine if a particular therapy, level of treatment, duration of treatment or combination of treatment type and patient is working. As would be readily appreciated, many steps of the method, e.g., the sequence processing steps and the generation of a report indicating a presence of cancer DNA in a test sample of DNA may be implemented on a computer. As such, in some embodiments, the method may comprise executing an algorithm that calculates the likelihood of whether a patient has cancer DNA present in a test sample of DNA taken from a patient based on the analysis of the sequence reads, and outputting the likelihood. In some embodiments, this method may comprise inputting the sequences into a computer and executing an algorithm that can calculate the likelihood using the input measurements.

As would be apparent, the computational steps described may be computer-implemented and, as such, instructions for performing the steps may be set forth as programing that may be recorded in a suitable physical computer readable storage medium. The sequencing reads may be analyzed computationally.

The present invention also provides methods of diagnosing cancer comprising performing, on a test sample obtained from a patient, a method of detecting cancer DNA in a test sample according to a method disclosed herein.

The present invention also provides methods of treatment of cancer in a patient comprising determining the presence or absence of cancer DNA detected in a test sample from the patient according to a method disclosed herein, and administering a cancer therapy or treatment to the patient, or recommending administration of a cancer therapy or treatment to the patient. The administration or recommendation is based on the results of the cancer DNA detection method. For example, if cancer DNA is detected, then a therapy or treatment may be administered or recommended.

The present invention also provides methods of treatment of cancer in a patient, wherein the patient has been diagnosed as having or is suspected of having cancer based on the presence or absence of cancer DNA detected in a test sample from the patient as determined according to a method disclosed herein. The method comprises administering a cancer therapy or treatment to the patient based on the presence or amount of cancer DNA detected in a sample obtained from the patient. In some embodiments, the method alternatively comprises recommending a cancer therapy or treatment to the patient based on the presence or amount of cancer DNA detected in a sample obtained from the patient.

The present invention also provides methods of determining the effectiveness of a cancer treatment or therapy, comprising administering the cancer treatment or therapy to a patient, obtaining a test sample from the patient, and determining the presence, absence or amount of cancer DNA in the test sample according to a method disclosed herein. In some embodiments, the method may comprise a step of obtaining a test sample from the patient prior to the administration of the cancer treatment or therapy, and comparing the presence, absence or amount of cancer DNA in the test sample obtained before administration of the cancer therapy or treatment with the presence, absence or amount of cancer DNA in the test sample obtained after administration of the cancer therapy or treatment. A difference may be indicative of the effectiveness of the cancer therapy or treatment. For example, an increase in the amount of cancer DNA may indicate the cancer therapy or treatment is not effective. Therefore, the method may comprise administering an alternative and/or additional cancer therapy or treatment to the patient or recommending an alternative and/or additional cancer therapy or treatment for the patient. Conversely, a reduction or disappearance (that is the apparent disappearance, i.e. below the LOD of the method) of cancer DNA in the test sample may indicate the cancer therapy or treatment is effective. Therefore, the method may comprise continuing or ceasing the administration of the cancer therapy or treatment to the patient, or recommending the cancer therapy or treatment is continued or ceased. In some embodiments, the method may comprise monitoring the effect of a cancer therapy or treatment by performing the methods of cancer DNA detection using patient test sample taken from at least two time points during administration of a cancer therapy or treatment, for example test samples obtained over the course over one or more days, months or years or other time point disclosed herein.

The present invention also provides methods of detecting or monitoring minimal residual disease (MRD), comprising obtaining or having obtained a test sample from a patient that has undergone a cancer therapy or treatment, performing a method of detecting cancer DNA in the test sample according to a method disclosed herein.

The methods disclosed herein may comprise a step of obtaining a test sample from a patient. Alternatively, the test sample may have been previously obtained from the patient.

Recommendations regarding treatments or therapies may be achieved in any suitable way, for example providing a report comprising the recommendation.

Cancer therapies or treatments may be any suitable therapies. For example, the cancer treatment or therapy may be resection of a tumour. The cancer treatment or therapy may be administration of a pharmacological treatment for cancer. In some embodiments, the methods disclosed herein may be performed on a patient that has undergone surgery to remove a tumour. In some embodiments, the cancer treatment or therapy that is administered or recommended after detecting the presence or amount of cancer DNA in a test sample obtained from the patient may be a pharmacological cancer therapy or treatment.

The methods disclosed herein may be computer implemented methods, i.e., methods that are performed by or carried out on a computer.

The present invention also provides a computer-readable storage medium or media storing instructions for performing the methods disclosed herein. The computer-readable storage medium or media may be such that, when executed on a computing device, implement methods as described above. The present invention also provides a system comprising the one or more computer readable media, a memory for storing instructions to perform the method and the data units (the data units optionally comprising the one or more error probability distribution models) and a processor for executing the instructions.

### EMBODIMENTS

The disclosure provides at least the following numbered embodiments:
1. A method for detecting cancer DNA in a test sample of DNA from a patient, comprising:
(a) sequencing one or more aliquots of the test sample to produce, for each aliquot, sequence reads corresponding to two or more target regions that each have one or more sequence variations present within the patient's cancer and at least one control region;
(b) for each aliquot, for each target region:
i. determining the number of sequence reads that have the sequence variation;
ii. determining the total number of sequence reads;
iii. comparing i. and ii. to one or more error probability distribution models for the sequence variation, wherein the one or more models are obtained from DNA that does not contain the sequence variation; and
iv. optionally, eliminating variants that are above a threshold in a statistically improbable number of aliquots; and
(c) integrating the collective results of step (b) to determine if there is cancer DNA in the test sample.
2. The method of any prior embodiment, wherein step (c) comprises calculating a likelihood ratio between the likelihood of observing the estimates in (b) in samples: (i) if cancer DNA is present and (ii) if cancer DNA is not present.
3. The method of any prior embodiment, wherein integrating the collective results in step (c) comprises
   (1) determining the likelihood of observing the number of sequence reads for each aliquot and for each target region that have the one or more sequence variations if cancer DNA is present and
   (2) determining the likelihood of observing the number of sequence reads for each aliquot and for each target region if cancer DNA is not present;
   and calculating a likelihood ratio (LRᵢ) between (1) and (2).
4. The method of embodiment any prior embodiment, wherein the likelihood of observing the error probability distribution models of (b) if there is cancer DNA in the test sample is calculated based on:
   (i) the one or more error probability distribution models of step (b); and optionally
   (ii) an estimate of the cancer fraction in the test sample.
5. The method of embodiment 2, 3 or 4, wherein the likelihood of observing the estimates of (b) if there is no cancer DNA in the test sample is calculated based on:
   (i) the one or more error probability distributions of step (b); and
   (ii) an estimated rate of high signal background events.
6. The method of any one of embodiments 2 to 5, wherein the individual likelihood ratios LRi may be combined into a cumulative LR score (product of LRi equivalent to sum of log-likelihoods) across all regions and aliquots of a sample.
7. The method of any prior embodiment, wherein (c) is calculated by using a mixture model incorporating: (i) the one or more error probability distribution models step (b); and (ii) an estimated rate of high signal background events; and optionally (iii) an estimate of the cancer DNA fraction in the test sample.
8. The method of embodiments 2 to 7, wherein step (c) further comprises comparing the likelihood ratio to a threshold, wherein an output that is at or above the threshold indicates that the test sample contains cancer DNA.
9. The method of embodiment 8, further comprising identifying the test sample as having cancer DNA if the result is at or above the threshold.
10. The method of any one of embodiments 8 to 9, wherein the threshold is determined by using control samples from a healthy donor who is assumed to not have cancer as test samples and selecting a threshold above the signal identified in the control samples.
11. The method of embodiment 10, wherein the threshold is selected such that the false positive rate as determined using the control samples is estimated to be 1% or below, 0.1% or below or 0.01% or below.
12. The method of any one of embodiments 10 to 11, wherein the number of control samples is at least 10, or at least 100, or at least 1000, or at least 10,000 samples.
13. The method of any one of embodiments 10 to 12, wherein the control samples are from the same patient.
14. The method of any one of embodiments 10 to 12, wherein the control samples are from different patients.
15. The method of any one of embodiments 2 to 14, wherein
   a. a likelihood ratio is calculated for one or more alternative variants in each control sample;
   b. the likelihood ratios for the one or more alternative variants are combined to give an overall likelihood ratio for a subject assumed not to have cancer; and
   c. the threshold is set above the overall likelihood ratio for a subject assumed not to have cancer patient.
16. The method of embodiment 15, wherein the number of alternative variants is approximately the same as the number of target regions.
17. The method of any one of embodiments 8 to 16, wherein the threshold is calculated in advance from a pool of subjects assumed not to have cancer.
18. The method of any prior embodiment, wherein the error probability distribution model comprises a confidence score, wherein the confidence score comprises a threshold which is obtained from DNA that does not contain the sequence variation.
19. The method of embodiment 18, wherein step (c) comprises calling a target region as positive for the sequence variation when the confidence score threshold for the sequence variation is exceeded.
20. The method of embodiment 19, wherein the test sample is called positive for containing cancer DNA when at least two target regions are called positive.
21. The method of any one of any prior embodiment, wherein the error probability distribution model comprises at least a first error distribution model and a second error distribution for each sequence variation.
22. The method of embodiment 21, wherein the first error distribution model comprises an estimated background error rate.
23. The method of embodiment 21 or embodiment 22, wherein the second error distribution model is a high signal background event.
24. The method of any prior embodiment, further comprising administering a cancer therapy or treatment to the patient.
25. The method of any prior embodiment wherein the level of cancer DNA released from a tumor prior to treatment is correlated with tumor volume prior to treatment as assessed by imaging in order to give an estimate of the amount of ctDNA released by a set volume of tumor and thus a standardised measure of tumor ctDNA release.
26. The method of any prior embodiment, wherein the patient has previously undergone a first cancer treatment or therapy.
27. The method of embodiment 26, further comprising administering a second cancer therapy or treatment that is different to the first cancer treatment or therapy to the patient.
28. The method of any prior embodiment, wherein the method further comprises determining the amount of cancer DNA or a range of likely amounts of cancer DNA in the test sample based on the collective results of step (c).
29. The method of embodiment 28, wherein determining the amount of cancer DNA or a range of likely amounts of cancer DNA in the test sample based on the collective results of step (c) comprises estimating a mean or median cancer DNA variant allele fraction.
30. The method of embodiment 28 or 29, wherein determining the amount of cancer DNA or a range of likely amounts of cancer DNA in the test sample based on the collective results of step (c) comprises maximum likelihood analysis.
31. The method of embodiment 28, 29 or 30, wherein determining the amount of cancer DNA or a range of likely amounts of cancer DNA in the test sample based on the collective results of step (c) comprises Bayesian posterior analysis.
32. The method of any one of embodiments 28 to 31, wherein determining the amount of cancer DNA or a range of likely amounts of cancer DNA in the test sample based on the collective results of step (c) comprises counting the number of estimated mutant molecules for each variant and each aliquot.
33. The method of any one of embodiments 28 to 32, wherein determining the amount of cancer DNA or a range of likely amounts of cancer DNA in the test sample based on the collective results of step (c) is done by counting the number of variant positive target regions in each aliquot and comparing this against the total number of target regions multiplied by aliquots and quantifying the mean number of variant containing target sequences per target region per aliquot by applying a Poisson correction to the fraction of the positive results.
34. The method of any prior embodiment, wherein the method is performed on samples that are obtained from the patient at at least a first time point and a second time point, wherein the first time point is prior to a treatment and the second time point is after the treatment, and the method comprises determining if there is a change in the amount of cancer DNA or a range of likely amounts of cancer DNA between the first and second time points.
35. The method of embodiment 34, wherein, the method is performed on further samples obtained at additional time points, wherein additional samples are taken after the second time point on a monthly, bimonthly, quarterly, or annual schedule.
36. The method of embodiment 34 or 35, wherein a change is determined using point estimates or confidence intervals, and wherein a significant decrease indicates the therapy is effective and no significant change or an increase indicates the therapy is not effective.
37. The method of any one of embodiments 34 to 36, wherein a change of at least 20%, at least 30%, at least 50%, at least 70% or at least 90% is considered significant.
38. The method of any one of embodiments 34 to 37, wherein a change is considered significant if the change is greater than 50% and confidence intervals when quantifying cancer DNA for the first and second time point do not overlap.
39. The method of any one of embodiments 24 to 38, further comprising generating a report indicating whether the cancer therapy or treatment is effective or not.
40. The method of any prior embodiment, further comprising generating a report indicating the presence or absence of cancer DNA in the test sample.
41. The method of any prior embodiment, wherein the sequence variations that are identified in a statistically improbable number of the aliquots are excluded from the results of step (b) prior to step (c).
42. The method of any prior embodiment, wherein the sequence variations that are identified in a statistically improbable number of the aliquots are determined based on the estimated cancer DNA fraction and/or the number of DNA molecules added to each aliquot, optionally the number of times each variant is represented in an individual cancer cell as determined through copy number analysis.
43. The method of any prior embodiment, wherein step (a) comprises sequencing at least 2, 3, 4, 5, 6, 7, 8, 9, 10, 11 or 12 aliquots.
44. The method of any prior embodiment, wherein step (a) comprises sequencing at least four aliquots.
45. The method of any one of embodiment 1 to 42, wherein step (a) comprises sequencing one aliquot.
46. The method of any prior embodiment, wherein step (b) (iv) comprises using the copy number of each of the one or more sequence variations to estimate the threshold for the statistically improbable number of aliquots.
47. The method of any prior embodiment, wherein step (a) also comprises sequencing positive and or negative control samples which may include at least one of: cancer DNA from an aspirate, biopsy or surgery sample coming from the same patient, buffy coat DNA, buccal swab DNA, whole blood DNA, and adjacent non-cancerous DNA.
48. The method of embodiment 47, wherein the sequencing of the control samples is performed at the same time as the test sample.
49. The method of embodiment 47, wherein the sequencing of the control samples is performed before or after sequencing the test sample.
50. The method of any one of embodiments 47 to 49, wherein the negative control sample is buffy coat DNA, which is optionally sequenced at the same time as the test sample.
51. The method of any one of embodiments 47 to 50 wherein the negative control sample is a blood product from a healthy donor assumed to not have cancer which is optionally sequenced before the test sample.
52. The method of any one of embodiments 47 to 51, wherein the positive control sample is cancer DNA taken from a biopsy from the same patient as the test sample, which is optionally sequenced before the test sample.
53. The method of any one of embodiments 47 to 52, wherein each of the control samples are sequenced as a single sample, as opposed to aliquots.
54. The method of any one of embodiments 47 to 53, wherein variants that are not detected in the cancer DNA control samples are excluded from the determination of whether there is cancer DNA in the test sample in step (c).
55. The method of any one of embodiments 47 to 54, wherein variants detected in the buffy coat, buccal swab, adjacent non-cancerous, whole blood control samples, or other negative control sample are excluded from the determination of whether there is cancer DNA in the test sample in step (c).
56. The method of any prior embodiment, wherein the two or more target regions is at least 2, at least 4, at least 10, at least 20, at least 50, at least 100, at least 500, at least 1,000 or at least 5,000 target regions.
57. The method of any prior embodiment, wherein the two or more target regions is from about 2 to about 200 target regions.
58. The method of any prior embodiment, wherein the two or more target regions is from about 6 to about 100 target regions.
59. The method of any prior embodiment, wherein the sequence variations of step (a) are independently selected from the list consisting of single nucleotide variants, indels, transpositions, and rearrangements.
60. The method of any prior embodiment, wherein the sequence variations of step (a) are single nucleotide variants and/or indels.
61. The method of any prior embodiment, wherein the sequence variations are pre-identified sequence variations.
62. The method of any prior embodiment, wherein the sequence variations are epigenetic variants.
63. The method of any prior embodiment, wherein a plurality of candidate sequence variations are identified by sequencing: (i) DNA isolated from a tissue biopsy that comprises tumor cells, (ii) DNA isolated from a tumor tissue obtained at surgery that comprises tumor cells or (iii) sequencing cell-free DNA or (iv) DNA isolated from circulating tumor cells.
64. The method of embodiment 63, wherein the candidate sequence variations are identified by sequencing a whole genome, a whole exome, or a region of a genome selected due to commonly containing cancer mutations.
65. The method of embodiment 63 or 64, wherein the candidate sequence variations are identified by sequencing the entire exome of cancer DNA from a tissue biopsy or other surgical sample.
66. The method of any one of embodiments 63 to 65, wherein the candidate sequence variations are first identified, scored, ranked, and selected based on one or more of: allele fraction; clonality; mappability; estimated background error rate; estimated high signal background error rate; distance from another selected variant; predictive ability to sequence; presence within a region of copy number gain or amplification; and proximity of any germ line variants which may be used for enriching the mutant allele.
67. The method of any prior embodiment, wherein a target region is selected when it comprises 2 or more sequence variations or candidate sequence variations that are sufficiently close to one another to be positioned on a single sequence read, optionally wherein the sequence read is up to approximately 160 bp in length.
68. The method of any prior embodiment, wherein a target region is selected when it comprises 2 or more sequence variations or candidate sequence variations that are present less than 10 bp apart, less than 50 bp apart or less than 100 bp apart.
69. The method of any prior embodiment, wherein the method further comprises sequencing at least some of the target regions in the DNA of white blood cells from the patient, comparing candidate sequence variations to the sequence variations identified using the white blood cell DNA and optionally eliminating any candidate sequence variations identified in both the white blood cells and the test sample.
70. The method of any one of embodiments 64 to 69, wherein the whole exome is divided into windows and the windows are scored, ranked and selected based on one or more of: allele fraction; clonality; mappability; estimated background error rate; estimated high signal background error rate; distance from another selected variant; predictive ability to sequence; presence within a region of copy number gain or amplification; and proximity of any germ line variants which may be used for enriching the mutant allele.
71. The method of embodiment 70, wherein the windows are at least 10, 50, 100, 1000 or 10000 base pairs in length.
72. The method of embodiment 70 or 71, wherein the windows are overlapping, optionally by at least 5, 10, 25, or 50 base pairs.
73. The method of any one of embodiments 70 to 72, wherein the windows are from about 20 base pairs to about 100 base pairs and overlap by about half the length of the full window.
74. The method of any one of embodiments 70 to 73, wherein each variant is scored, and a score for the window is generated by combining the scores for all variants within the window and optionally combining this with a score or scores for region specific features which may include mappability, predictive ability to sequence and presence within a region of copy number gain or amplification.
75. The method of any prior embodiment, wherein the patient has or had cancer or has a clonal growth that is not yet cancer but has the potential to transform.
76. The method of any prior embodiment, wherein the patient has undergone or is undergoing treatment or therapy for the cancer.
77. The method of any prior embodiment, wherein the DNA is cell-free DNA.
78. The method of any prior embodiment, wherein the DNA is circulating DNA.
79. The method of any prior embodiment, wherein the DNA is circulating cfDNA.
80. The method of any prior embodiment, wherein the DNA is ctDNA.
81. The method of any prior embodiment, wherein the DNA (optionally, the cell-free DNA) is isolated from blood plasma, blood serum, cerebrospinal fluid, urine, saliva, stool, amniotic fluid, aqueous humour, bile, breast milk, cerumen, chyle, exudates, gastric juice, lymph, mucus, pericardial fluid, peritoneal fluid, pleural fluid, pus, sebum, serous fluid, semen, sputum, synovial fluid, sweat, tears, vomit, or whole blood.
82. The method of any prior embodiment, wherein the cancer DNA is cell-free DNA isolated from blood plasma.
83. The method of any prior embodiment, wherein the fraction of cancer DNA in the test sample of DNA is equal to or less than 1%.
84. The method of any prior embodiment, wherein the fraction of cancer DNA in the test sample of DNA is at least about 0.0001%, optionally to about 1%.
85. The method of any prior embodiment, wherein the test sample comprises less than 25,000 genome equivalents of DNA.
86. The method of any prior embodiment, wherein the number of aliquots and the maximum number of molecules per aliquot is adjusted based on the total number of input molecules and the estimated background error rate such that the number of input molecules in a single aliquot is low enough that if a single variant molecule were present it would produce a signal statistically significantly different to background.
87. The method of any prior embodiment, wherein for each aliquot of each sequence variation, the read depth of step (a) is at least 10,000.
88. The method of any prior embodiment, wherein for each aliquot of each sequence variation, the read depth of step (a) is from about 10,000 to about 500,000.
89. The method of any prior embodiment, wherein for each aliquot of each sequence variation, the read depth of step (a) is from about 10,000 to about 200,000.
90. The method of any prior embodiment, further comprising, quantifying the amount of DNA in the test sample prior to step (a).
91. The method of any prior embodiment, wherein the test sample of DNA is enriched for the target regions and control regions prior to step (a).
92. The method of embodiment 91, wherein the test sample of DNA is enriched by PCR or by hybridization to a nucleic acid probe.
93. The method of any prior embodiment, wherein the cancer is a solid tumour.
94. The method of any one of embodiments 1 to 92, wherein the cancer is a haematological cancer.
95. The method of any prior embodiment, wherein the sequencing step comprises appending molecular barcodes to the DNA in the or each aliquot.
96. The method of any prior embodiment, wherein the or each aliquot comprises from about 100 to about 10000 amplifiable copies of the genome of the patient.
97. The method of embodiment 96, wherein the or each aliquot comprises from about 500 to about 5000 amplifiable copies of the genome of the patient.
98. The method of any prior embodiment, wherein the total amount of DNA present in the or each aliquot is used to select the one or more error probability distribution models for the sequence variation.
99. The method of any prior embodiment, comprising quantifying the total amount of cancer DNA present in the or each aliquot.
100. The method of embodiment 99, wherein the total amount of cancer DNA present in the or each aliquot is quantified using a mean or median variant allele fraction across the variants and aliquots, optionally where the mean or median variant allele fraction is a corrected mean or median variant allele fraction.
101. The method of any prior embodiment further comprising, for each aliquot and target region, estimating the number of molecules of DNA that have the sequence variation in the test sample or the probability that there is at least one molecule that has the sequence variation.
102. The method of embodiment 101, wherein the estimation is determined using (i) and (ii) of step (b), and an estimated background error rate for the sequence variation.
103. The method of any one of embodiments 66 to 102, wherein the background error rate may be expressed by at least one error probability distribution.
104. The method of embodiment 103, wherein there are at least two error probability distribution models.
105. The method of embodiment 104, wherein the at least two error probability distribution models are of the same type.
106. The method of embodiment 104, wherein the at least two error probability distribution models are of different types.
107. The method of embodiment 106, comprising an error probability distribution model for a background error rate and an error probability distribution model for an estimated rate of high signal background events.
108. The method of any prior embodiment, wherein the one or more error probability distribution models and/or the background error rate is estimated by reference to data from the at least one control region.
109. The method of any prior embodiment, wherein the one or more error probability distribution models and/or the estimated background error rate may be estimated by analysis of sequence reads corresponding to the at least one control region produced in step (a).
110. The method of any prior embodiment, wherein the one or more error probability distribution models and/or estimated background error rate is a probability distribution over the number of variant molecules present.
111. The method of any prior embodiment, wherein the probabilities for sequence variations that are identified in a statistically improbable number of the aliquots based on the estimated cancer DNA fraction are excluded, prior to calculating likelihood of there being cancer DNA in the sample, or prior to determining if sufficient target regions, variants and/or aliquots are above a threshold to indicate cancer DNA is present.
112. The method of any prior embodiment, wherein the one or more error probability distribution models further comprises a high signal background event error rate, and wherein each target region and aliquot contributes a result to step (c) based on the high signal background event error rate.
113. The method of embodiment 112, wherein a result comprises an indication of whether the sequence variation is present in the test sample.
114. The method of any one of embodiments 112-113, wherein a result is one if the sequence variation is present and zero if the sequence variation is not present.
115. The method of any one of embodiments 112-114, wherein comparing i. and ii. to one or more error probability distribution models for the sequence variation comprises determining a score for a sequence variation based on the high signal background event error rate.
116. The method of any one of embodiments 112-115, wherein determining a score further comprises weighting a result based on the high signal background event error rate.
117. The method of any one of embodiments 112-116, wherein weighting a result based on the high signal background event error rate comprises weighting the result by 1 if there are no high signal background events.
118. The method of any one of embodiments 112-117, wherein weighting a result based on the high signal background event error rate comprises weighting the result by less than 1 if there are one or more high signal background events.
119. The method of any one of embodiments 112-118, wherein the sequence variations are separated into at least three groups based on the high signal background event error rate, wherein the three groups comprise: 1) no high signal background events; 2) a low rate of high signal background events; and 3) a high rate of high signal background events.
120. The method of embodiment 119, wherein the weight for group 1 is 1.0, the weight for group 2 is 0.75, and the weight for group 3 is 0.50.
121. The method of any prior embodiment, wherein integrating the collective results of step (b) comprises summing the result or score for each aliquot and for each target region.
122. The method of any one of embodiments 112-121, wherein integrating the collective results of step (c) further comprises determining there is cancer DNA in the sample if the collective result is at least two.
123. The method of any one of embodiments 112-122, wherein integrating the collective results of step (c) further comprises determining there is cancer DNA in the sample if the collective result is at least three.
124. A method for detecting cancer DNA in a test sample of DNA from a patient, comprising:
   a. providing sequence reads derived from one or more aliquots of the test sample, wherein, for each aliquot, the sequence reads comprises sequences corresponding to two or more target regions that each have one or more sequence variations present within the patient's cancer and at least one control region;
   b. for each aliquot, for each target region:
      i. determining or having determined the number of sequence reads that have the sequence variation;
      ii. determining or having determined the total number of sequence reads;
      iii. comparing or having compared i. and ii. to one or more error probability distribution models for the sequence variation, wherein the one or more models are obtained from DNA that does not contain the sequence variation; and
      iv. optionally, eliminating or having eliminated variants that are above a threshold in a statistically improbable number of aliquots; and
   c. integrating or having integrated the collective results of step (b) to determine if there is cancer DNA in the test sample;
   d. providing a report summarizing the results of step (c).
125. The method of embodiment 124, wherein the method is as defined in any one of embodiments 1 to 123.
126. The method of embodiment 124 or 125, wherein the method is a computer implemented method.
127. A method for detecting cancer DNA in a test sample of DNA from a patient, comprising:
   a. providing sequence reads derived from one or more aliquots of the test sample wherein, for each aliquot, the sequence reads comprise sequences corresponding to two or more target regions that each have one or more sequence variations present within the patient's cancer and at least one control region;
   b. for each aliquot, for each target region:
      i. determining or having determined the number of sequence reads that have the sequence variation;
      ii. determining or having determined the total number of sequence reads;
      iii. comparing or having compared i. and ii. to one or more error probability distribution models for the sequence variation, wherein the one or more models are obtained from DNA that does not contain the sequence variation; and
      iv. optionally, eliminating or having eliminated variants that are above a threshold in a statistically improbable number of aliquots; and
   c. integrating the collective results of step (b) to determine if there is cancer DNA in the test sample.
128. The method of embodiment 127, wherein the method is as defined in any one of embodiments 1 to 123.
129. The method of embodiment 127 or 128, wherein the method is a computer implemented method.
130. A computer-readable storage medium or media storing instructions for performing the method of claim 129
131. The computer-readable storage medium or media of embodiment 130, wherein the instructions further comprise instructions for providing a report summarizing the results of step (c)
132. A computer system comprising the computer-readable storage medium of embodiment 130 or 131.
133. A computer system configured to perform the method of any one of embodiments 126 or 129.
134. A method for detecting cancer DNA from a test sample collected from a cancer patient, the method comprising:
   (a) sequencing the test sample to produce sequence reads corresponding to two or more target regions, wherein each target region comprises a sequence variation associated with the patient's cancer;
   (b) for each target region, identifying sequence reads containing a sequence variation associated with the patient's cancer and calling the target region as positive for cancer DNA if the identified sequence reads indicate the sequence variation is present in the sample; and
   (c) determining whether there is cancer DNA in the sample based on the number of positive target regions.
135. The method of embodiment 134, wherein the two or more target regions are selected by sequencing cancerous and non-cancerous samples collected from the patient; and comparing the sequenced cancerous and non-cancerous samples to identify the sequence variations associated with the patient's cancer.
136. The method of embodiment 135, wherein comparing the sequenced cancerous and non-cancerous samples further comprises confirming that a plurality of germline variants are present in both samples.
137. The method of embodiment 136, wherein the plurality of germline variants comprises between 10 and 100 single nucleotide polymorphisms (SNPs).
138. The method of any one of embodiments 134-137, wherein comparing the cancerous and non-cancerous samples to identify the one or more sequence variations comprises inferring the clonality of a sequence variation.
139. The method of any one of embodiments 134-138, where at least 50% of the identified sequence variations are clonal.
140. The method of any one of embodiments 134-139, wherein selecting the two or more target regions further comprises ranking the sequence variations associated with the patient's cancer.
141. The method of embodiment 140, wherein the one or more sequence variations are ranked based on at least one of: variation allele frequency (VAF) in cancer DNA; sequence adjacent to the sequence variation; and an efficiency rate of PCR amplification.
142. The method of any one of embodiments 134-141, wherein the ratio of the number of positive target regions to the total number of target regions required to call the test sample as positive for cancer DNA is at least one-eighth (⅛).
143. The method of any one of embodiments 134-142, wherein the number of positive target regions required to call the test sample as positive for cancer DNA is at least two target regions.
144. The method of any one of embodiments 134-143, wherein the total number of target regions is sixteen.
145. The method of any one of embodiments 134-144, wherein calling a target region as positive for cancer DNA comprises comparing the number of sequence reads containing the sequence variation to an estimated background error rate.
146. The method of embodiment 145, wherein a target region is called as positive for cancer DNA if the quantity of sequence reads containing the sequence variation exceeds an expected quantity of sequence reads containing the sequence variation based upon an estimated background error rate.
147. The method of any one of embodiments 145-146, wherein the estimated background error rate is calculated based on at least one of: an efficiency rate of PCR amplification; a probability that each molecule is replicated in a PCR cycle; an error rate per cycle for a particular mutation type; and an initial number of molecules.
148. The method of any one of embodiments 145-147, wherein the estimated background rate is selected for each sequence variation based on a class comprising the sequence adjacent to the sequence variation.
149. The method of any one of embodiments 145-148, wherein the estimated background rate is determined using from one or more control samples.
150. The method of embodiment 149, wherein the one or more control samples comprises at least 10, at least 20, at least 50, at least 100, or at least 1000 control samples.
151. The method of any one of embodiments 145-148, wherein comparing the number of sequence reads containing the one or more variations to an estimated background rate comprises calculating a confidence score.
152. The method of embodiment 151, wherein the confidence score comprises the likelihood of a variation to be present in the test sample at a given variant allele fraction (L(θ)).
153. The method of embodiment 152, further comprising selecting a value for θ that maximizes the likelihood (\hat{θ}_{MLE}).
154. The method of any one of embodiments 151-153, wherein the confidence score further comprises the likelihood of a sequence variation to not be present in the test sample (L(θ)), the confidence score comprising:

$$\frac{L ( {\hat{θ}}_{MLE} )}{L ( 0 ) + L ( {\hat{θ}}_{MLE} )} .$$

155. The method of any one of embodiments 151-154, wherein a target region is called as positive for cancer DNA if the confidence score for the sequence variation associated with the patient's cancer exceeds a predetermined threshold.
156. The method of any one of embodiments 134-155, further comprising quantifying the amount of cancer DNA in the sample.
157. The method of embodiment 156, wherein quantifying the amount of cancer DNA in the test sample comprises calculating the mean variant allele fraction (mean VAF).
158. The method of embodiment 156 or 157, wherein quantifying the amount of cancer DNA in the test sample comprises calculating the mean number of tumor molecules per volume.
159. The method of embodiment 158, wherein the mean number of tumor molecules per volume (MTM) is calculated by: 303×cfDNA

$$( \frac{ng}{mL} ) \times Mean V A F .$$

160. The method of any one of embodiments 134-155, wherein the test sample is prepared by a multiplexed PCR reaction to amplify each variant using target-specific primers and a barcoding PCR reaction to add test sample barcodes.
161. The method of embodiment 160, wherein the barcoding PCR reaction uses primers targeting the tails of primer sequences in the multiplexed PCR reaction.
162. The method of any one of embodiments 160-161, further comprising enriching the test sample for low molecular weight fragments.
163. The method of any one of embodiments 134-162, wherein the test sample is sequenced to a depth of approximately 100,000×.
164. The method of any one of embodiments 134-163, wherein the cancer DNA is circulating tumor DNA (ctDNA).
165. A method for detecting cancer DNA from a test sample collected from a cancer patient, the method comprising:
   (a) sequencing or having sequenced the test sample to produce sequence reads corresponding to two or more target regions, wherein each target region comprises a sequence variation associated with the patient's cancer;
   (b) for each target region, identifying or having identified sequence reads containing a sequence variation associated with the patient's cancer and calling the target region as positive for cancer DNA if the identified sequence reads indicate the sequence variation is present in the sample;
   (c) determining or having determined whether there is cancer DNA in the sample based on the number of positive target regions;
   (d) providing a report summarizing the results of step (c).
166. The method of embodiment 165, wherein the method is as defined in any one of embodiments 134 to 164.
167. The method of embodiment 165 or 166, wherein the method is a computer implemented method.
168. A method for detecting cancer DNA from a test sample collected from a cancer patient, the method comprising:
   (a) sequencing or having sequenced the test sample to produce sequence reads corresponding to two or more target regions, wherein each target region comprises a sequence variation associated with the patient's cancer;
   (b) for each target region, identifying or having identified sequence reads containing a sequence variation associated with the patient's cancer and calling the target region as positive for cancer DNA if the identified sequence reads indicate the sequence variation is present in the sample;
   (c) determining whether there is cancer DNA in the sample based on the number of positive target regions.
169. The method of embodiment 168, wherein the method is as defined in any one of embodiments 134 to 164.
170. The method of embodiment 168 or 169, wherein the method is a computer implemented method.
171. A computer-readable storage medium or media storing instructions for performing the method of claim 170
172. The computer-readable storage medium or media of embodiment 171, wherein the instructions further comprise instructions for providing a report summarizing the results of step (c)
173. A computer system comprising the computer-readable storage medium of embodiment 171 or 172.
174. A computer system configured to perform the method of any one of embodiments 167 or 170.
175. A method of diagnosing cancer in a patient, comprising performing the method of any prior embodiment on a test sample obtained from the patient.
176. A method of treating cancer in a patient, comprising determining the presence or absence of cancer DNA in a test sample according to the method of any one of embodiments 1 to 170, and administering a cancer therapy or treatment to the patient, or recommending administration of a cancer therapy or treatment to the patient.
177. A method of treatment of cancer in a patient, comprising administering a cancer therapy or treatment to a patient, or recommending a cancer therapy or treatment to the patient, wherein the patient has been diagnosed as having cancer or suspected of having cancer according to the method of embodiment 176.
178. A method of determining the effectiveness of a cancer treatment or therapy, comprising administering the cancer treatment or therapy to a patient, obtaining a test sample from the patient, and determining the presence, absence or amount of cancer DNA in the test sample according to the method of any one of embodiments 1 to 170.
179. The method of embodiment 178, comprising obtaining a test sample from the patient prior to administration of the cancer therapy or treatment, determining the presence, absence or amount of cancer DNA in the test sample obtained before administration of the cancer therapy or treatment according to the method of any one of embodiments 1 to 170, and comparing the presence, absence or amount of cancer DNA in the sample obtained before administration of the cancer therapy or treatment with the presence, absence or amount of cancer DNA in the sample obtained after administration of the cancer therapy or treatment.
180. A method of monitoring the effect of a cancer therapy or treatment comprising administering the cancer therapy or treatment to a patient and performing the method of cancer DNA detection according to any one of embodiments 1 to 170 using test samples obtained from the patient at two or more time points during or after the administration of the cancer therapy or treatment.
181. A method of detecting or monitoring minimal residual disease (MRD), comprising obtaining or having obtained a test sample from a patient that has undergone a cancer therapy or treatment, and performing a method of detecting cancer DNA in the test sample according to the method of any one of embodiments 1 to 170.
182. The method according to any one of embodiments 134 to 164, wherein the method is further defined according to any one of embodiments 2 to 129.

### EXAMPLES

The following examples are put forth so as to provide those of ordinary skill in the art with a complete disclosure and description of how to make and use the present invention, and are not intended to limit the scope of what the inventors regard as their invention.

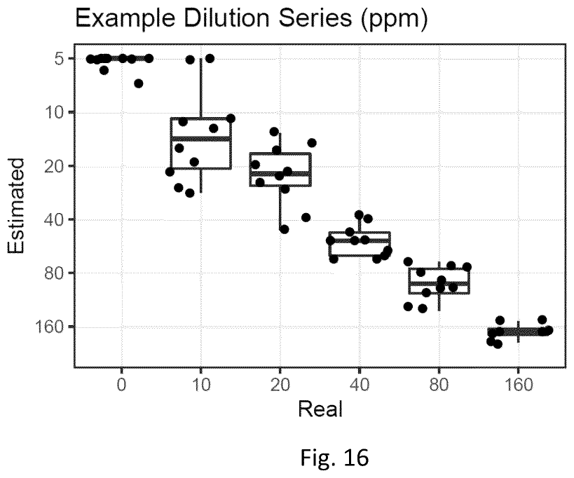

FIG. 15 shows why calling a sample as containing cancer DNA can be challenging, particularly for samples that have a low tumor fraction. As shown in the top panel, samples that have a high tumor fraction (TF), cancer DNA can be readily called because several positive signals are obtained in multiple aliquots. This eliminates most false positives. As shown in the bottom panel, samples that have a low tumor fraction are more difficult to call since the data may be accounted for by the background error rates. For example, if each positive variant has a 80% probability of corresponding to an actual sequence variation, the evidence shown for the low tumor fraction sample in FIG. 15 is insufficient to call the sample as containing cancer DNA. However, if the evidence is aggregated across multiple variants and aliquots there may be sufficient evidence to call a sample as containing cancer DNA.

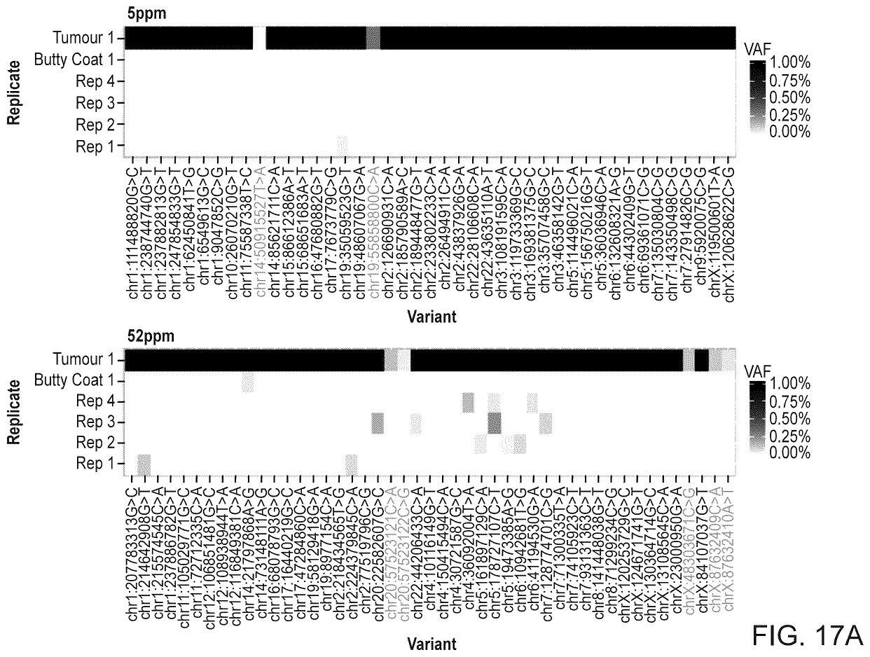

FIG. 11 shows an embodiment of how evidence can be combined across multiple variants. For dilute samples (<<0.1% tumor fraction), the fraction of mutant reads for individual variants in each sample is not expected to approximate the overall tumor fraction because of dropout effects. For example, many aliquots will contain zero variant molecules. Instead, the effect of taking n/input reads per aliquot as a discrete distribution is modeled. In this example the tumor fraction is not measured directly. Rather, it is marginalized over all possible inputs, which provides an accurate estimate of the tumor fraction of the sample. Specifically, instead of guessing the number of variant molecules, the probabilities of all possible values are calculated based on: (i) the number of sequencing reads that have the sequence variation; (ii) the total number of sequencing reads; (iii) the number of molecules input into each aliquot; and (iv) the estimated background error rate for the sequence variation, and the value with the highest probability is identified. This avoids making assumptions. In FIG. 15, the variants are shown as present or absent for each aliquot. However, these are in fact probabilities which take into account many factors such as tumor fraction and per-base noise estimates. A ground truth line (FIG. 16) can be constructed. FIG. 14 shows that particularly noisy variations, i.e., variations that are identified in a statistically improbable number of the aliquots can be excluded from the analysis.

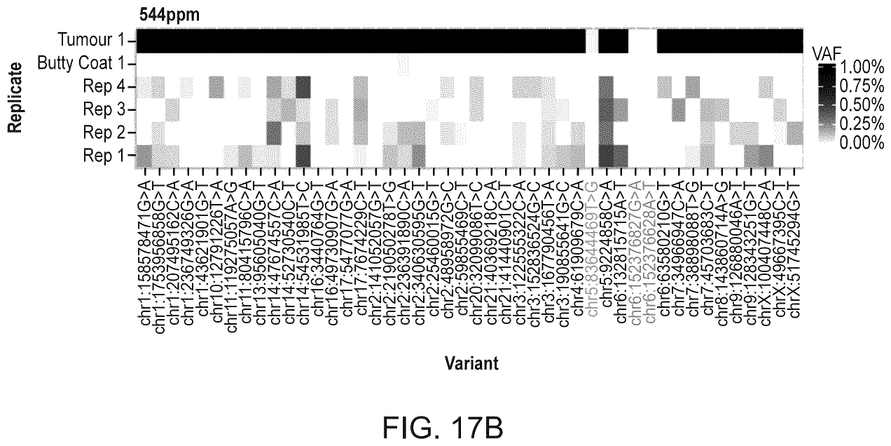

FIGS. 17A-B show the results of an experiment in which over 40 sequence variations in four aliquots of each of three different samples containing varying levels of circulating tumor DNA (ctDNA), were analyzed using the present method. The 52 ppm and 544 ppm samples are identified as having ctDNA, which illustrates the advantage of combining evidence across multiple aliquots and variants. In this figure, the color intensity correlates with the VAF (variant allele fraction), with the brightest color representing >=1%. Some variant names are greyed out in order to indicate their absence in the original tumor sample.

### Example 1

In order to build an optimal assay for detecting residual disease, the cancer type of interest, in this instance, breast cancer was first selected. The mutational rate of the cancer was reviewed and identified to be over 0.5 mutation per Mb in approximately 90% of patients with the average patient having over 1 mutation per Mb (Martincorena and Campbell, Science 2015 349: 1483-9). In a pilot study of 22 early breast cancer patients it was identified that ctDNA is detected at a median of 0.06% VAF and down to 0.0007% VAF.

Studies diluting 3 cancer cell lines into non-cancerous DNA were performed using a personalized assay tracking 48 variants demonstrating that cancer DNA can be detected consistently at 0.001% VAF when analyzing 48 variants in combination but that the level of sensitivity halves each time the number of variants halve.

Based on the mutational rate of breast cancer, the observation that ctDNA is detected ~50% of the time at below 0.06% and is detectable all the way down to 0.0007% VAF in the pilot study, a target of at least 90% of breast cancer samples having a limit of detection of at least 0.001% VAF was set. With a mutation rate of 0.5 mutations per Mb, a 96 Mb region of the genome was required for sequencing in breast cancer.

The main advantage of this approach include reproducibly achieving the levels of sensitivity needed for the cancer type of interest as in at least 90% of patients ≥48 variants are identified. Another advantage is that when a sample with a lower mutation rate is targeted, sequencing costs can be reduced.

### Example 2

In order to design the optimal MRD assay, the system is designed to interrogate as many high quality variants as is possible. In order to do this a tumor biopsy is first obtained, it is macro-dissected targeting 50% tumor content, exome capture is performed then the sample is sequenced using an Illumina® sequencer. All potential variants are identified using standard Illumina® pipelines then given a combined score based on 1) the likelihood of being real, 2) the likelihood of being somatic, 3) the background error rate for the variant, 4) the high signal background error rate, 5) the probability of being clonal, 6) the level of amplification or copy number gain of the variant. The genome is divided into 50 bp windows and these windows overlap by 25 bp. Each window is given a combined score that includes 1) the scores of all variants present within the window, 2) a score for the ability to uniquely align the region (where penalty is given for regions that cant be uniquely aligned and the penalty is higher, the greater the number of mis alignments), 3) a score for the ability to amplify and sequence the region (where penalty is given to features know to challenge sequencing including repeats). The regions are then sorted by score and the top 100 are selected for designing PCR primers to. Where 2 regions that overlap are in the top 100 list, the region with the highest score is maintained and the region with the weaker score is discarded. The 101^{st} region is then added to the list and so on. A multiplex PCR is designed for the top 48 variants. Insilico PCR is performed using all primer pairs. When primer combinations are identified producing ≥2 non specific regions, the primer for the lowest scoring region which is causing this non specific product is discarded and alternative primers designed. If non overcome the non specific PCR problem, the region is discarded and the next region is added to the primer design.

One challenge with this tumor informed method of detecting cancer DNA in a test sample is the number of regions that can be robustly and cost effectively targeted, This strategy of ranking regions could maximize the number of variants that are successfully interrogated in the test DNA sample. When the variants are in cis (next to each other on the same chromosome) they can be read together and this increases the ability to separate signal from noise. When the variants are in trans, but still readable with the same primer pairs (or other targeting reagents like baits) the amount of information from the single targeted region should be doubled. The approach should also limit the number of reads wasted on non-specific products.

### Example 3

In order to detect cancer DNA in test samples with high sensitivity, it is advantageous targeting multiple variants. For some cancer types it is sufficient to target just one type of variant. Sometimes though it is better to target multiple types of variants. In this example, it is identified that for certain breast cancer patients, a large number of structural variants are present, whilst in other patients there are more SNVs and indels. A large panel is designed to sequence breast cancer tumor DNA assessing for SNVs, indels and rearrangements. The optimal variant containing regions are identified. Primers are designed to target these regions. Where the regions contains 1 or more SNVs/indels, the primers are designed to flank all the SNVs and indels. Where the “region” is identified to contain a rearrangement, two different parts of the same chromosome or two different chromosomes will have been brought together. The rearrangement sequence is used for primer design and one primer is 3′ of the rearrangement and one is 5′. In instances where an SNV, indel or other variant (e.g. DBSs) is in cis with the rearrangement, the primers are designed to flank both the rearrangement and other variant(s) using the rearranged sequence obtained from the tumor. An advantage of this approach is the ability to consistently obtain a large number of variants for assessment of cancer DNA in a test sample.

### Example 4

In order to determine both the background error rate and the rate of high signal background events, 50 different panels, each with 48 amplicons are designed. Each of the panels is designed against the exome of a patient that has either lung, CRC or breast cancer. Each amplicon in the panel is on average ~100 bp long and within this there is on average ~60 bp of sequence that is readable from the test DNA (i.e. non primer sequence). Blood is obtained from 200 healthy donors assumed to not have cancer. Each donors blood is drawn into a Streck® cell free DNA blood collection tube. The blood is spun to plasma, cell free DNA is extracted then the DNA is quantified by digital PCR. Each panel is tested with the cfDNA from 4 donors. A multiplex PCR with multiple aliquots (3) is setup using the panel and cfDNA. This PCR is barcoded. The barcoded products from patients is pooled together. These are run on an Illumina® NovaSeq® sequencer. The variants types to be assessed for are agreed as SNVs and indels. These variants are split into the following classes: Type of SNV (e.g. C>A, T>A or G>A), type and size of indel (e.g. 1 bp, 2 bp, 3 bp del etc.). The results from the donors are split into 3 groups (low DNA input, medium DNA input and high DNA input) based on digital PCR quantification of the cfDNA. Excluding primer sequences, a buffer of 3 bp and all location wherein a potential germline variant has been reported in gnomAD, for the remaining bases at each location the total number of reads, the number of each non reference base and the count of each different type/size of indel are obtained. For each change (e.g. C>A) a beta distribution is fitted to the data. Both the mean and CV are obtained. Using a cumulative distribution function (CDF) for the particular base change a threshold of 0.9999 is used to determine an allele fraction cutoff at which the sample must be to be considered positive. This is the background error rate. To determine the rate of high signal background events, for each change (e.g. C>A), all instances of the change in the test panels are assessed and the rate of detecting a signal above the CDF determined allele fraction threshold is calculated.

### Example 5

A panel is designed for the tumor of a breast cancer patient by obtaining a biopsy sample and sequencing 96 Mb of the tumor's genome, then selecting primers to amplify 48 regions wherein in total, the 48 regions include 50 variants (SNVs and indels) believed to be somatic and specific to the tumor. The patient specific primers are multiplexed and a multiplex PCR is setup using the cancer DNA. The PCR products are barcoded then sequenced on an Illumina® sequencer. The variants not detected in the cancer DNA are bioinformatically filtered. The same panel is applied to the buffy coat DNA from the patient. A library is generated and sequenced. All variants identified at over 40% VAF are flagged as germline and filtered. All variants identified over the allele fraction cutoff as determined by the variant type and background error rate but below 40% are flagged as likely clonal hematopoiesis of indeterminate potential and filtered. If greater than 12 variants remain following the filtering, the panel is applied to the cfDNA extracted from the patient (if fewer remain, a panel redesign is attempted). CfDNA is split into 3 aliquots and a multiplex PCR performed using the patient specific primers on all 3 aliquots. The PCR products are barcoded, bead cleanup is performed then samples are pooled and sequenced. At the completion of sequencing, the reads are demultiplexed, trimmed, filtered based on quality and aligned to the reference genome. At each target region, for all variants in each target region, the number of wild type reads and the total number of reads are counted.

### Example 6

Following the completion of sequencing of 3 aliquots of cfDNA from a breast cancer patient the total number of mutant and total reads for all aliquots of all variants excluding those filtered variants are obtained. The Variant allele fraction (mutant/total reads) is determined then this variant allele fraction is compared to the threshold generated using the background error rate. All aliquots for all variants are assessed to determine if they are positive or negative (above the threshold). The tumor fraction is estimated by first correcting all VAFs using the background error rate then averaging across all aliquots of all variants. The number of DNA molecules added to each library preparation is compared with the average VAF to determine how likely it is we would expect at least one mutant molecule in each aliquot of each variant. Each variant is then assessed to determine if there are more positive aliquots than would be expected by chance and those that are determined to have an improbable number of positive aliquots (P<0.05) are filtered. A score of 1 is then given to any variants who have no high signal background events (e.g. typically indels). For the remaining variants, they are separated into those with a high rate of “high signal background events” (the top 50%) and those with a low rate of “high signal background events” (all those that are in the bottom 50% excluding those that have no “high signal background events”. All variants with a low rate contribute a score of 0.75 and those with a high rate contribute a score of 0.5. If the test DNA sample is determined to have a total score of equal or greater than 2 and if at least 2 aliquots have a score of 0.5 or greater the test sample is deemed to have cancer DNA. There are a number of advantages of such an approach. In some approached one could simply determine if enough variants are above a threshold (e.g. 2 variants above a threshold). This is limited as some variants commonly produce high signal background events whilst others never do. This approach therefore enables confident calling with high specificity when just 2 variants are detected when these variants never produce high signal background events. When the variants identified are more prone to high signal background events the scoring approach is therefore more cautious and between 3 and 4 variants are needed in order to make a call enabling the assay to maintain high specificity. By requiring a score in more than one aliquot the assay prevents false positives due to contamination of a single aliquot whilst filtering out variants that are either present in buffy coat or present in more aliquots than is likely based on the estimated tumor fractions, common sources of false positives including CHIP and error prone bases are eradicated.

### Example 6

Following the completion of sequencing of 3 aliquots of cfDNA from a breast cancer patient the total number of mutant and total reads for all aliquots of all variants excluding those filtered variants are obtained. The Variant allele fraction (mutant/total reads) is determined then this variant allele fraction is compared to the threshold generated using the background error rate. All aliquots for all variants are assessed to determine if they are positive or negative (above the threshold). The tumor fraction is estimated by first correcting all VAFs using the background error rate then averaging across all aliquots of all variants. The number of DNA molecules added to each library preparation is compared with the average VAF to determine how likely it is we would expect at least one mutant molecule in each aliquot of each variant. Each variant is then assessed to determine if there are more positive aliquots than would be expected by chance and those that are determined to have an improbable number of positive aliquots (P<0.05) are filtered. A calling threshold for the number of variants is then determined by obtaining the estimated rate of high signal background events for all remaining unfiltered variants then calculating a distribution of the likely number of high signal background events across all remaining aliquots and variants. A threshold number of positive variants is then obtained wherein there is less than 0.01% change of obtaining the number of positive events purely through high signal background events. The sample is then called positive if the total number of positive variants (variants above VAF threshold) is above this threshold number of positive variants and if at least 2 aliquots have a positive variant. There are a number of advantages of such an approach. In some approached one could simply determine if enough variants are above a threshold (e.g. 2 variants above a threshold). This is limited as some variants commonly produce high signal background events whilst others never do. This approach therefore enables confident calling by estimating how commonly high signal background events would be present and with what distribution. A personalized threshold is then set depending on how noisy the variants are and how many variants there are. This enables very high sensitivity but also balances this with specificity (for example when a large number of variants with common high signal background events are tested the threshold is higher than when a small number of variants that rarely have high signal background events is tested). By requiring a positive in more than one aliquot the assay prevents false positives due to contamination of a single aliquot whilst filtering out variants that are either present in buffy coat or present in more aliquots than is likely based on the estimated tumor fractions, common sources of false positives including CHIP and error prone bases are eradicated.

### Example 7

FFPE tumor material is obtained. The tissue is sectioned and total RNA is extracted from 10 slides. Ribosomal RNA depletion, reverse transcription and sequencing library preparation is performed. The sequencing library is barcoded then multiplexed with other libraries from patients. Sequencing on an Illumina® NovaSeq® platform is performed. The reads are demultiplexed, aligned then the variants called. The variants include SNVs, indels and gene fusions. These variants are then mapped from their RNA transcripts to the correct genomic DNA coordinates for primer design.

### Example 8

Paired samples of tumor tissue (FFPE) and whole blood are obtained from a set of cancer patients. The whole blood samples are collected in K2-EDTA 10 mL tubes (Beckton Dickinson) and plasma is isolated within 2 hours of blood collection by double centrifugation (buffy coat is collected after the first centrifugation). DNA is extracted from the FFPE samples using the QIAamp DNA FFPE tissue kit (Qiagen), from the plasma samples using the QIAamp circulating nucleic acid kit (Qiagen), and from the buffy coat sample using the QIAamp DNA blood kit (Qiagen). Tissue and buffy coat DNA are quantified by the Qubit dsDNA BR Assay Kit (ThermoFisher) and plasma cfDNA using the Quant-IT high sensitivity dsDNA assay kit (Invitrogen).

A median of 500 ng of DNA from each tumor and buffy coat sample are subjected to whole-exome sequencing (WES) (Agilent, 200 ng DNA protocol), and the resulting sequence reads are quality checked using FastQC, aligned to the human reference genome (hg19) using the Burrows-Wheeler Alignment tool, and further quality checked using Picard and MultiQC. Additionally, a set of 45 SNVs are genotyped from each patient in both tumor and plasma to ensure sample concordance.

Patient-specific somatic variants are identified by comparing tumor (cancerous) and buffy coat (non-cancerous) DNA WES profiles for all patients. Clonality of variants is inferred based on the estimated proportion of cancer cells harboring the variant, though this can be limited due to samples with low tumor cell fractions. Somatic variants (including SNVs and INDELs) are ranked based on observed VAFs in cancer DNA and local sequence context, such as the uniqueness (and thus mappability) of the sequence surrounding the variant, as well as the expected efficiency of PCR primers for amplifying that site. Once ranked, the top 16 variants are selected to create a patient-specific variant panel and a pair of PCR primers are designed to amplify each variant.

Plasma cfDNA is eluted into 50 μL buffer. The extraction is optimized for low molecular weight fragments to minimize potential contamination from white blood cells and/or to maximize the number of short molecules recovered. cfDNA libraries are prepared using up to 66 ng of cfDNA (approximately 20,000 genomes) and subjected to blunting, A-tailing, and adapter ligation, followed by amplification and purification using Ampure XP beads (Agencourt/Beckman Coulter). While this step only amplifies dsDNA present in plasma, it leads to a more robust assay in that the resulting sample can be sequenced alone (without enriching for specific targets), or can be used to test multiple pairs of PCR primers to compare efficiency rates and degree of target amplification.

Each library is then subjected to a multiplexed PCR reaction to amplify each variant using target-specific primers, followed by a barcoding PCR reaction (targeting the tails of the target-specific primers) to add sample barcodes. Barcoded samples were subsequently pooled, purified, and quantified with Qubit dsDNA HS assay kit (Life Technologies).

The resulting libraries are sequenced at an average depth per amplicon of 100,000× per variant using an Illumina® platform. Sequence reads are aligned to the human reference genome (hg19) using BWA-mem v0.7.10 (Li & Durbin 2019). For each somatic variant in the panel, the number of variant reads (n) and number of total reads (N) are counted and compared to a target-specific error model including a background error model and an error propagation model. The background error model is built by estimating PCR efficiency, the probability of each molecule being replicated in a PCR cycle, the error rate, the per-cycle error rate for a particular mutation type (e.g. wild-type A to mutant allele G), and a starting number of molecules. The error propagation model characterizes the distribution of error molecules and estimates the mean and variance of the total number of molecules and total number of error molecules after n PCR cycles.

To call somatic variants, PCR efficiency and the per-cycle error rate are estimated from a set of non-cancerous control samples, followed by estimating the starting number of molecules and PCR efficiency in the cfDNA sample. The mean and variance for the total number of molecules, background error molecules, and real mutation molecules are then estimated using the error propagation model for a range of potential VAF values for the variant. Finally, this mean and variance are used to compute the likelihood L(θ) for each potential VAF and the VAF value that maximizes this likelihood (designated \hat{θ}_{MLE}) was selected. A confidence score for each variant is then calculated as follows:

$$\frac{L ( {\hat{θ}}_{MLE} )}{L ( 0 ) + L ( {\hat{θ}}_{MLE} )} .$$

Any variants exceeding a predetermined threshold (validated to ensure high specificity while maintaining high sensitivity) are called positive. Once all variants are considered, the cfDNA sample is called as positive for cancer DNA if two or more variants out of the sixteen total are positive. This ratio (at least one-eighth) of positive variants to the total number works well given the expected level of ctDNA typically present in cfDNA samples and represents a good balance of specificity and sensitivity, the probability of seeing two false positive variants in a set of sixteen is exceedingly low.

Additionally the mean number of tumor molecules (MTM) present per mL is quantified based on 1) the amount of cfDNA (ng), an estimate of the number of haploid genomes (1 ng=303 haploid genomes), and an estimate of the mean VAF per plasma volume (mL), as follows:

$$M T M = cfDNA ( ng ) \times \frac{Haploid Genome Equivalent ( h G E )}{0.0 \; 033 ng per h G E} \times \frac{Mean V A F}{Plasma volume ( mL )} ,$$

which is equivalent to:

$$303 \times cfDNA ( \frac{ng}{mL} ) \times Mean V A F .$$

Mean VAF is estimated based on the VAF of all positive variants in the panel.

### Example 9

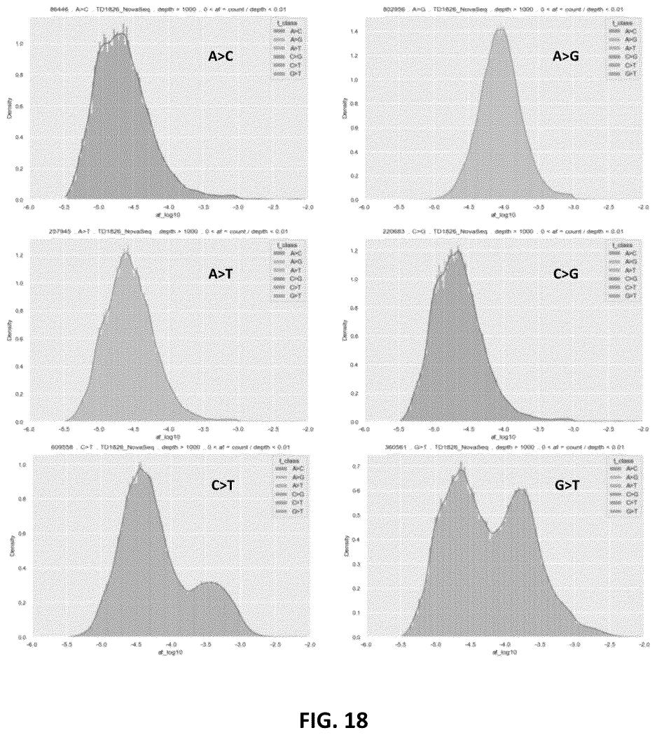

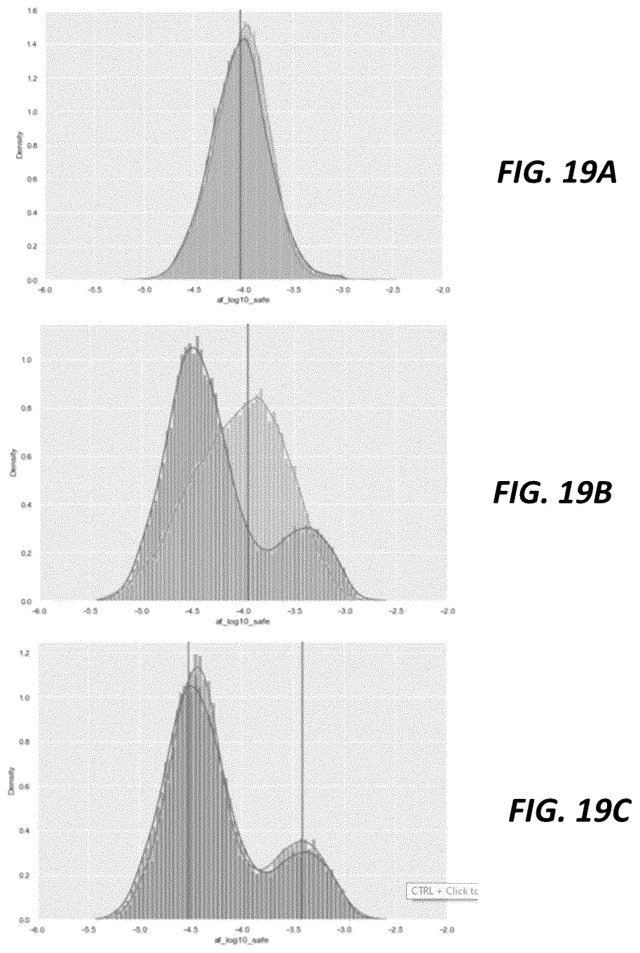

Several control samples (non-cancerous DNA) were sequenced on the same sequencing run as the 3 aliquots of cfDNA from a breast cancer patient as previously described. After sequencing, the sequence reads for the control samples were mapped to the human genome reference sequence and all base substitutions were identified and separated into six classes: A>C, A>G, A>T, C>G, C>T, and G>T substitutions. A histogram was then generated for log₁₀ (count/depth) per substitution class, as shown in FIG. 18. The histograms show the level of background error for each substitution class. As shown, most substitution classes contain only a single maximum, indicating a single source of error (or alternately, multiple sources of error that may be accounted for by a single error distribution). In contrast, the C>G and G>T substitution classes contain a distinct second source of error, as evidenced by two peaks. Beta-binomial distributions were then generated from the control data for each substitution class to account for the observed error. A single beta-binomial distribution can accommodate substitution classes having single peaks (FIG. 19A), but will not accurately accommodate those with two peaks (FIG. 19B). To accommodate the second source of error observed in the C>G and C>T substitutions, additional beta-binomial distributions were generated with shape parameters to accommodate the observed second peaks (e.g., FIG. 19C). The sequencing data from the cfDNA from the breast cancer patient were then processed according to an embodiment of the disclosure using these generated error probability distributions.

1. A method comprising:
(a) obtaining a biological sample of a subject;
(b) extracting one or more nucleic acid molecules from the biological sample of the subject, wherein the one or more nucleic acid molecules comprise deoxyribonucleic acid (DNA) molecules;
(c) dividing the DNA molecules or derivatives thereof into at least two sample aliquots;
(d) determining a concentration of the DNA molecules or derivatives thereof in an aliquot of the at least two sample aliquots, such that a single DNA molecule of the DNA molecules or derivatives thereof generates a signal distinguishable from a signal threshold when the single DNA molecule comprises a genetic variant, wherein the signal threshold corresponds to a background error rate for the at least two sample aliquots;
(e) modifying a quantity of molecules in an aliquot of the at least two sample aliquots based at least in part on the concentration determined in (d); and
(f) performing an amplification reaction on at least a subset of the DNA molecules or derivatives thereof in the at least two sample aliquots, thereby generating a sequencing library comprising at least two enriched sample aliquots.

2. The method of claim 1, wherein the biological sample is obtained or derived from a tumor tissue of the subject.

3. The method of claim 1, wherein the biological sample comprises a blood sample.

4. The method of claim 3, wherein the blood sample is whole blood.

5. The method of claim 3, wherein the blood sample comprises a blood plasma sample or a blood serum sample.

6. The method of claim 1, wherein the biological sample comprises a buccal swab sample, a bone marrow sample, or a tumor tissue sample.

7. (canceled)

8. The method of claim 1, wherein the biological sample comprises a set of biological samples.

9. The method of claim 8, wherein a first biological sample of the set of biological samples comprises a tumor tissue sample of the subject.

10. The method of claim 9, wherein a second biological sample of the set of biological samples comprises a normal tissue sample of the subject.

11. The method of claim 10, further comprising obtaining the second biological sample from an area adjacent to an area of the first biological sample.

12. The method of claim 1, wherein the DNA molecules comprise buffy coat DNA molecules.

13. The method of claim 1, wherein the DNA molecules comprise cell-free DNA (cfDNA) molecules.

14. (canceled)

15. The method of claim 1, further comprising subjecting the sequencing library to nucleic acid sequencing, thereby generating one or more sequence reads for the at least subset of the at least two sample aliquots.

16. (canceled)

17. (canceled)

18. The method of claim 1, wherein the subject is a human subject.

19. The method of claim 18, wherein the human subject is a human subject having cancer.

20. The method of claim 1, wherein the at least two sample aliquots comprise between two and ten sample aliquots.

21. The method of claim 1, wherein the at least two sample aliquots comprise more than ten sample aliquots.

22. The method of claim 1, wherein the quantity of molecules comprises a maximum number of DNA molecules or derivatives thereof in an aliquot of the at least two sample aliquots.

23. The method of claim 15, wherein the nucleic acid sequencing comprises next-generation sequencing (NGS) or exome sequencing.

24. The method of claim 23, further comprising processing the sequence reads to detect the genetic variant.

25. The method of claim 24, wherein the genetic variant comprises a single nucleotide variant (SNV).

26. The method of claim 25, further comprising determining a genomic region of the sequence reads having the genetic variant.

27. The method of claim 26, further comprising determining a number of sequence reads in a subset of the sequence reads having the determined genomic region and the genetic variant.

28. The method of claim 27, further comprising determining a total number of the sequence reads.

29. The method of claim 28, further comprising determining one or more error probability distributions for the determined number of the subset of sequence reads as compared to the total number of the sequence reads.

30. The method of claim 29, further comprising determining a first error probability distribution of the one or more error probability distributions comprising the determined genomic region and the genetic variant.

31. The method of claim 30, further comprising determining a second error probability distribution of the one or more error probability distributions for a distinct subset of the sequence reads not having the genetic variant.

32. The method of claim 1, wherein the amplification reaction comprises a multiplex polymerase chain reaction (PCR).
